(19) United States
(12) Patent Application Publication
(10) Pub. No.: US 2025/0372199 A1
(43) Pub. Date: Dec. 4, 2025

(54) ALLELIC IMBALANCE OF CHROMATIN ACCESSIBILITY IN CANCER IDENTIFIES CAUSAL RISK VARIANTS AND THEIR MECHANISMS

(71) Applicant: DANA-FARBER CANCER INSTITUTE, INC., Boston, MA (US)

(72) Inventors: Alexander Gusev, Boston, MA (US); Dennis Grishin, Boston, MA (US)

(21) Appl. No.: 18/873,188

(22) PCT Filed: Jun. 9, 2023
(86) PCT No.: PCT/US2023/024957

§ 371 Date: Dec. 9, 2024

Related U.S. Application Data
(60) Provisional application No. 63/351,201, filed on Jun. 10, 2022.

(51) Int. Cl. G16B 20/20; G16B 30/00; G16B 40/20; G16H 10/40; G16H 50/30; G16H 50/70

(52) U.S. Cl. CPC G16B 20/20; G16B 30/00; G16B 40/20; G16H 10/40; G16H 50/30; G16H 50/70

(57) ABSTRACT

The disclosure provides a method for determining whether a subject is at risk of developing or will develop cancer. The biomarker may be a single nucleotide polymorphism (SNP). An SNP may be referred to by an rsID number, which is a unique number used to identify a specific SNP. Information about SNPs with rsID numbers are maintained by the National Library of Medicine, which provides the SNP's position in the genome, the alleles present (the reference nucleotide in a so-called wild-type condition and the altered nucleotide), the frequency at which the altered nucleotide has previously been detected, and its type.

### CROSS-REFERENCE TO RELATED APPLICATIONS

This application claims the benefit of priority under 35 U.S.C. § 119(e) to U.S. Provisional Application No: 63/351,201, filed Jun. 10, 2022, which is incorporated herein by reference in its entirety.

### GOVERNMENT LICENSE RIGHTS

This invention was made with government support under grant number R01HG006399, R01CA244596, R01MH115676, and R01CA227237 awarded by the National Institutes of Health. The government has certain rights in the invention.

### BACKGROUND OF THE DISCLOSURE

Genome-Wide Association Studies (GWAS) are observational studies of a genome-wide set of genetic variants in multiple subjects with the goal of associating a genetic variant with a trait or disease. GWAS produces a wealth of information relating, in part, to complex genetic traits. While GWAS has identified many germline cancer risk variants, GWAS itself does not elucidate the underlining mechanisms by which these variants operate.

GWAS of cancer have identified hundreds of risk loci (Zhang, H. et al., Nat. Genet. 52, 572-581 (2020); Michailidou, K. et al., Nature 551, 92-94 (2017); Conti, D. V. et al., Nat. Genet. 53, 65-75 (2021); Mckay, J. D. et al., Nat. Genet. 49, 1126-1132 (2017); Sud, A., Kinnersley, B. & Houlston, R. S. Nat. Rev. Cancer 17, 692-704 (2017)), but the underlying mechanisms remain largely unknown, with only a handful of functionally validated risk variants (Michailidou, K. et al., Nature 551, 92-94 (2017); Fachal, L. et al., Nat. Genet. 52, 56-73 (2020)). Therefore, there is an urgent need for new strategies to identify and validate risk variants to a cancer and cancer mechanism.

### SUMMARY OF THE DISCLOSURE

In one aspect, the disclosure provides a method for determining whether a subject is at risk of developing or will develop cancer. The biomarker may be a single nucleotide polymorphism (SNP). An SNP may be referred to by an rsID number, which is a unique number used to identify a specific SNP. Information about SNPs with rsID numbers are maintained by the National Library of Medicine, which provides the SNP's position in the genome, the alleles present (the reference nucleotide in a so-called wild-type condition and the altered nucleotide), the frequency at which the altered nucleotide has previously been detected, and its type.

In some embodiments, the method may entail obtaining a test sample from a subject having or at risk of having a cancer, determining the presence of a biomarker in the test sample, where the biomarker is selected from the group consisting of the biomarkers set forth in Table 8A-Table 8G, determining that the subject is at risk of developing cancer based on the presence of the biomarker. In some embodiments, the method may also entail administering to the subject a therapeutically effective amount of one or more cancer therapies. In some embodiments, a cancer therapy of the one or more cancer therapies is surgery. In some embodiments, a cancer therapy of the one or more cancer therapies is hormone therapy. In some embodiments, a cancer therapy of the one or more cancer therapies is immunotherapy. In some embodiments, a cancer therapy of the one or more cancer therapies is chemotherapy. In some embodiments, a cancer therapy of the one or more cancer therapies is radiotherapy. In some embodiments, the value of the biomarker is of a sequence, concentration, expression level, peak intensity, or chromatin accessibility.

In some embodiments, the biomarker is a genetic variant identified by an rsID, or wherein the biomarker comprises a genetic variant identified by a peak ID. In some embodiments, the biomarker is within a gene region, that gene region having a gene and a promoter. In some embodiments, the gene in the gene region is in the catalogue of somatic mutations in cancer (COSMIC). In some embodiments, the biomarker is within a gene. In other embodiments, the biomarker is within a promoter region. In some embodiments, the biomarker is within a transcription factor (TF) binding motif or disrupts a TF binding motif. In some of these embodiments, the TF motif is bound by a member of the Runt, Ets, APETALA 2 (AP2), basic leucine zipper (bZIP), zinc-finger (Zf), or E2 Factor (E2F) families. In some embodiments, the TF binding motif is bound by KLF14, PIT1, RBPJ, RUNX, RUNX1, SP2, SP5, ZNF263, or ZNF467. In some embodiments, the biomarker is rs2981578, or rs2992756.

In some embodiments, the biomarker is selected from the group consisting of the biomarkers set forth in Table 8A, and the cancer is breast cancer. In some embodiments, the biomarker is a genetic variant that disrupts a TF binding motif selected from the group consisting of AP-1, AP-2α, AP-2γ, ATF3, BACH1, BACH2, BATF, CRX, CSCL, E2F1, E2F3, E2F4, E2F6, EHF, ELF1, ELF4, ETS1, ETV4, FAFB, FLI1, FOS, FOSL2, FOXO1, FRA1, FRA2, HAND2, JUN, JUNB, KLF4, KLF14, MAFA, MAFK, NANOG, NF-E2, NFE2L2, NRF2, PIT1, PITX1, PU.1, RBPJ, RUNKX1, SIX2, SPDEF, SP2, SP5, ZNF 263, ZNF467, and ZNF675. In some embodiments, the biomarker is rs2981578, rs11599804, rs10787473, rs12258200, rs1316014, rs1314913, rs62090606, rs249473, rs2494734, rs1462985, rs2992756, or rs3767812. In some embodiments, the surgery is sentinel lymph node biopsy, breast-conserving surgery, total mastectomy, or modified radical mastectomy. In some embodiments, the hormone surgery is ovarian ablation, tamoxifen, luteinizing hormone-releasing hormone (LHRH) agonists, aromatase inhibitors, or combinations thereof. In some embodiments, a cancer therapy is a targeted therapy.

In some embodiments, the biomarker is selected from the group consisting of the biomarkers set forth in Table 8B, and the cancer is prostate cancer. In some embodiments, the surgery is radical prostatectomy, pelvic lymphadenectomy, or transurethral resection of the prostate. In some embodiments, the hormone therapy is abiraterone acetate, estrogens, luteinizing hormone-releasing hormone agonists, antiandrogens, orchiectomy, or combinations thereof. In some embodiments, a cancer therapy is abiraterone, bicalutamide, leuprolide, apalutamide, degarelix, flutamide, cabazitaxel, lutetium Lu 177 vipivotide tetraxetan, Olaparib, mitoxantrone, nilutamide, darolutamide, relugolix, sipuleucel-T, radium 223 Dichloride, rucaparib camsylate, docetaxel, enzalutamide, goserelin, or combinations thereof.

In some embodiments, the biomarker is selected from the group consisting of the biomarkers set forth in Table 8C, and the cancer is colorectal cancer. In some embodiments, the surgery is local excision, anastomosis, or colostomy. In some embodiments, a cancer therapy is a monoclonal antibody, angiogenesis inhibitor, protein kinase inhibitor, or combinations thereof. In some embodiments, a cancer therapy is bevacizumab-maly, bevacizumab, irinotecan, Ramucirumab, oxaliplatin, cetuximab, 5-FU, ipilimumab, pembrolizumab, leucovorin, trifluridine and tipiracil hydrochloride, nivolumab, regorafenib, panitumumab, capecitabine, ziv-aflibercept, or a combination thereof.

In some embodiments, the biomarker is selected from the group consisting of the biomarkers set forth in Table 8D, and the cancer is renal cancer. In some embodiments, the surgery is partial nephrectomy, simple nephrectomy, or radical nephrectomy. In some embodiments, a cancer therapy is bevacizumab-maly, bevacizumab, irinotecan, ramucirumab, oxaliplatin, cetuximab, 5-FU, ipilimumab, pembrolizumab, leucovorin, trifluridine and tipiracil hydrochloride, nivolumab, regorafenib, panitumumab, capecitabine, ziv-aflibercept, or a combination thereof. In some embodiments, the biomarker is selected from the group consisting of the biomarkers set forth in Table 8E, and the cancer is glioma. In some embodiments, a cancer therapy is everolimus, bevacizumab-maly, bevacizumab, carmustine, naxitamab-gqgk, carmustine implant, lomustine, temozolomide, and belzutifan, or combinations thereof.

In some embodiments, the biomarker is selected from the group consisting of the biomarkers set forth in Table 8F, and the cancer is lung cancer. In some embodiments, the surgery is wedge resection, lobectomy, pneumonectomy, or sleeve resection. In some embodiments, a cancer therapy is paclitaxel albumin-stabilized nanoparticle formulation, everolimus, alectinib, pemetrexed disodium, brigatinib, bevacizumab, amivantamab-vmjw, ramucirumab, doxorubicin hydrochloride, mobocertinib succinate, pralsetinib, afatinib dimaleate, gemcitabine, durvalumab, gefitinib, pembrolizumab, cemiplimab-rwlc, lorlatinib, sotorasib, trametinib dimethyl sulfoxide, nivolumab, necitumumab, selpercatinib, entrectinib, capmatinib, dabrafenib mesylate, osimertinib mesylate, erlotinib, docetaxel, atezolizumab, tepotinib hydrochloride, methotrexate, dacomitinib, vinorelbine tartrate, crizotinib, ipilimumab, ceritinib, or combinations thereof. In some embodiments, the biomarker is selected from the group consisting of the biomarkers set forth in Table 8G, and the cancer is melanoma. In some embodiments, a cancer therapy is encorafenib, cobimetinib fumarate, dacarbazine, talimogene haherparepvec, recombinant interferon alfa-2b, pembrolizumab, tebentafusp-tebn, trametinib dimethyl sulfoxide, binimetinib, nivolumab, nivolumab and relatlimab-rmbw, peginterferon alfa-2b, aldesleukin, dabrafenib mesylate, ipilimumab, vemurafenib, or combinations thereof.

In another aspect, the disclosure provides a method for treating cancer in a subject. In some embodiments, the method may entail obtaining a test sample from a subject having or at risk of having a cancer, determining the presence of a biomarker in the test sample, where the biomarker is selected from the group consisting of the biomarkers set forth in Table 8A-Table 8G, determining that the subject is at risk of developing cancer based on the presence of the biomarker, and administering to the subject a therapeutically effective amount of one or more cancer therapies.

In another aspect, the present disclosure may provide a method of detecting a biomarker in a subject. In some embodiments, the method may entail obtaining a test sample from the subject and detecting whether a biomarker is present in the test sample by sequencing DNA from the sample and comparing the biomarker with the DNA sequence from the test sample, where the biomarker comprises a sequence of DNA that is selected from the group consisting of the biomarkers set forth in Table 8A-Table 8G.

Without intending to be bound by theory, it is hypothesized that the inventive bioinformatics tools and methods of use thereof may identify genomic regions that exhibit imbalance of chromatin accessibility, enabling the discover genomic regions that play a role in the regulation of gene expression. Such imbalanced regions are more strongly enriched for cancer risk heritability than any other functional annotation (e.g., eQTLs). The inventive bioinformatics tools enable the connection of imbalanced (regulatory, non-coding) genomic regions to cancer risk, and by combining imbalance analysis and RWAS, candidate causal cancer risk variants may be identified. Furthermore, deep learning-based models enable the prediction of germline genetic variants or somatic mutations that result in allelic imbalance and affect gene expression, thusly identifying regulatory non-coding germline genetic variants and somatic mutations without having to conduct new experiments.

The stratAS platform may be adapted to perform allelic imbalance analysis to cancer samples (e.g., ATAC-Seq samples) to characterize allelic imbalance in cancer types. stratAS is a method that leverages individual read counts across samples to significantly increase the statistical power of even moderately sized studies (van de Geijn, B. et al., Nat. Methods 12, 1061-1063 (2015); Kumasaka, N. et al., Nat. Genet. 48, 206-213 (2016)). Using the inventive approaches described herein, thousands of non-coding germline genetic variants, termed allelic-specific accessibility QTLs (as-aQTLs), may be analyzed to identify new biomarkers for cancer. These as-aQTLs may be more enriched for cancer risk heritability than any other molecular feature tested, across GWAS data from cancer types. The as-aQTLs described herein affect cancer risk and progression. Motif analyses may confirm causation of as-aQTLs with genetic variants that altered the binding of TFs and affected gene expression. This extensive regulatory activity may be linked to cancer risk through prediction via a Regulome-Wide Association Study (RWAS) and integration of allelic imbalance and TF motif discovery. RWAS enables the identification of candidate causal cancer risk variants and their cis-regulatory mechanisms. The germline variants discovered herein affect the binding of cancer-linked transcription factors that remain active in cancer. Such interactions may implicate risk mechanisms that are difficult to observe in steady-state normal tissues, as well as cancer-specific epigenetic dysregulation associated with disease progression.

In one embodiment, the identification of 7,262 germline as-aQTLs was possible using the inventive methods described herein from 406 cancer ATAC-seq samples across 23 cancer types. The working examples show that cancer as-aQTLs have stronger enrichment for cancer risk heritability (up to 145-fold) than any other functional annotation across seven cancer GWAS. The majority of cancer as-aQTLs directly altered TF motifs and exhibited differential TF binding and gene expression in functional screens. To connect as-aQTLs to putative risk mechanisms, RWAS were performed, which identified genetically associated accessible peaks at >70% of known breast and prostate loci and discovered novel risk loci in all examined cancer types. Methods integrating as-aQTL discovery, motif analysis, and RWAS identified candidate causal regulatory elements and their likely upstream regulators. This disclosure establishes cancer as-aQTLs and RWAS analysis as powerful tools to study the genetic architecture of cancer risk.

### BRIEF DESCRIPTION OF THE DRAWINGS

FIG. 1A-FIG. 1E are a series of schematics, bar, and line plots showing that stratAS identifies as-aQTLs in cancer samples. FIG. 1A is a schematic showing that, for a given heterozygous germline SNP i in an individual, an imbalance likelihood L can be calculated by modeling the reads overlapping the site as coming from a beta-binomial distribution: L (REF, ALT, σᵢ), where counts of REF and ALT reads are used to calculate allelic ratios, and ρᵢ is a locally defined, per-individual overdispersion parameter. It is incorporated into the allelic imbalance test, to account for copy number variations (CNVs) and other causes of sequencing read over-dispersion. FIG. 1B is a schematic showing that significantly imbalanced regions are identified if they exhibit consistent allelic effects across samples. Random somatic events such as allelic deletions or duplications are not expected to influence imbalance analysis results except in extreme cases. FIG. 1C is a bar plot showing that the number of as-aQTLs and d-as-aQTLs, and the intersection of those two sets discovered in 23 cancer types (abbreviations defined in Table 1). The number of samples for each cancer type is shown in brackets. d-as-QTLs are not a perfect subset of as-aQTLs because a peak can be a d-as-aQTL but not an as-aQTL. This occurs when there is no imbalance in tested cancer type, while there is imbalance in other cancer types. FIG. 1D and FIG. 1E are two dot plots showing that a representative breast cancer-specific as-aQTL/d-as-aQTL at the variant rs7422038 (differential imbalance p=2.5×10⁻⁰⁶). Each dot represents allelic imbalance in a single sample from breast (FIG. 1D; imbalance p=1.6×10⁻⁰⁶) and non-breast (FIG. 1E; imbalance p=0.17) cancers that are heterozygous for rs7422038, with approximate error bars estimated based on a binomial distribution of reads (i.e., sequence fragment). Data are presented as mean values +/−SE.

FIG. 2A-FIG. 2G is a series of line, bar, and heat map plots showing that cancer as-aQTLs are enriched for cancer risk heritability and eQTLs. FIG. 2A-FIG. 2C are a series of line plots showing LD score regression, demonstrating that as-aQTLs are strongly enriched for cancer risk heritability. The heritability enrichment increased when restricting the analysis to more significant as-aQTLs: FIG. 2A breast cancer (BRCA) as-aQTLs, FIG. 2B prostate cancer (PRAD) as-aQTLs, FIG. 2C renal cancer (KIRP) as-aQTLs. Data are presented as mean values +/−SE. FIG. 2D is a bar plot showing that a meta-analysis across all 7 cancer types showed significant enrichment of cancer risk heritability at as-aQTLs compared to all peaks, GTEx eQTLs, and coding regions. Data are presented as inverse-variance weighted mean values +/−SE. FIG. 1E is bar plot showing that as-aQTLs are most strongly enriched near transcription start sites. FIG. 2F is a heat map plot showing that the enrichments of cancer as-aQTLs for GTEx tissue eQTLs relative to all peaks. Non-significant enrichments (Z<2) are shown as NA. The most closely matching GTEx tissue-cancer pairs are framed in red. FIG. 2G is a bar plot showing that the enrichments of eQTLs at as-aQTLs/d-as-aQTLs are not specific to matching tissue-cancer pairs. Data are presented as mean values +/−SD.

FIG. 3A-FIG. 3E is a series of bar and dot plots showing that cancer as-aQTLs are associated with differential TF binding and gene expression. FIG. 3A is two bar plots showing that a series of 30 TF motifs for which the difference in motif scores between sequences with higher and lower allelic fractions was most significant. Allelic fractions are shown on the left and corresponding motif scores are on the right. Alleles with higher allelic fractions are shown in blue and alleles with lower allelic fractions in orange. Data are presented as mean values +/−SD. FIG. 3B is a dot plot showing that locations of motif-altering genetic variants inside as-aQTLs. FIG. 3C is a dot plot showing that locations of motif-altering genetic variants inside balanced peaks. FIG. 3D is a dot plot showing that the correlation between SNP PBS values and as-aQTL allelic fractions becomes very strong for large absolute SNP PBS values. Data are presented as Pearson correlations +/−SE. Number of pairs used for correlation analysis are shown above each point. FIG. 3E is a dot plot showing that the correlation between SuRE SNP ΔExpression_{ALT-REF} values and as-aQTL allelic fractions becomes very strong for highly significant SuRE SNP p-values. Data are presented as Pearson correlations +/−SE. Number of pairs used for correlation analysis are shown above each point.

FIG. 4A-FIG. 4D is a series of schematics and pie plots showing that regulome-Wide Association Study (RWAS) links accessible chromatin regions to cancer risk. FIG. 4A is a schematic showing that the RWAS results. FIG. 4B-FIG. 4D is three pie plots showing that the intersection between models with significant Bonferroni-corrected cross-validation p-values. FIG. 4B shows top1.total, top1.allelic and top1.combined models. FIG. 4C shows lasso.total, lasso.allelic and lasso.combined models. FIG. 4D shows Merged topl models (top1.total, top1.allelic and top1.combined) and merged lasso models (lasso.total, lasso.allelic and lasso.combined)

FIG. 5A-FIG. 5E is a series of bar, pie, and Manhattan plots showing that RWAS implicates hundreds of cancer risk mechanisms and outperforms TWAS. FIG. 5A is a bar plot showing that the numbers of significant RWAS associations discovered for 7 cancer types using 6 model types. FIG. 5B is a bar plot showing that the numbers of RWAS associations at known and novel risk loci that we discovered for each of the 7 cancer types across model types. FIG. 5C is two pie plots that show the discovered RWAS-significant peaks overlap most breast and prostate cancer risk loci, substantially more than TWAS-significant genes. FIG. 5D is a Manhattan plot showing breast cancer risk GWAS, RWAS, and TWAS. P-values are capped at 10⁻¹⁰⁰ FIG. 5E is a Manhattan plot showing prostate cancer risk GWAS, RWAS, and TWAS. P-values are capped at 10⁻¹⁰⁰.

FIG. 6A-FIG. 6B is a series of network analysis and box plots showing that breast cancer risk-associated RWAS peaks are linked to risk-associated TWAS genes. FIG. 6A is a series of network analysis plots that show correlations between significant TWAS gene and RWAS peak pairs at 30 breast cancer GWAS risk loci. Nodes representing TWAS genes are shown in red with gene names shown in brackets. Nodes representing RWAS peaks are shown in black. The color of the edges represents the strength of the correlations between models (absolute Pearson correlation). FIG. 6B is a box plot that show median absolute Pearson correlations between significant TWAS gene and RWAS peak pairs at each GWAS breast cancer risk locus. Horizontal lines inside the boxes indicate the medians. Box bounds show Q1 and Q3, whiskers are minima (Q1-1.5×(Q3-Q1)) and maxima (Q3+1.5×(Q3-Q1)).

FIG. 7A-FIG. 7C is schematic and two dot plots showing that allelic imbalance and RWAS can explain GWAS risk loci. FIG. 7A is a schematic showing that integrative model that uses allelic imbalance analysis and RWAS to connect TF motif alteration with cancer risk. FIG. 7B is a line plot showing that the RWAS-significant peak BRCA_124382 harbors the validated risk variant rs2981578 and explains a large portion of the GWAS signal in conditional analysis. FIG. 7C is a line plot showing that that show the RWAS-significant peak KIRC_1269 harbors the validated risk variant rs2992756 and explains a large portion of the GWAS signal in conditional analysis.

FIG. 8A-FIG. 8D is a of series dot, pie, and line plots showing the robust nature of the screening methods. FIG. 8A is a dot plot showing that principal component analysis confirming ancestry of the ATAC-Seq cancer sample subjects. FIG. 8B is a dot plot showing the results of downsampling, demonstrating the population-scale approach maximized statistical power with no sign of saturation. FIG. 8C is a pie plot showing pan-cancer as-aQTLs with and without MACS-called peaks. FIG. 8D is a line plot showing that the analysis conservatively controls for extreme somatic copy number alterations.

FIG. 9A-FIG. 9B is two bar plots showing cancer risk heritability of as-aQTLs. FIG. 9A is a bar plot showing fold enrichment for cancer risk heritability at corresponding cancer as-aQTLs and all other available annotations. FIG. 9B is a bar plot showing 5,000 most significant cancer type-specific as-aQTLs and all cancer type-specific peaks for 7 cancer types.

FIG. 10A-FIG. 10F is a series of line plots showing that as-aQTLs are strongly and specifically enriched for cancer risk heritability. FIG. 10A is a line plot showing that breast cancer type-specific as-aQTLs do not show heritability enrichment for non-cancer traits. FIG. 10B is a line plot showing that prostate cancer type-specific as-aQTLs do not show heritability enrichment for non-cancer traits. FIG. 10C is a line plot showing that renal cancer type-specific as-aQTLs do not show heritability enrichment for non-cancer traits. FIG. 10D is a line plot showing that breast cancer as-aQTLs display a significantly higher heritability enrichment than pan-cancer as-aQTLs. FIG. 10E is a line plot showing that prostate cancer as-aQTLs display a significantly higher heritability enrichment than pan-cancer as-aQTLs. FIG. 10F is a line plot showing that renal cancer as-aQTLs display a significantly higher heritability enrichment than pan-cancer as-aQTLs.

FIG. 11A-FIG. 11D is a series of heat maps and a dot plot showing enrichment of as-aQTLs and eQTLs at chromatin accessibility peaks. FIG. 11A is a heat map of GTEx tissue eQTLs showing as-aQTL enrichments relative to all peaks as background were higher than enrichments at all peaks relative to random genomic regions as background. FIG. 11B is a heat map of GTEx tissue eQTLs showing qualitatively that the majority of as-aQTLs likely lead to downstream effects on transcription. FIG. 11C is a heat map and FIG. 11D is a dot plot, both of GTEx tissue eQTLs showing eQTL enrichment at d-as-aQTLs.

FIG. 12A-FIG. 12L is a series of plots showing allelic fractions at TF motifs. FIG. 12A is a dot plot showing the Era1 TF motif. FIG. 12B is a dot plot showing the NF1 TF motif. FIG. 12C is a dot plot showing the ETV1 TF motif. FIG. 12D is a dot plot showing the FOXA1 TF motif. FIG. 12E is a dot plot showing the KLF1 TF motif. FIG. 12F is a dot plot showing the IRF1 TF motif. FIG. 12G is a dot plot showing the Tlx? TF motif. FIG. 12H is a dot plot showing the GRHL2 TF motif. FIG. 12I is a dot plot showing the ETS and RUNX TF motifs. FIG. 12J is a dot plot showing the HNF1b TF motif. FIG. 12K is a dot plot showing the p63 TF motif. FIG. 12L is a dot plot showing the USF1 TF motif.

FIG. 13A-FIG. 13C is a of series bar and dot plots showing allelic fractions at TF motifs. FIG. 13A is a bar plot showing that allelic fractions and HOMER motif scores at TF motifs. FIG. 13B and FIG. 13C are two dot plots showing the correlation between as-aQTL allelic fractions and TF binding/gene expression measurements.

FIG. 14A-FIG. 14F is a series of bar, pie, and box plots showing a comparison between RWAS and TWAS analyses between cancer types. FIG. 14A is a bar plot showing peaks with a genetic variant accounted for the majority of associations for 7 cancer types. FIG. 14B is a bar plot showing associated peaks that contained no variant. FIG. 14C is a bar plot showing breast and prostate cancer risk loci TWAS signal using GTEx RNA-seq. FIG. 14D is two pie plots showing that RWAS outperforms TWAS for breast and prostate cancer types. FIG. 14E and FIG. 14F are two box plots showing the correlation between TWAS gene and RWAS peak pairs.

FIG. 15A-FIG. 15B is a series of network analysis and box plots showing a comparison between RWAS and TWAS analysis for prostate cancer. FIG. 15A is a series of network analysis plots where the models are shown as nodes and the correlation are shown as edges between nodes for TWAS and RWAS analysis of prostate cancer. FIG. 15B is a box plot showing the correlation between TWAS gene and RWAS peak pairs for prostate cancer risk GWAS locus.

FIG. 16A-FIG. 16D is a series of pie and box plots showing a comparison between CWAS and RWAS analysis for prostate cancer. FIG. 16A is two pie plots that compare overlap of risk loci with RWAS-significant regions and CWAS-significant regions for prostate cancer. FIG. 16B, FIG. 16C, and FIG. 16D are three box plots showing the correlation between CWAS and RWAS peak pairs at prostate cancer risk loci.

FIG. 17A-FIG. 17F is a series of dot plots showing that variants near COSMIC genes have particularly strong evidence for causal cancer risk. FIG. 17A is a dot plot showing the KIRC_53002 peak at the TCRF7L2 gene region. FIG. 17B is a dot plot showing the UCEC_76834 peak at the RAD51B gene region. FIG. 17C is a dot plot showing the ESCA_110366 peak at the SETBP1 gene region. FIG. 17D is a dot plot showing the THCA_68825 peak at the AKT1 gene region. FIG. 17E is a dot plot showing the LIHC_61297 peak at the EXT1 gene region. FIG. 17F is a dot plot showing the MESO_4104 peak at the FAM46C gene region.

### DETAILED DESCRIPTION OF THE DISCLOSURE

### Definitions

Unless defined otherwise, all technical and scientific terms used herein have the same meaning as is commonly understood by one of skill in art to which the subject matter herein belongs. As used in the specification and the appended claims, unless specified to the contrary, the following terms have the meaning indicated in order to facilitate the understanding of the present disclosure.

As used in the description and the appended claims, the singular forms “a”, “an”, and “the” include plural referents unless the context clearly dictates otherwise. Thus, for example, reference to “a composition” includes mixtures of two or more such compositions, reference to “an inhibitor” includes mixtures of two or more such inhibitors, and the like.

Unless stated otherwise, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value.

The term “approximately” as used herein refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless otherwise clear from context, all numerical values provided herein are modified by the term “about.”

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed disclosure.

As used herein, the term “diagnosing” refers to classifying a pathology, symptom, disease, or disorder, determining a severity of the pathology (e.g., grade or stage), monitoring pathology progression, forecasting an outcome of pathology, and/or determining prospects of recovery.

By the terms “effective amount” and “therapeutically effective amount” of a formulation or formulation component is meant a sufficient amount of the formulation or component, alone or in a combination, to provide the desired effect. For example, by “an effective amount” is meant an amount of a compound, alone or in a combination, required to ameliorate the symptoms of a disease, e.g., cancer, relative to an untreated patient. The effective amount of active compound(s) used to practice the present disclosure for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

The term “gene region” as used herein refers to a region of the genome in which at least one gene, or an open reading frame, resides and the sequence around that gene. The terms “gene” and “open reading frame” as used interchangeably herein to refer to a distinct nucleic acid sequence forming part of a chromosome, the order of which determines the order of monomers in a polypeptide or nucleic acid molecule which a cell may synthesize. Typically, a gene will have a plurality of exons (on average 12 exons), with an average exon length of about 250 base pairs. Each exon is typically separated by an intron, with an average intron length of about 5,500 base pairs. The total region occupied by a single gene (including exons and introns) is on average about 55,000 base pairs. A gene region encompasses a gene and a region upstream and downstream of the gene, typically the length of a gene within the gene region, or on average about 50,000 base pairs (50 kilobase pairs; 50 Kp) before and after the gene. The upstream region begins 50 Kb before the transcriptional starting point of the gene in the gene region. The downstream region begins at the transcriptional ending point of the gene and extends for 50 Kb. These upstream and downstream regions typically include transcription factor (TF) binding motifs (i.e., sites in which a TF binds DNA), a regulatory region containing regulator elements, and a promoter region containing one or more promoters.

The term “promoter” as used herein refers to a nucleic acid sequence that regulates, directly or indirectly, the transcription of a corresponding nucleic acid open reading frame sequence to which it is operably linked, which in the context of the present disclosure, is typically a gene or an oncogenic gene. A promoter may function alone to regulate transcription, or it may act in concert with one or more other regulatory sequences (e.g., enhancers or silencers, or regulatory elements that may be present in the gene region or in found in an expression vector). Promoters are located near the transcription start sites of open reading frames, on the same strand and upstream on the DNA (towards the 5′ region of the sense strand). Promoters typically range from about 100-1000 base pairs in length. Methods of Use

In some aspects, the present disclosure is directed to methods of treating, diagnosing, qualifying, and/or determining risk of a disease or disorder in a subject with the use of the inventive biomarkers described herein. In some embodiments, the disease or disorder is neoplasia. In some embodiments, the disease or disorder is cancer. In some embodiments, the cancer is breast cancer, prostate cancer, colorectal cancer, renal cancer, glioma, lung cancer, or melanoma.

A “disease” is generally regarded as a state of health of a subject wherein the subject cannot maintain homeostasis, and if the disease is not ameliorated then the subject's health continues to deteriorate. In contrast, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health. In some embodiments, compounds of the application may be useful in the treatment of proliferative diseases and disorders (e.g., cancer or benign neoplasms). As used herein, the term “cell proliferative disease or disorder” refers to the conditions characterized by unregulated or abnormal cell growth, or both. Cell proliferative disorders include noncancerous conditions, precancerous conditions, and cancer. In one aspect, the present disclosure is directed to a method of determining whether a subject is at risk of developing or will develop cancer. The method entails obtaining a test sample from the subject at risk of having cancer, determining the presence of a biomarker in the test sample, wherein the biomarker is selected from the group consisting of the biomarkers set forth in Table 8A-Table 8G, determining that the subject is at risk of developing neoplasia or cancer based on the presence of the biomarker in the test sample.

The term “subject” (or “patient”) as used herein includes all members of the animal kingdom prone to or suffering from the indicated disease or disorder. In some embodiments, the subject is a mammal, e.g., a human or a non-human mammal. The methods are also applicable to companion animals such as dogs and cats as well as livestock such as cows, horses, sheep, goats, pigs, and other domesticated and wild animals. A subject “in need of” treatment according to the present disclosure may be “suffering from or suspected of suffering from” a specific disease or disorder may have been positively diagnosed or otherwise presents with a sufficient number of risk factors or a sufficient number or combination of signs or symptoms such that a medical professional could diagnose or suspect that the subject was suffering from the disease or disorder. Thus, subjects suffering from, and suspected of suffering from, a specific disease or disorder are not necessarily two distinct groups.

In some embodiments, the presence or significant increase of a biomarker in a subject relative to a reference identifies the subject as having an increased likelihood of developing a disease or disorder. In cases where the subject is more likely to develop a disease or disorder (e.g., breast cancer), a cancer therapy may be administered to that subject, as described herein. Biomarkers

One aspect of the present disclosure is the use of biomarkers in methods of treating, diagnosing, qualifying, and/or determining risk of a disease or disorder in a subject. The term “biomarker” as used herein refers to molecular indicators of a specific biological property, a biochemical feature, or facet that can be used to determine the presence or absence and/or severity of a particular disease or disorder. In the present disclosure, the term “biomarker” may refer to any suitable analyte, for example, a nucleic acid sequence, a polypeptide, expression product, or a measurable reaction by an expression product, and combinations or fragments thereof. Biomarkers may have a changed value in a subject as compared to a reference. In some embodiments, the biomarker comprises a variant as compared to a reference. A variant may include nucleic acids (e.g., DNA) which differs in its nucleic acid sequence. A variant may include polypeptides which differ in its amino acid sequence. Variants may be allelic variants, splice variants, or any other species-specific homologs, paralogs, or orthologs.

In the working examples described herein, the biomarkers are genetic variants (e.g., single nucleotide polymorphism) in a nucleic acid molecule (e.g., a patient's genome or a patient's tumor genome). In some embodiments, the biomarker is a genetic variant and is detected by its presence (e.g., through sequencing). In some embodiments, the biomarker is a single nucleotide polymorphism (SNP). An SNP may be referred to by an rsID number, which is a unique number used to identify a specific SNP. An artisan skilled in the art would recognize an rsID and understand that it is the most common naming convention used for the vast majority of SNPs by researchers and the relevant databases. The artisan skilled in the art would also know that information relating to an SNP identified by a rsID number is freely available and maintained by the National Library of Medicine in the Single Nucleotide Polymorphism Database (dbSNP) of Nucleotide Sequence Variation. The dbSNP database includes SNP-specific information regarding the SNP's position in the genome, the alleles present (the reference nucleotide in a so-called wild-type condition and the altered nucleotide), the frequency at which the altered nucleotide has previously been detected, and its type. Furthermore, a skilled artisan would recognize alternative SNP identification systems including genome build and chromatin position identifiers. The entries for each biomarker listed in Table 8A-Table 8G include (i) the rsID number for each biomarker (column labeled ‘RSID’), (ii) the chromosome on which the SNP is found (column labeled ‘CHR’), (iii) the position of the SNP on that chromosome (column labeled ‘VAR.P1’), (iv) the reference (so called ‘wild-type’) nucleotide (column labeled ‘REF’), and (v) the altered nucleotide (column labeled ‘ALT’), as well as additional information described herein.

The term “single nucleotide polymorphism” (SNP) as used herein refers to a polymorphic site occupied by a single nucleotide, which is the site of variation between allelic sequences. The site is usually preceded by and followed by highly conserved sequences of the allele (e.g., sequences that vary in less than 1/100 or 1/1000 members of a population). A SNP usually arises due to substitution of one nucleotide for another at the polymorphic site. SNPs can also arise from a deletion of a nucleotide or an insertion of a nucleotide relative to a reference allele. Typically, the polymorphic site is occupied by a base other than the reference base. For example, where the reference allele contains the base ‘T’ (thymidine) at the polymorphic site, the altered allele can contain a “C” (cytidine), “G” (guanine), or “A” (adenine) at the polymorphic site. SNP's may occur in protein-coding nucleic acid sequences, in which case they may give rise to a defective or otherwise variant protein, or genetic disease. Such an SNP may alter the coding sequence of the gene and therefore specify another amino acid (a “missense” SNP), or a SNP may introduce a stop codon (a “nonsense” SNP). When a SNP does not alter the amino acid sequence of a protein, the SNP is called “silent.” SNP's may also occur in noncoding regions of the nucleotide sequence. This may result in defective protein expression, e.g., as a result of alternative spicing, alteration of a regulatory region (which may contain one or more regulatory elements), or a transcription factor binding motif, or the SNP may have no effect on the function of the protein.

The term “nucleic acid molecule” as used herein refers to DNA molecules (e.g., cDNA or genomic DNA) and RNA molecules (e.g., mRNA) and analogs of the DNA or RNA generated using nucleotide analogs. The nucleic acid molecule can be single-stranded or double-stranded.

The biomarkers of the present disclosure are measured from a sample obtained from a subject. The term “sample” as used herein refers to a portion of a subject. A sample may be whole blood, plasma, serum, saliva, urine, stool (e.g., feces), tears, or any other suitable bodily fluid. A sample may also be a tissue sample (e.g., a biopsy) such as a lymph node, breast sample, small intestine, colon sample, prostate sample, or surgical resection tissue. In some embodiments, a method of the present disclosure may further include obtaining a sample from a subject prior to detecting or determining the presence or level of a biomarker in the sample. In some embodiments, the sample is obtained from a healthy tissue (e.g., non-cancerous). In other embodiments, the sample is obtained from a cancerous tissue (e.g., a biopsy). In some embodiments, the sample is taken from a patient who has already developed cancer. In some cases, the sample is taken from a patient who has not been diagnosed cancer, the patient may have been identified as being at risk of a cancer, as known in the art.

Once a sample has been obtained, a biomarker value may be measured. The term “biomarker value” refers to a value measured or derived for at least one corresponding biomarker of a subject and which is at least partially indicative of a sequence (e.g., a SNP), concentration, expression level, peak intensity, or chromatin accessibility of the biomarker. Thus, the biomarker values could be measured biomarker values, which are values of biomarkers measured from the subject, or alternatively could be derived biomarker values, which are values that have been derived from one or more measured biomarker values, for example by applying a function to the one or more measured biomarker values.

Biomarker values can be of any appropriate form depending on the manner in which the values are determined. For example, the biomarker values could be determined using high-throughput technologies such as mass spectrometry, sequencing platforms, array and hybridization platforms, immunoassays, flow cytometry, or any combination of such technologies. In some embodiments, the biomarker values relate to presence of a genetic variant in a subject's DNA (e.g., a SNP). In some embodiments, the biomarker value is detected or measured by sequencing, genotyping, or polymerase chain reaction (PCR). Genotyping techniques may include restriction fragment length polymorphism identification (RFLPI), random amplified polymorphic detection (RAPD), amplified fragment length polymorphism detection (AFLPD), polymerase chain reaction (PCR), allele specific oligonucleotide (ASO) probes, and hybridization to DNA microarrays or DNA beads. In some embodiments, the biomarker value is a presence or absence of a sequence. If a genetic variant is present, e.g., in the subject's genome, then the biomarker is said to be present. Alternatively, if a genetic variant is not present, then the biomarker is said to be absent.

In some embodiments, the biomarker values relate, directly or indirectly, to the chromatin accessibility in a region of a subject's DNA. In other embodiments, the biomarker values relate to a level of activity or abundance of an expression product or other measurable molecule, quantified using known techniques. In some cases, the biomarker values may be in the form of amplification amounts, or cycle times, which are a logarithmic representation of the concentration of the biomarker within a sample.

Typically, a biomarker will be compared to a reference to determine if the biomarker is present or altered. The terms “reference” and “control” are used interchangeably herein to refer to a sample containing the one or more biomarkers of interest from a control subject or tissue. Typically, biomarkers in a reference have been quantified and the value or range thereof is known for subject without the disease, disorder, or condition (i.e., a normal range). The reference may be from a sample from an individual or from multiple subjects, typically in the form of an average value, or average range. The reference sample may be a biological sample or data obtained from a previously harvested biological sample.

In some embodiments, the biomarker is predictive of a subject developing a cancer. In some of these embodiments, the test sample a biomarker is measured from a non-cancerous sample, for example, blood, serum, plasma, urine, stool, or a non-cancerous tissue sample (i.e., cells). In some embodiments, the test sample is a cancerous sample, for example a tumor biopsy, or a bodily fluid containing cancerous cells (e.g., blood, serum, plasma, urine, or stool containing cancer cells).

In some embodiments, the biomarker is located within a regulatory region. The term “regulatory region” is used herein to refer the region of DNA where RNA polymerase and other accessory transcription modulator proteins bind the DNA and interact to control RNA synthesis. In some embodiments, the biomarker is located within a promoter region. In some embodiments, the biomarker is located within a TF binding motif. In some embodiments, the TF binding motif in which the biomarker is located is a binding motif for a family member of the Runt, Ets, APETALA 2 (AP2), basic leucine zipper (bZIP), zinc-finger (Zf), or E2 Factor (E2F) families.

In some embodiments, the biomarker is within a TF binding motif, or is a genetic variant that disrupts a TF binding motif. In some embodiments, the TF binding motif is an activating protein-1 (AP-1), AP-2α, AP-2γ, activating transcription factor 3 (ATF3), BTB domain and CNC homolog 1 (BACH1), BACH2, basic leucine zipper ATF-like transcription factor (BATF), cone-rod homeobox (CRX), E2f transcription factor 1 (E2F1), E2F3, E2F4, E2F6, ETS homologous factor (EHF), E74-like ETS transcription factor 1 (ELF1), ELF4, Ets proto-oncogene 1 (ETS1), ETS variant transcription factor 4 (ETV4), FAFB, fli-1 proto-oncogene, ETS transcription factor (FLI1), Fos proto-oncogene, AP-1 transcription factor subunit (FOS), FOS Like 1, AP-1 transcription factor subunit (FOSL1, also called FRA1), FOSL2 (also called FRA2), forkhead box 1 (FOXO1), heart and neural crest derivatives expressed 2 (HAND2), Jun proto-oncogene, AP-1 transcription factor subunit (JUN), JunB proto-oncogene, AP-1 transcription factor subunit (JUNB), kruppel-like factor 4 (KLF4), KLF14, maf bzip transcription factor A (MAFA), MAFK, nanog homeobox (NANOG), Nuclear factor, erythroid 2 (NF-E2), nfe2-like bzip transcription factor 2 (NFE2L2 also called NRF2), POU class 1 homeobox 1 (POUIF1 also called PIT1), paired-like homeodomain 1 (PITX1), recombination signal binding protein for immunoglobulin kappa J region (RBPJ), RUNKX1, (SCL), sine oculis homeobox homolog (SIX2), SAM pointed domain containing ETS transcription factor (SPDEF), spi-1 proto-oncogene (SPI1, also called PU.1), SP2, SP5, TAL BHLH transcription factor 1 (TAL1, also called SCL), zinc finger protein 263 (ZNF263), ZNF467, or ZNF675 binding motif.

In some embodiments, the TF binding motif is KLF14, PIT1, RBPJ, RUNX1, SP2, SP5, ZNF263, or ZNF467.

In some embodiments, the biomarker selected from the group of the biomarkers set forth within Table 8A, and the subject has or is at risk of having breast cancer. In some of these embodiments, the biomarker is rs2981578, rs11599804, rs10787473, rs12258200, rs1316014, rs1314913, rs62090606, rs249473, rs2494734, rs1462985, rs2992756, or rs3767812. In some embodiments, the biomarker is rs2981578 or rs2992756.

In some embodiments, the biomarker selected from the group within Table 8A, and the subject has or is at risk of having breast cancer. In some embodiments, the biomarker is selected from the group of the biomarkers set forth in Table 8B, and the subject has or is at risk of having prostate cancer. In some embodiments, the biomarker is selected from the group of the biomarkers set forth in Table 8C, and the subject has or is at risk of having colorectal cancer. In some embodiments, the biomarker is selected from the group of the biomarkers set forth in Table 8D, and the subject has or is at risk of having renal cancer. In some embodiments, the biomarker is selected from the group of the biomarkers set forth in Table 8E, and the subject has or is at risk of having glioma. In some embodiments, the biomarker is selected from the group of the biomarkers set forth in Table 8F, and the subject has or is at risk of having lung cancer. In some embodiments, the biomarker is selected from the group of the biomarkers set forth in Table 8G, and the subject has or is at risk of having melanoma.

### Neoplasia

In some aspects, the present disclosure is directed to treating a cancer in a subject. The method entails obtaining a test sample from the subject at risk of having cancer, determining the presence of a biomarker in the test sample, wherein the biomarker is selected from the group consisting of the biomarkers set forth in Table 8A-Table 8G, determining that the subject is at risk of developing cancer based on the presence of the biomarker in the test sample, and administering to the subject an effective amount of one or more cancer therapies. The term “neoplasia” as used herein refers to a disease or disorder characterized by excess proliferation or reduced apoptosis. In some embodiments, the neoplasia is a benign growth. In some embodiments the neoplasia is cancerous. Illustrative neoplasms for which the invention can be used include, but are not limited to breast cancer, prostate cancer, renal cancer, colorectal cancer, glioma, lung cancer, melanoma, pancreatic cancer, adrenocortical carcinoma (ACC), bladder urothelial carcinoma (BLCA), breast invasive carcinoma (BRCA), cervical squamous cell carcinoma (CESC), cholangiocarcinoma (CHOL), colon adenocarcinoma (COAD), esophageal carcinoma (ESCA), glioblastoma multiforme (GBM), head and neck squamous cell carcinoma (HNSC), kidney renal clear cell carcinoma (KIRC), kidney renal papillary cell carcinoma (KIRP), low grade glioma (LGG), liver hepatocellular carcinoma (LIHC), lung adenocarcinoma (LUAD), lung squamous cell carcinoma (LUSC), mesothelioma (MESO), pheochromocytoma and paraganglioma (PCPG), prostate adenocarcinoma (PRAD), skin cutaneous melanoma (SKCM), stomach adenocarcinoma (STAD), testicular germ cell tumors (TGCT), thyroid carcinoma (THCA), and uterine corpus endometrial carcinoma (UCEC).

In some embodiments, a subject has a neoplasm or is at risk for developing a neoplasm. One or more genes may be causally linked to a neoplasm or the development of a neoplasm. In some embodiments, a subject has a cancer or is at risk for developing a cancer. Cancer causal genes are described in the catalogue of somatic mutations in cancer (COSMIC), which records known somatic mutations found in cancer patients. COSMIC is maintained by the Cancer Genome Project and the Sanger Institute and is freely available to the public.

In general, the inventive methods of treating, diagnosing, qualifying, and/or determining risk a disease may include administering to the subject in need thereof an effective amount of one or more therapies. The term “effective amount” as used herein refers to a sufficient amount of a cancer therapy to provide the desired effect. Thus, the term “effective amount” includes the amount of a cancer therapy to that, when administered, induces a positive modification in the disease or disorder to be treated, or is sufficient to prevent development or progression of the disease or disorder, or alleviate to some extent, one or more of the symptoms of the disease or disorder being treated in a subject, or which simply kills or inhibits the growth of diseased (e.g., neoplasia or cancer) cells. In some embodiments, the therapy is an anti-cancer therapeutic approved for treating one or more cancers. In some embodiments, the therapy is surgery to remove one or more tumors, radiation therapy, chemotherapy, hormone therapy, targeted therapy, immunotherapy, or a combination thereof.

### Cancers and Therapies Therefore

In some embodiments, a subject has breast cancer or is at risk for developing breast cancer. Breast cancer is a group of cancers in which cells in the breast grow out of control, and include breast cancer, a precancer or precancerous condition of the breast, benign growths or lesions of the breast, hyperplasia, metaplasia, and dysplasia of the breast, and invasive ductal carcinoma and invasive lobular carcinoma.

Breast cancer treatments may include surgery, radiation therapy, chemotherapy, hormone therapy, targeted therapy, and immunotherapy. Surgery may include sentinel lymph node biopsy, breast-conserving surgery, total mastectomy (simple mastectomy), or modified radical mastectomy.

Hormone therapy removes hormones or blocks hormone signaling pathways, thereby stopping cancer cell growth and proliferation. Hormones are substances made by glands in the body and circulated in the bloodstream. Cancer cells often produce hormones that promote tumor growth. Hormone therapy to treat breast cancer may include ovarian ablation, tamoxifen, luteinizing hormone-releasing hormone (LHRH) agonists, aromatase inhibitors, megestrol acetate (Megace®) and anti-estrogen therapy such as fulvestrant (Faslodex®), or combinations thereof. Ovarian ablation may involve surgery (oophorectomy), radiation therapy, or medication therapy. Medication therapy may include goserelin (Zoladex®), tamoxifen (Soltamox®), and leuprolide (Lupron®).

Breast cancer prevention and therapeutics, that are suitable for the combination with the inventive methods described herein, may also include raloxifene and tamoxifen citrate (Soltamox®), abemaciclib (Verzenio®), paclitaxel (Abraxane®), ado-trastuzumab emtansine (Kadcyla®), everolimus (Afinitor®, Zortress®, Afinitor Disperz®), alpelisib (Piqray®), anastrozole (Arimidex®), pamidronate disodium (ArediaR), exemestane (Aromasin®), cyclophosphamide, doxorubicin hydrochloride, epirubicin hydrochloride (Ellence®), fam-trastuzumab deruxtecan-nxki (Enhertu®), fluorouracil (5-FU; Adrucil®), toremifene (Fareston®), letrozole (Femara®), gemcitabine (Gemzar®, Infugem®), eribulin mesylate (Halaven®), trastuzumab and hyaluronidase-oysk (Herceptin Hylecta®), trastuzumab (Herceptin®), palbociclib (Ibrance®), ixabepilone (Ixempra®), pembrolizumab (Keytruda®), ribociclib (Kisqali®), olaparib (Lynparza®), margetuximab-cmkb (Margenza®), neratinib maleate (Nerlynx®), pertuzumab (Perjeta®), pertuzumab trastuzumab and hyaluronidase-zzxf (Phesgo®), talazoparib tosylate (Talzenna®), docetaxel (TaxotereR), atezolizumab (Tecentriq®), thiotepa (Tepadina®), methotrexate sodium (Trexall®), sacituzumab govitecan-hziy (Trodelvy®), tucatinib (Tukysa®), lapatinib ditosylate (Tykerb®), vinblastine sulfate, capecitabine (Xeloda®), and goserelin acetate (Zoladex®).

In some embodiments, a subject has prostate cancer or is at risk for developing prostate cancer. Prostate cancer is a cancer that occurs in the prostate. Treatments for prostate cancer may include watchful waiting, surgery, radiation therapy, radiopharmaceutical therapy, chemotherapy, hormone therapy, targeted therapy, immunotherapy, bisphosphonate therapy, cryosurgery, high-intensity-focused ultrasound therapy, proton beam radiation therapy, and photodynamic therapy. Surgeries for prostate cancer may include radical prostatectomy, pelvic lymphadenectomy, and transurethral resection of the prostate (TURP).

Hormone therapy for prostate cancer may include abiraterone acetate, estrogens, LHRH agonists, antiandrogens, orchiectomy, or a combination thereof. Abiraterone acetate can prevent prostate cancer cells from making androgens. Estrogen treatment can prevent the testicles from making testosterone. LHRH agonists can stop the testicles from making testosterone; examples of which may include leuprolide, goserelin, and buserelin. Antiandrogens can block the action of androgens, such as testosterone; examples of which may include flutamide, bicalutamide, enzalutamide, apalutamide, nilutamide, and darolutamide. Orchiectomy is a surgical procedure to remove one or both testicles, the main source of male hormones, such as testosterone.

Prostate cancer therapeutics, that are suitable for the combination with the inventive methods described herein, include abiraterone (Yonsa®, Zytiga®), bicalutamide (Casodex®), leuprolide (Eligard®, Lupron Depot®), apalutamide (Erleada®), degarelix (Firmagon®), flutamide, cabazitaxel (Jevtana®), lutetium Lu 177 vipivotide tetraxetan (Pluvicto®), Olaparib (Lynparza®), mitoxantrone, nilutamide (Nilandron®), darolutamide (Nubeqa®), relugolix (Orgovyx®), sipuleucel-T (Provenge®), radium 223 Dichloride (Xofigo®), rucaparib camsylate (Rubraca®), docetaxel (TaxotereR), enzalutamide (Xtandi®), and goserelin (Zoladex®).

In some embodiments, a subject has colorectal cancer or is at risk for developing colorectal cancer. Colorectal cancer is cancer that starts in the colon or the rectum. Treatments for colorectal cancer may include surgery, radiofrequency ablation, cryosurgery, chemotherapy, radiation therapy, targeted therapy, and immunotherapy.

Colon cancer surgeries may include local excision, resection of the colon with anastomosis, polypectomy, and resection of the colon with colostomy.

Targeted therapeutics to treat colorectal cancer may include monoclonal antibodies, angiogenesis inhibitors, protein kinase inhibitors, or combinations thereof. Suitable monoclonal antibodies include antibodies against vascular endothelial growth factor (VEGF) or epidermal growth factor receptor (EGFR). Angiogenesis inhibitors may also be used as a targeted therapeutic and include ziv-aflibercept and regorafenib.

Colorectal cancer therapeutics, that are suitable for the combination with the inventive methods described herein, include bevacizumab-maly (Alymsys®), bevacizumab (Avastin®, Mvasi®, Zirabev®), irinotecan (Camptosar®), Ramucirumab (Cyramza®), oxaliplatin (Eloxatin®), cetuximab (Erbitux®), 5-FU (Adrucil®), ipilimumab (Yervoy®), pembrolizumab (Keytruda®), leucovorin, trifluridine and tipiracil hydrochloride (Lonsurf®), nivolumab (Opdivo®), regorafenib (Stivarga®), panitumumab (Vectibix®), capecitabine (Xeloda®), and ziv-aflibercept (Zaltrap®).

In some embodiments, a subject has renal cancer or is at risk for developing renal cancer. Renal cancer, also called hypernephroma, renal adenocarcinoma, and kidney cancer, involves cancerous growths in the tubules and tissues of the kidney. Treatments for renal cancer may include surgery, radiation therapy, chemotherapy, immunotherapy, and targeted therapy. Renal cancer surgeries may include partial nephrectomy, simple nephrectomy, and radical nephrectomy.

Renal cancer therapeutics, that are suitable for the combination with the inventive methods described herein, include everolimus (Afinitor®, Zortress®, Afinitor Disperz®), bevacizumab-maly (Alymsys®), bevacizumab (Avastin®, Mvasi®, Zirabev®), avelumab (Bavencio®), cabozantinib-S-malate (Cabometyx®), tivozanib (Fotivda®), IL-2, aldesleukin (Proleukin), axitinib (Inlyta®), pembrolizumab (Keytruda®), lenvatinib mesylate (Lenvima®), sorafenib tosylate (Nexavar®), nivolumab (Opdivo®), sunitinib malate (Sutent®), temsirolimus (Torisel®), pazopanib (Votrient®), belzutifan (Welireg®), and ipilimumab (Yervoy®).

In some embodiments, a subject has glioma or is at risk for developing glioma. Glioma is a cancer of the brain and spinal cord. Three common types of gliomas are astrocytomas (including astrocytoma, anaplastic astrocytoma and glioblastoma), ependymomas (including anaplastic ependymoma, myxopapillary ependymoma and subependymoma), and oligodendrogliomas (including oligodendroglioma, anaplastic oligodendroglioma and anaplastic oligoastrocytoma). Treatments for glioma may include active surveillance, surgery, radiation therapy, chemotherapy, and targeted therapy.

Glioma treatments, that are suitable for the combination with the inventive methods described herein, include everolimus (Afinitor®, Zortress®, Afinitor Disperz®), bevacizumab-maly (Alymsys®), bevacizumab (Avastin®, Mvasi®, Zirabev®), carmustine (BiCNUR), naxitamab-gqgk (Danyelza®), carmustine implant (Gliadel Wafer®), lomustine, temozolomide (Temodar®), and belzutifan (Welireg®).

In some embodiments, a subject has lung cancer or is at risk for developing lung cancer. Lung cancer is cancer that forms in tissues of the lung, usually in the cells lining air passages. Lung cancers usually are grouped into two main types, small cell and non-small cell (including adenocarcinoma and squamous cell carcinoma). The treatment of lung cancer depends on the type of lung cancer. For non-small cell lung cancer, treatments may include surgery, radiation therapy, chemotherapy, targeted therapy, immunotherapy, laser therapy, photodynamic therapy (PDT), cryosurgery, and electrocautery. Surgery to treat non-small cell lung cancer may include wedge resection, lobectomy, pneumonectomy, or sleeve resection.

Treatments for small cell lung cancer may include surgery, chemotherapy, radiation therapy, immunotherapy, laser therapy, and endoscopic stent placement.

Lung cancer therapeutics, that are suitable for the combination with the inventive methods described herein, include paclitaxel albumin-stabilized nanoparticle formulation (Abraxane®), everolimus (Afinitor®, Zortress®, Afinitor Disperz®), alectinib (Alecensa®), pemetrexed disodium (Alimta®), brigatinib (Alunbrig®), bevacizumab (Alymsys®, Mvasi®, Avastin®, Zirabev®), amivantamab-vmjw (Rybrevant®), Ramucirumab (Cyramza®), doxorubicin hydrochloride, mobocertinib succinate (Exkivity®), pralsetinib (Gavreto®), afatinib dimaleate (Gilotrif®), gemcitabine (Gemzar®, Infugem®), durvalumab (Imfinzi), gefitinib (Iressa®), pembrolizumab (Keytruda®), cemiplimab-rwlc (Libtayo®), lorlatinib (Lorbrena®), sotorasib (Lumakras®), trametinib dimethyl sulfoxide (Mekinist®), nivolumab (Opdivo®), necitumumab (Portrazza®), selpercatinib (Retevmo®), Entrectinib (Rozlytrek®), capmatinib (Tabrecta®), dabrafenib mesylate (Tafinlar®), osimertinib mesylate (Tagrisso®), erlotinib (Tarceva®), docetaxel (Taxotere®), atezolizumab (Tecentriq®), tepotinib Hydrochloride (Tepmetko®), methotrexate (Trexall®), dacomitinib (Vizimpro®), vinorelbine tartrate, crizotinib (Xalkori®), ipilimumab (Yervoy®), and ceritinib (Zykadia®).

In some embodiments, a subject has melanoma or is at risk for developing melanoma. Melanoma is a cancer that usually starts in a certain type of skin cell, i.e., melanocytes. Melanoma is also called malignant melanoma and cutaneous melanoma. Melanoma treatments may include immunotherapy, biologic therapy, radiation therapy, or chemotherapy. Melanoma surgeries may include excisional biopsies or complete surgical excision.

Chemotherapy agents for melanoma treatment include temozolomide, dacarbazine (also termed DTIC), immunotherapy (with interleukin-2 (IL-2) or interferon (IFN)), as well as local perfusion, ipilimumab, pembrolizumab, and nivolumab, BRAF inhibitors, such as vemurafenib and dabrafenib, and trametinib.

Additional melanoma drugs, that are suitable for the combination with the inventive methods described herein, include encorafenib (BraftoviR), cobimetinib fumarate (Cotellic®), dacarbazine, talimogene haherparepvec (Imlygic®), recombinant Interferon Alfa-2b (Intron AR), pembrolizumab (Keytruda®), tebentafusp-tebn (Kimmtrak®), trametinib dimethyl sulfoxide (Mekinist®), binimetinib (Mektovi®), nivolumab (Opdivo®), nivolumab and relatlimab-rmbw (Opdualag®), peginterferon Alfa-2b (PEG-Intron®, Sylatron®), aldesleukin (Proleukin®), dabrafenib mesylate (Tafinlar®), ipilimumab (Yervoy®), and vemurafenib (Zelboraf®).

### Transcription Factor-Mediated Cancers

In some embodiments, a subject has a transcription factor-mediated cancer or is at risk for developing a transcription factor-mediated cancer. In some embodiments, a biomarker identified herein alters a transcription factor (TF) binding motif. A biomarker representing an altered TF binding motif may guide the therapy a subject receives after identification of such a biomarker. In some embodiments, the biomarker is a genetic variation that disrupts a TF binding motif, resulting in altered TF binding to a subject's DNA. In some embodiments, the genetic variation decreases TF binding to the TF binding motif as compared to a TF binding motif without a genetic variation. In some embodiments, the genetic variation completely blocks TF binding. In other embodiments, the genetic variation increases TF binding as compared to a TF binding motif without a genetic variation. Altered TF binding may mediate (e.g., cause or contribute to) a disease or disorder in the subject. In these cases, additional therapies may be administered to treat the TF-mediated disease or disorder. The therapy to treat a TF-mediated disease or disorder may act directly on the affected TF or on genes or proteins regulated by the TF.

In some embodiments, the disrupted TF motif (e.g., a ZNF467 motif) increases the risk for prostate cancer and a therapy as described herein may be administered. In some embodiments, the disrupted TF motif (e.g., POU1F1, RBPJ, RUNX1, SP2, SP5, ZNF263, or ZNF467 motifs) increases the risk for Uterine corpus endometrial carcinoma (UCEC), and a therapy to treat UCEC is administered.

UCEC therapies may include surgery, radiation therapy, chemotherapy, hormone therapy, and targeted therapy. Surgeries for UCEC may include total hysterectomy, radical hysterectomy, bilateral salpingo-oophorectomy, omentectomy, or lymph node dissection.

Targeted therapy for treating UCEC may include monoclonal antibodies, mTOR inhibitors, or signal transduction inhibitors. Representative monoclonal antibodies for the treatment of UCEC include the angiogenesis inhibitor bevacizumab (Alymsys®, Avastin®, Mvasi®, Zirabev®). Representative mTOR inhibitors are everolimus (Zortress®, Afinitor Disperz®, Afinitor®) and ridaforolimus. Signal transduction inhibitors include metformin (Fortamet®, Glumetza®).

In some embodiments, the disrupted TF motif is a POU class 1 homeobox transcription factor 1 (POU1F1, also known as PIT1) binding motif, and increases the risk for melanoma, colorectal cancer, or UCEC. In these embodiments, a therapy directed thereto may be administered. Additional therapeutics to treat POU1F1-mediated diseases or disorders may be administered, including bromocriptine (Parlodel®, Cycloset®), buspirone (BuSpar®), carbamazepine (Tegretol®, Tegretol XR®, Epitol®), clonidine (Kapvay®), clozapine (Clozaril®, FazaClo ODTR, Versacloz®), dexamethasone (Ozurdex®, Maxidex®), diethylstilbestrol, dopamine (Intropin®, Dopastat®, Revimine®), human growth hormone analogs (e.g., somatropin (Genotropin®, Humatrope®), mecasermin (Increlex®); see, e.g., Fuh et al., J Biol Chem 270:13133-7 (1995)), naloxone (Narcan®), or progesterone (Crinone®, Prochieve®, Prometrium®).

In some embodiments, the disrupted TF motif (e.g., a SP5 motif) increases the risk for hepatocellular carcinoma, gastric cancer, or colon cancer and a therapy directed thereto may be administered. In some embodiments, the disrupted TF motif (e.g., a SP2 motif) increases the risk for gastrointestinal cancer and a therapy directed thereto may be administered.

Gastrointestinal cancer treatments may include surgery, endoscopic mucosal resection, chemotherapy, radiation therapy, chemoradiation, targeted therapy, and immunotherapy. Surgeries for gastrointestinal cancer may include subtotal gastrectomy and total gastrectomy.

Targeted therapy to treat gastrointestinal cancer may include monoclonal antibodies and multikinase inhibitors. Representative monoclonal antibodies include trastuzumab (Herceptin®) and ramucirumab (Cyramza®). Angiogenesis inhibitors may be used to treat gastrointestinal cancer and may include trastuzumab and ramucirumab. A representative multikinase inhibitor include regorafenib (Stivarga®).

Additional gastrointestinal cancer therapeutics include ramucirumab (Cyramza®), doxorubicin hydrochloride, fam-trastuzumab deruxtecan-nxki (Enhertu®), fluorouracil injection (5-FU), trastuzumab (Herceptin®), pembrolizumab (Keytruda®), trifluridine and tipiracil hydrochloride (Lonsurf®), mitomycin, nivolumab (Opdivo®), docetaxel (Taxotere®), and trastuzumab.

In some embodiments, the disrupted TF motif is a motif for RUNX1 binding, and increases the risk for renal cancer, breast cancer, lung cancer, gastrointestinal cancer, and acute myeloid leukemia (AML). In these embodiments, a therapy directed thereto may be administered. AML treatment may include chemotherapy, chemotherapy combined with stem cell transplant, radiation therapy, targeted therapy, and other drug therapies.

Targeted therapy for AML may include administration of monoclonal antibodies, protein kinase inhibitors, and signal transduction inhibitors. Representative monoclonal antibodies for the treatment of AML include midostaurin (Rydapt®, Tauritmo®), gilteritinib (Xospata®), glasdegib, ivosidenib, and enasidenib. Representative signal transduction inhibitors include Glasdegib (Daurismo®), ivosidenib (Tibsovo®), and enasidenib (Idhifa®).

Additional AML therapeutics include arsenic trioxide, all-trans retinoic acid (ATRA), daunorubicin hydrochloride (Cerubidine®), cyclophosphamide, cytarabine, daunorubicin hydrochloride, daunorubicin hydrochloride and Cytarabine Liposome, glasdegib (Daurismo®), dexamethasone, doxorubicin, enasidenib mesylate (Idhifa®), idarubicin (Idamycin PFS®), mitoxantrone, gemtuzumab ozogamicin (Mylotarg®), azacitidine (Onureg®), prednisone, daunorubicin (Rubidomycin®), midostaurin (Rydapt®), thioguanine (Tabloid®), ivosidenib (Tibsovo®), arsenic trioxide (Trisenox®), venetoclax (venclexta®), vincristine sulfate, daunorubicin hydrochloride and cytarabine liposome (Vyxeos®), and gilteritinib fumarate (Xospata®).

Additional therapeutics to treat RUNX1-mediated diseases or disorders may be administered, including administering anisomycin, azacitidine, cinobufagin, cytarabine, daunorubicin (Cerubidine®), disulfiram (Antabuse®), doxorubicin (Adriamycin®, Lipodox®, Lipodox 50®, Doxil®), fenbendazole, imatinib (Gleevec®), plerixafor (Mozobil®), the proteolysis-targeting chimera (PROTAC) ARV-825, or narciclasine.

In some embodiments, the disrupted TF motif is a RBPJ binding motif, and increases the risk for lung cancer or testicular cancer. In these embodiments, a therapy directed thereto may be administered.

Testicular cancer may be treated with surgery, radiation therapy, chemotherapy, surveillance, or high-dose chemotherapy with stem cell transplant. Surgeries for testicular cancer include inguinal orchiectomy. Additional testicular cancer therapeutics include bleomycin sulfate, cisplatin, dactinomycin (Cosmegen®), dactinomycin, Etoposide (Etopophos®), ifosfamide (Ifex®), and vinblastine sulfate.

In some embodiments, the disrupted TF motif is a Kruppel like factor 14 (KLF14) binding motif, and a disrupted KLF14 TF motif may increase the risk for melanoma, lung cancer, ovarian cancer, or breast cancer. In these embodiments, a therapy directed thereto may be administered.

Additional therapeutics to treat KLF14-mediated diseases or disorders may be administered, including administering bavachinin, GW9662, perhexiline, perhexiline maleate (Prexsig®), pioglitazone (Actos®), or rosiglitazone (Avandia®).

### Immunotherapy

In some embodiments, the additional anti-cancer agent includes immunotherapy, e.g., immune checkpoint inhibitors. Representative examples of immune checkpoint molecules that may be targeted by the additional therapy include PD-1, PDL1, CTLA4, KIR, TIGIT, TIM-3, LAG-3, BTLA, VISTA, CD47, and NKG2A. Clinically available examples of immune checkpoint inhibitors include durvalumab (Imfinzi®), atezolizumab (Tecentriq®), and avelumab (Bavencio®). Clinically available examples of PD-1 inhibitors include nivolumab (Opdivo®), pembrolizumab (Keytruda®), and cemiplimab (Libtayo®).

### Chemotherapy

Anti-cancer therapies also include a variety of combination therapies with both chemical and radiation-based treatments. Combination chemotherapies include, for example, Abraxane®, altretamine, docetaxel, Herceptin®, methotrexate, Novantrone®, Zoladex®, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomy emcitabinetabin, mitomycin, etoposide (VP16), tamoxifen, raloxifene, estrogen receptor binding agents, Taxol®, gemcitabine, Navelbine®, farnesyl-protein tansferase inhibitors, transplatinum, 5-fluorouracil, vincristine, vinblastine and methotrexate, or any analog or derivative variant of the foregoing and also combinations thereof.

### Radiotherapy

Anti-cancer therapies also include radiation-based, DNA-damaging treatments. Combination radiotherapies include what are commonly known as gamma-rays, X-rays, and/or the directed delivery of radioisotopes to cancer cells which cause a broad range of damage on DNA, on the replication and repair of DNA, and on the assembly and maintenance of chromosomes. Dosage ranges for radioisotopes vary widely, and depend on the half-life of the isotope, the strength and type of radiation emitted, and the uptake by the neoplastic cells and will be determined by the attending physician.

### Combination Therapy

The therapies used to treat the disease and disorders diagnosed by the methods of the present disclosure may be used in combination with at least one other active agent, e.g., anti-cancer agent or regimen, in treating diseases and disorders. The term “in combination” in this context means that the agents are co-administered, which includes substantially contemporaneous administration, by the same or separate dosage forms, or sequentially, e.g., as part of the same treatment regimen or by way of successive treatment regimens. Thus, if given sequentially, at the onset of administration of the second therapy, the first of the two therapies is, in some cases, still detectable at effective concentrations at the site of treatment. The sequence and time interval may be determined such that they can act together (e.g., synergistically to provide an increased benefit than if they were administered otherwise). For example, the therapeutics may be administered at the same time or sequentially in any order at different points in time; however, if not administered at the same time, they may be administered sufficiently close in time so as to provide the desired therapeutic effect, which may be in a synergistic fashion. Thus, the terms are not limited to the administration of the active agents at exactly the same time.

The dosage of the two therapeutics may be the same or even lower than known or recommended doses of the two therapeutics alone. See, Hardman et al., eds., Goodman & Gilman's The Pharmacological Basis of Therapeutics, 10th ed., McGraw-Hill, New York, 2001; Physician's Desk Reference 60th ed., 2006. Agents and therapeutics that may be used in combination are known in the art. See, e.g., U.S. Pat. No. 9,101,622 (Section 5.2 thereof). Representative examples of additional active agents and treatment regimens are disclosed elsewhere herein.

Representative examples of additional active agents and treatment regimens include radiation therapy, chemotherapeutics (e.g., mitotic inhibitors, angiogenesis inhibitors, anti-hormones, autophagy inhibitors, alkylating agents, intercalating antibiotics, growth factor inhibitors, anti-androgens, signal transduction pathway inhibitors, anti-microtubule agents, platinum coordination complexes, HDAC inhibitors, proteasome inhibitors, and topoisomerase inhibitors), immunomodulators, therapeutic antibodies (e.g., mono-specific and bispecific antibodies) and CAR-T therapy. In some embodiments, the treatment regimen may include immunotherapy. In some embodiments, the immunotherapy is a checkpoint inhibitor.

In some embodiments, the therapeutic used in combination a second therapeutic, which may be administered less than 5 minutes apart, less than 30 minutes apart, less than 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours apart. The two or more additional therapeutics may be administered within the same patient visit.

In some embodiments, two or more additional therapeutics are cyclically administered. Cycling therapy involves the administration of one therapeutic for a period of time, followed by the administration of a second therapeutic for a period of time and repeating this sequential administration, i.e., the cycle, in order to reduce the development of resistance to one or both of the therapeutics, to avoid or reduce the side effects of one or both of the therapeutics, and/or to improve the efficacy of the therapies. In one example, cycling therapy involves the administration of a first therapeutic for a period of time, followed by the administration of a second therapeutic for a period of time, optionally, followed by the administration of a third therapeutic for a period of time and so forth, and repeating this sequential administration, i.e., the cycle in order to reduce the development of resistance to one of the therapeutics, to avoid or reduce the side effects of one of the therapeutics, and/or to improve the efficacy of the therapeutics.

To demonstrate the feasibility of this disclosure, several exemplary embodiments will be shown in detail herein.

### EXAMPLES

### Example 1: Materials and Methods

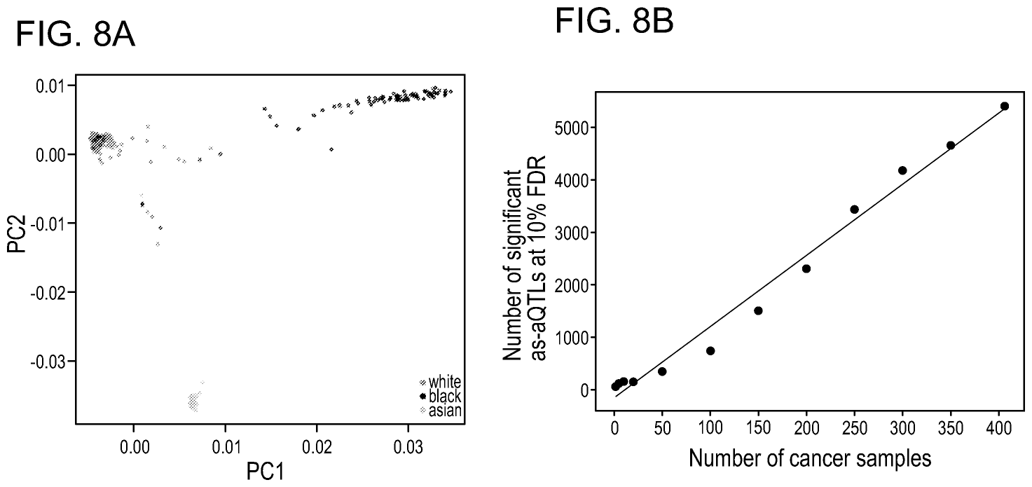

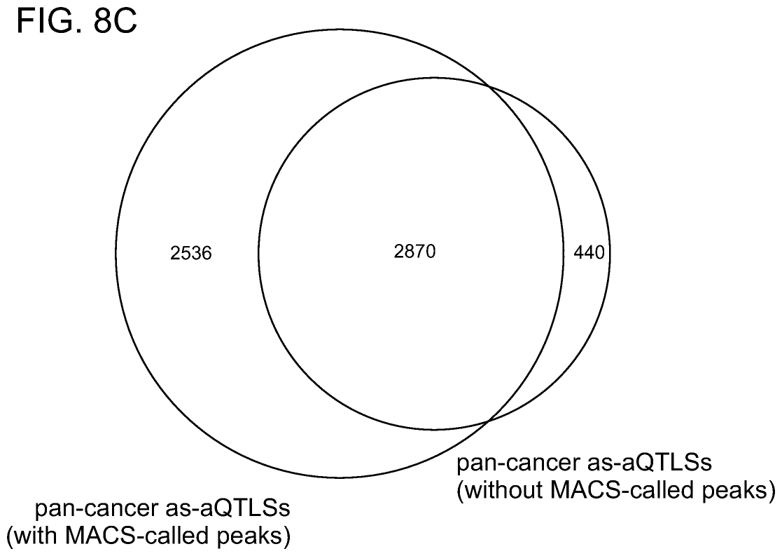

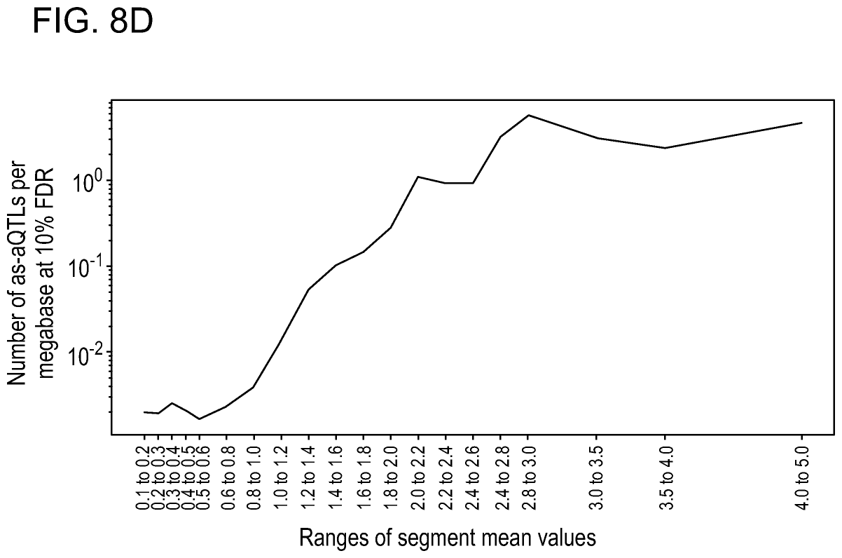

Data preparation for allelic imbalance analysis. BAM files for 406 ATAC-Seq cancer samples were retrieved from the Cancer Genome Atlas (TCGA) Genomic Data Commons Data Portal (Corces, M. R. et al., Science 362, (2018)). The reported ethnicities of the individuals to whom the samples belong were as follows: Asian (48), black or African American (72), white (222), and not reported (64). 212 samples were male, and 194 samples were female. Ancestry was confirmed through principal component analysis (FIG. 8A). All samples were elected to include in the analysis because allelic imbalance is not inflated by population structure due to “canceling out” of trans/environmental effects by comparing functional activity at the two alleles within an individual, and only testing heterozygous carriers (thus not biased by population differences in allele frequency).

For each sample, the sequencing reads were remapped to the hg19 reference genome (human_glk_v37.fasta). Corresponding 400 germline genotyping files were retrieved from TCGA and imputed using the IMPUTE2 algorithm on the Sanger Imputation Server (imputation.sanger.ac.uk) using the same hg19 reference and the Haplotype Reference Consortium reference panel (McCarthy, S. et al., Nat. Genet. 48, 1279-1283 (2016)). The BAM and VCF files were then processed using the WASP allele-specific pipeline for unbiased read mapping and molecular QTL discovery (van de Geijn, B. et al., Nat. Methods 12, 1061-1063(2015)) and the number of allele-specific reads at each imputed SNP in each BAM file was determined. Pan-cancer and cancer type-specific peak sets were also obtained that were previously called with MACS2 (Zhang, Y. et al., Genome Biol. 9, R137 (2008)) from the Genomic Data Commons (GDC) and lifted them to the hg19 reference genome. The utilized peak calling method normalized peak significance scores across samples and cancer types to account for differences in sequencing depth, sample quality, and the number of samples per cancer type (Corces, M. R. et al., Science 362, (2018)). Peaks were not centered at variant positions.

Alternative peak definitions for as-aQTL discovery. An alternative peak definition was investigated by dividing the genome into adjacent 500 bp regions and repeating the pan-cancer analysis. Using these uniformly defined peaks, 2870 out of 5406 imbalanced regions were identified that were found previously in the pan-cancer analysis that relied on formal peaks called by the MACS algorithm (Zhang, Y. et al., Genome Biol. 9, R137 (2008)), and only 440 new as-aQTLs (FIG. 8C). This suggests that the imbalance analysis does not strongly depend on peak definitions and power is maximized when using conventionally defined peaks.

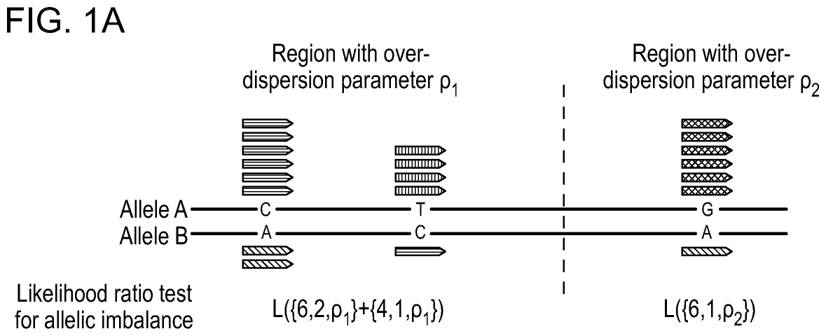

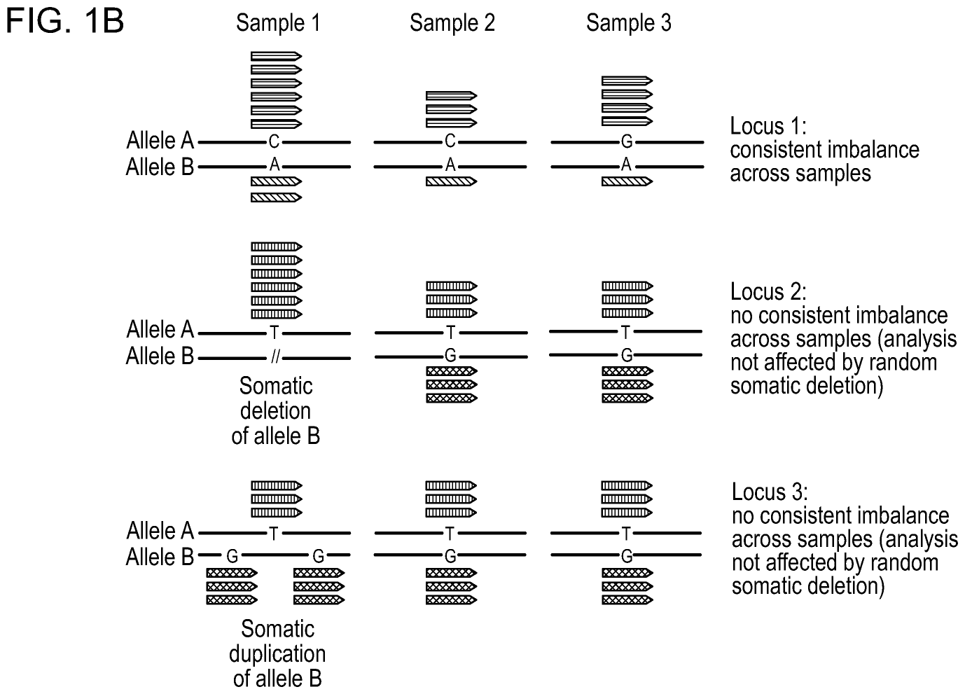

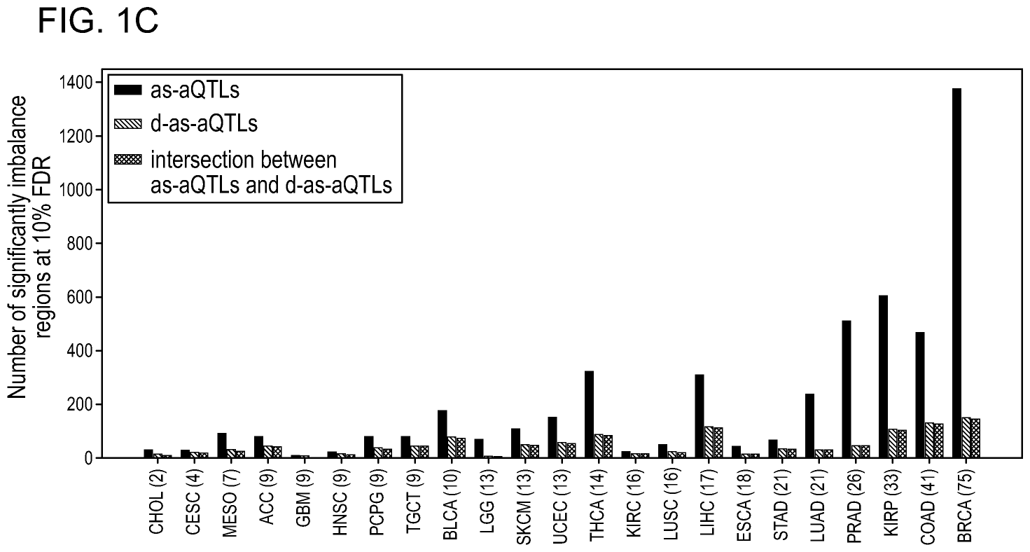

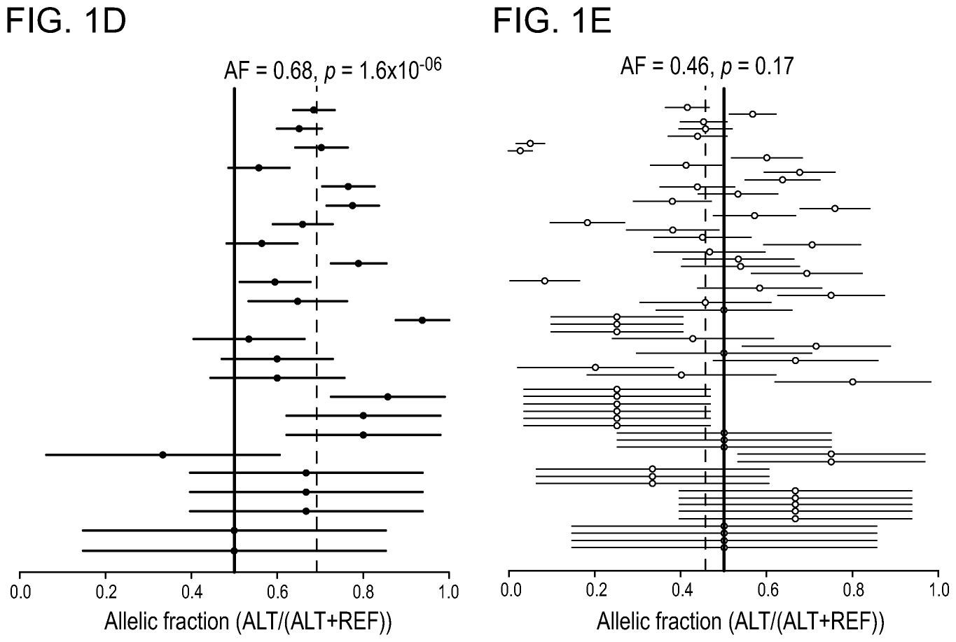

Germline cis-regulatory variants influencing chromatin accessibility in cancers were identified and characterized. To maximize power while accounting for cancer type-specific effects, allelic imbalance in ATAC-seq was measured across many individuals as a readout of population-level germline variant activity. Allele-specific analyses pose unique challenges, particularly their vulnerability to false-positive associations when the read distribution is not modeled properly. Multiple studies have shown that molecular sequence reads have greater variability than expected from a binomial distribution (van et al., Nat Methods 12:1061-3 (2015); Kumasaka et al., Nat Genet 48:206-13 (2016)). In cancer data, this variability is additionally strongly dependent on local somatic copy number alterations (Van et al., Proc Natl Acad Sci U S A 107:16910-5 (2010)). The recently developed stratified Allele-Specific (stratAS) was utilized test to account for these potential biases (Gusev, A. et al., bioRxiv 631150 (2019)). This approach models the read distribution as a beta-binomial to account for overdispersion in sequencing reads due to technical factors. Additionally, overdispersion parameters are inferred for each sample and each local copy number variation (CNV) region, to account for overdispersion induced by somatic excess of one haplotype across the region (FIG. 1A). Importantly, the allele-specific signal is aggregated across the population of samples to identify consistent allelic effects in the population, rather than estimated within each sample (as is commonly done with allele-specific DNA analyses to identify individual somatic alterations).

Random somatic CNVs will amplify/delete each allele arbitrarily and are thus not expected to produce allele consistent imbalance across multiple samples (FIG. 1B) except in rare cases with unusually high CNV induced coverage. For this study, the potential impact of extreme CNVs were investigated and conservatively filtered out regions with extreme CNVs from the analysis (FIG. 8A). Finally, variance in cancer sample purity does not bias the discovery of allele-specific signals because it impacts both alleles equally and was not considered, though lower overall purity may influence the sensitivity to detect cancer type-specific as-aQTLs.

Examination of CNV effects on as-aQTL discovery. To guard against potential contamination from extreme CNVs, the as-aQTLs discovery was conducted after filtering out genomic regions in individual cancer samples that had CNV mean values of >0.6 (segment mean=|log2(CNV/2|). This threshold approximately corresponds to the gain or loss of up to one allele and it excluded ˜5% of the genome from the imbalance analysis. This cutoff was determined after conducting an analysis that assessed the impact of somatic CNVs on as-aQTL discovery. It was found that as-aQTLs are strongly enriched in extremely high-CNV genomic regions despite correcting sequencing read overdispersion using local parameters for each sample and each local CNV region (FIG. 8D). Because the model disclosed herein corrected for read overdispersion at CNVs, the observed allelic imbalance is likely reflective of true differential chromatin accessibility and may have a complex germline-somatic relationship to the tested SNP. For example, a locus with one individual having extremely high coverage allelic imbalance due to a CNV, and with all other individuals exhibiting low coverage but allelic consistency with the CNV carrier is difficult to definitively assign to the CNV or a cis germline variant or both. Therefore, a conservative choice was made to only include regions that do not harbor extreme CNVs in the as-aQTL analysis.

Allelic imbalance analysis. Tests for allele-specificity were carried out using the stratAS software (Gusev, A. et al., bioRxiv 631150 (2019)), which uses a beta-binomial distribution to model the local overdispersion of sequencing reads. To calculate local over-dispersion parameters, CNV data was used that was available for 401 of 406 ATAC-Seq cancer samples. For the remaining 5 samples, a single global overdispersion parameter was calculated. Variants that had a minimum of 5 mapped sequencing reads were used to compute the overdispersion parameters. For each of the 23 cancer types, the stratAS allelic imbalance analysis was conducted at 500 bp pan-cancer ATAC-Seq peaks (lifted to hg19) while excluding regions with a CNV segment mean value of >0.6. The window size was set to 250 to test only heterozygous variants within 500 bp peaks. Minimum required coverage per heterozygous variant was kept at the default setting of 1. No samples were excluded from the analysis.

Cancer type-specific as-aQTLs were identified by including only samples from each cancer type in the analysis. Differentially imbalanced regions (d-as-aQTLs) for each cancer type were identified by testing in each cancer type for a difference in allelic fraction relative to samples from all the other 22 cancer types (by a likelihood ratio test implemented in stratAS). Pan-cancer imbalanced regions were identified by analyzing all cancer samples together. The (differential) imbalance p-values output by stratAS were used to identify significant as-aQTLs/d-as-aQTLs at 10% FDR. Only ˜1.3% of pan-cancer as-aQTL variants were located inside ENCODE blacklisted regions (Table 2X), significantly less than the fraction of the genome that is covered by ENCODE blacklisted regions (˜10%), and these were therefore not excluded from the analysis.

Cancer risk heritability enrichment analysis. To conduct risk heritability enrichment analysis, intersected as-aQTLs was first identified in each cancer type with the corresponding cancer type-specific peaks, to ensure direct comparability between the two. This was done because the imbalance analysis was conducted using the pan-cancer peak set which led to the identification of some cancer type-specific as-aQTLs at positions with no peak in the given cancer type. Next, as-aQTLs identified in each cancer type was ordered by the statistical significance of the imbalance (from most to least significant). Since many as-aQTLs contained more than one genetic variant within the tested 500 bp window, the variant closest to the center of the peak was selected and Bonferroni-corrected its p-value by dividing it by the number of variants in the as-aQTL. Stratified LD score regression (LDSC) analysis was conducted using the baselineLD_v2.2 (Houlahan, K. E. et al., Nat. Med. 25, 1615-1626 (2019)) annotations as background and publicly available GWAS summary statistics for 7 cancer types and matching cancer type-specific as-aQTLs identified in the stratAS analysis (plus pan-cancer as-aQTLs).

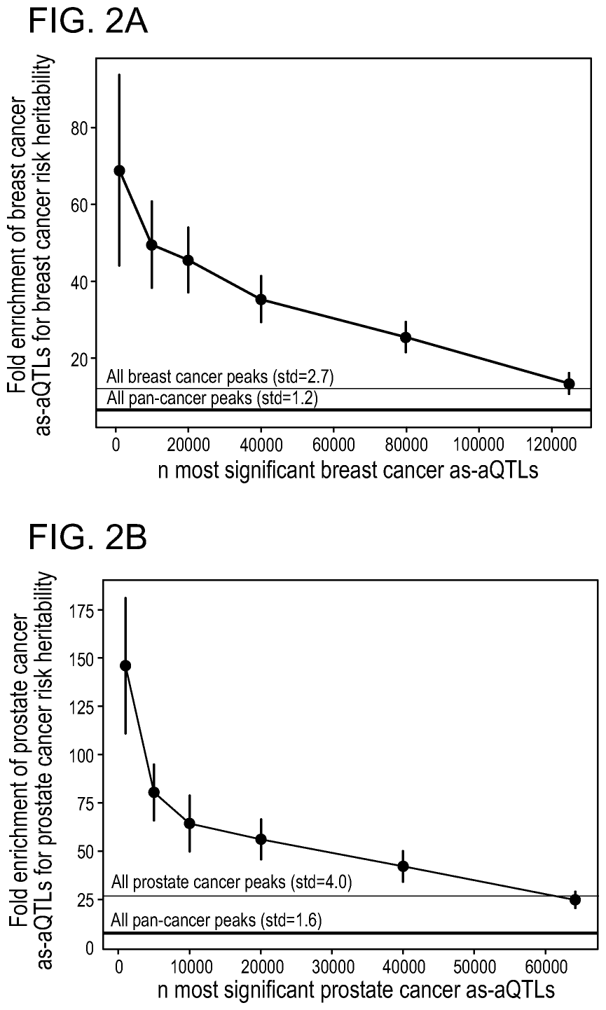

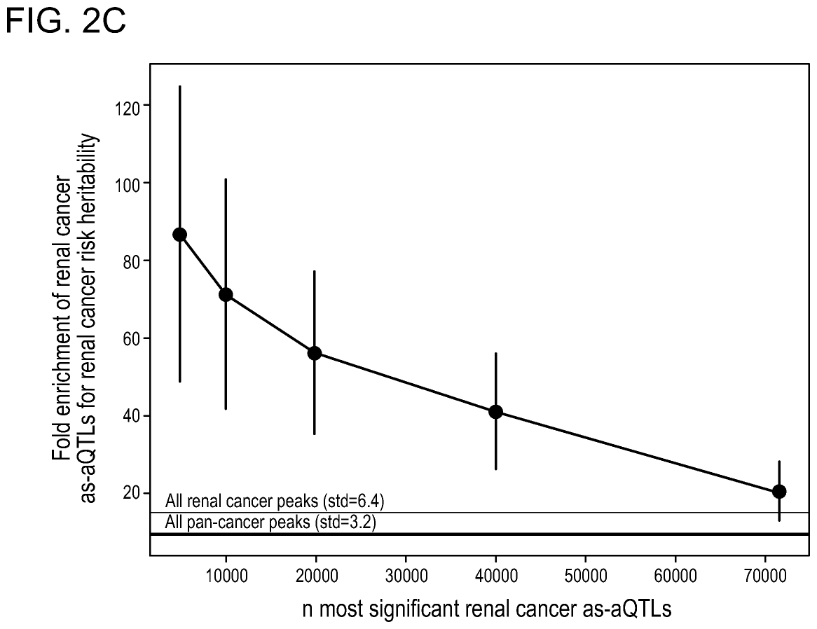

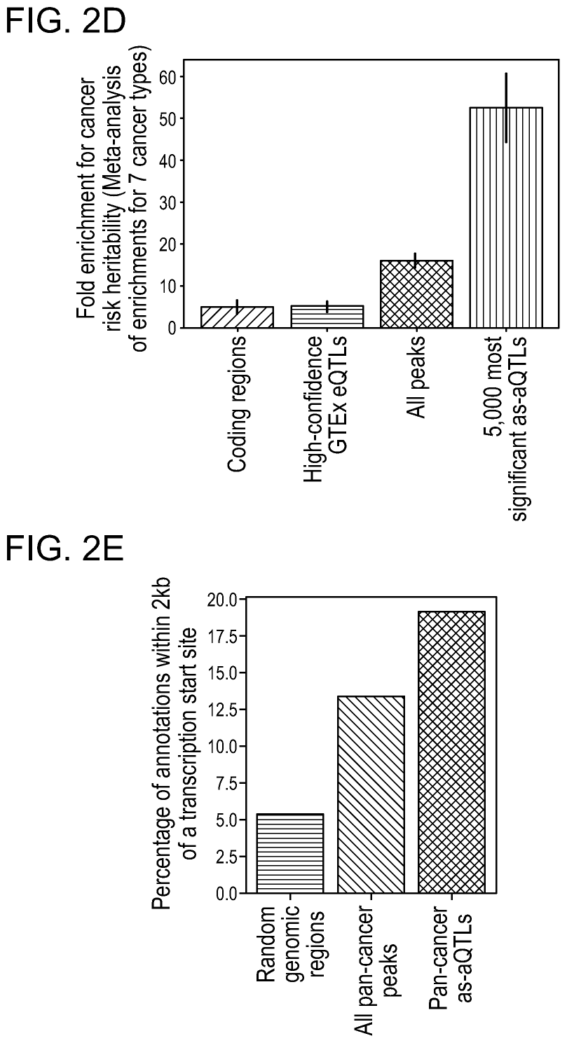

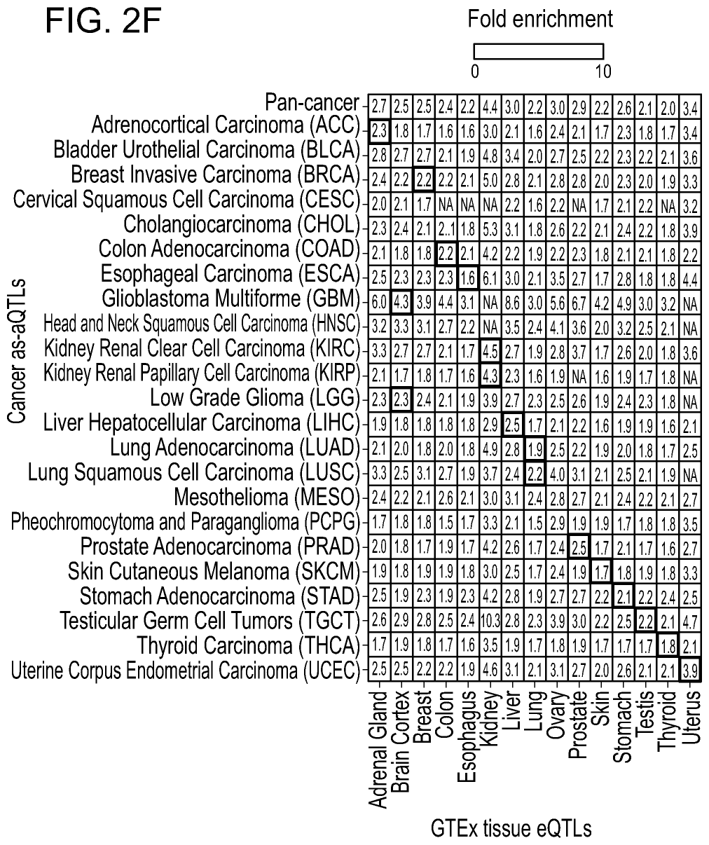

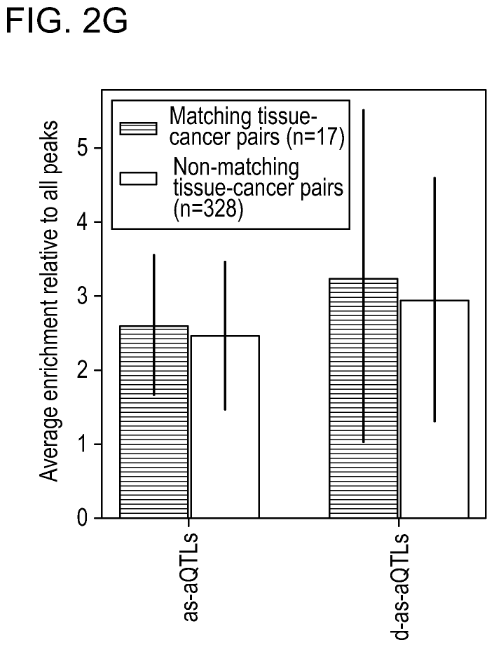

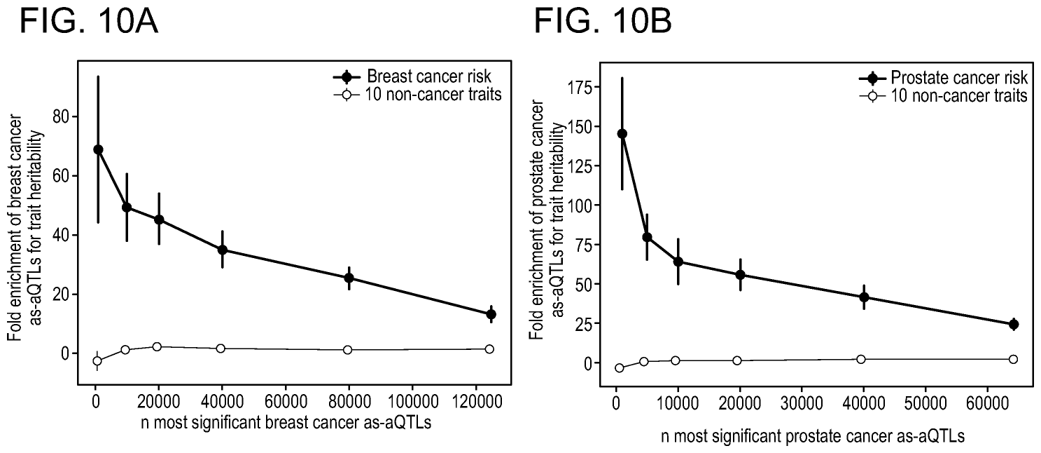

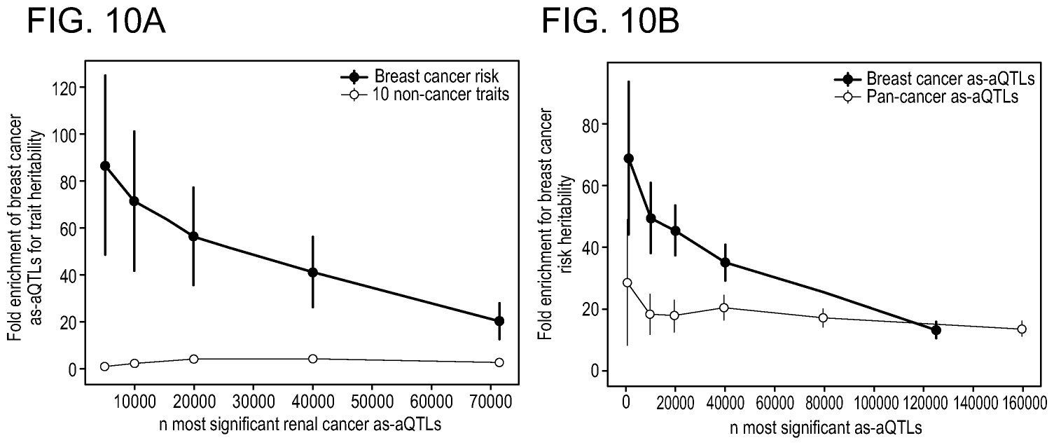

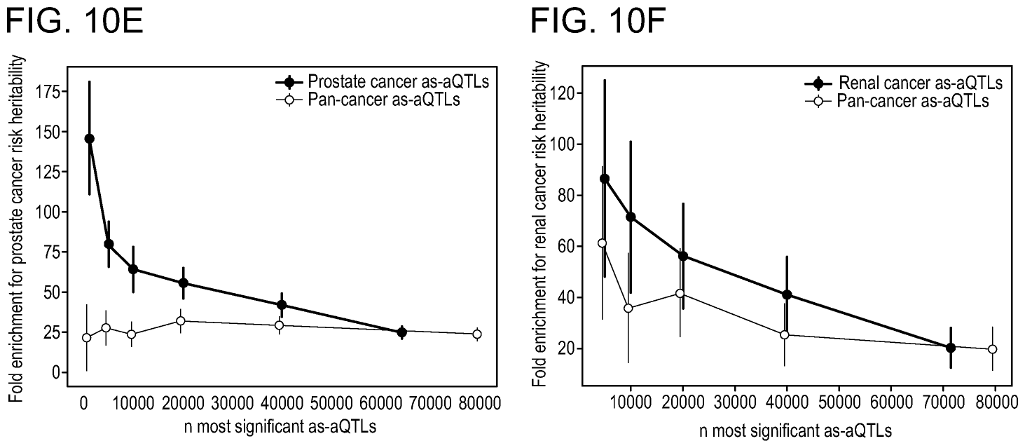

The eQTL annotation was part of baselineLD_v2.2 and was derived from prior probabilistic fine-mapping of eQTLs and shown to be the state-of-the-art for estimating heritability explained by eQTLs by modeling the uncertainty about the true functional SNP in the region (Hormozdiari, F. et al., Nat. Genet. 50, 1041-1047 (2018)). When more than one cancer type in the ATAC-Seq dataset matched a GWAS summary statistic, the cancer type with more samples/as-aQTLs was chosen. In particular, the Kidney Renal Papillary Cell Carcinoma (KIRP) was used over Kidney Renal Papillary Cell Carcinoma (KIRC); Lung Adenocarcinoma (LUAD) was used over Lung Squamous Cell Carcinoma (LUSC), and Low-Grade Glioma (LGG) was used over Glioblastoma Multiforme (GBM). To generate the curves shown in FIG. 2A-FIG. 2G and FIG. 10A-FIG. 10F, the LDSC analysis was repeated using different peak sets as an input annotation: from the peak set containing 1,000 most significant as-aQTLs to a peak set containing all cancer-specific peaks. The meta-analysis of the LDSC enrichments was done using inverse variance weighting of the 7 cancer type-specific enrichment analyses. To examine heritability enrichment at breast, prostate, and kidney cancer as-aQTL peaks for non- cancer traits (FIG. 10A-FIG. 10C), the heritability estimates of 10 traits from the UK Biobank (cholesterol, education years, hypertension, type 2 diabetes, height, BMI, neuroticism, Vitamin D level, asthma, and erythrocyte count) was averaged using inverse variance weighting. For the enrichment analysis of top GWAS variants, 5,000 of the most significant as-aQTLs and 3,000 most significant risk variants were determined for each cancer type. These numbers were chosen such that there is enough overlap between the annotations but also minimizing the number of non-significant as-aQTLs/risk variants. The enrichment of risk variants at as-aQTLs relative to random genomic sequences were calculated as background based on multiple samplings.

Expression QTL enrichment analysis. Expression QTLs (eQTLs) identified in GTEx Analysis v8 (GTEx Consortium. Science 369, 1318-1330 (2020)) were retrieved for 15 for tissue types that most closely match the 23 cancer types in the ATAC-Seq dataset. The enrichment of eQTLs were calculated at significant as-aQTLs (10% FDR) for each of 360 tissue-cancer pairs (incl. pan-cancer as-aQTLs). To generate a background distribution that accounted for the expected enrichment of eQTLs in generic peaks, an equivalent number of cancer type-specific peaks were sampled and overlapped with GTEx tissue-specific eQTLs. Finally, the enrichment of as-aQTLs were calculated relative to all peaks for GTEx tissue-specific eQTLs, as well as z-scores based on multiple samplings. The enrichments at d-as-aQTLs were calculated in the same way. The calculation of eQTL enrichments at all cancer type-specific peaks was done in the same way but used random genomic regions as background. The enrichment analysis for cancer eQTLs was conducted in the same way using eQTLs that were discovered in TCGA cancer samples (Gong, J. et al., 46, D971-D976 (2018)).

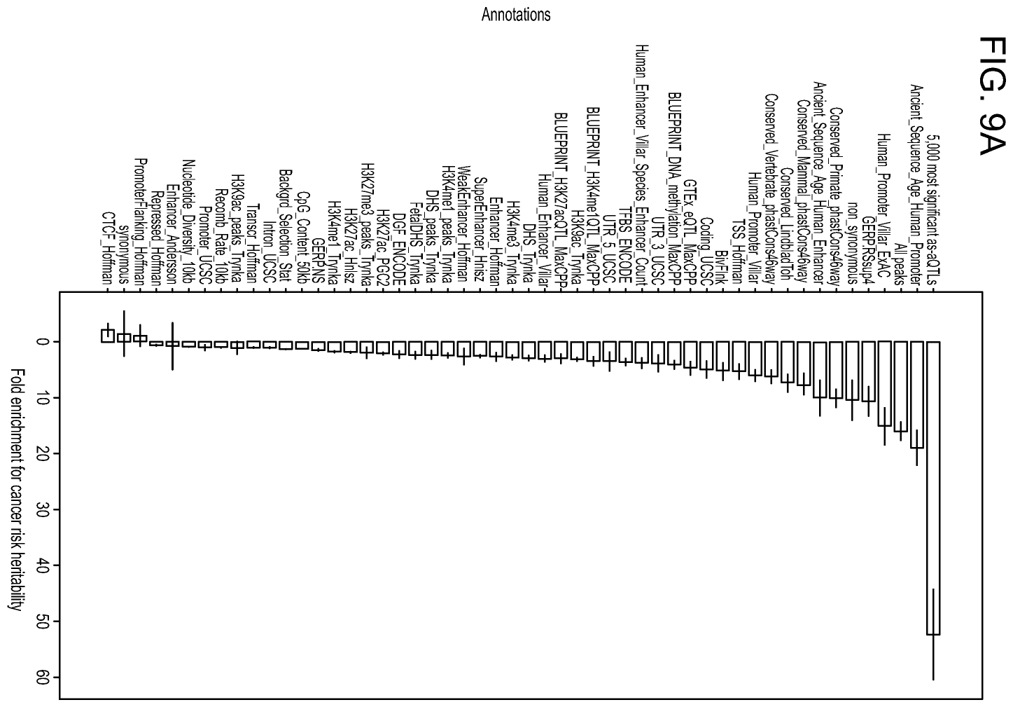

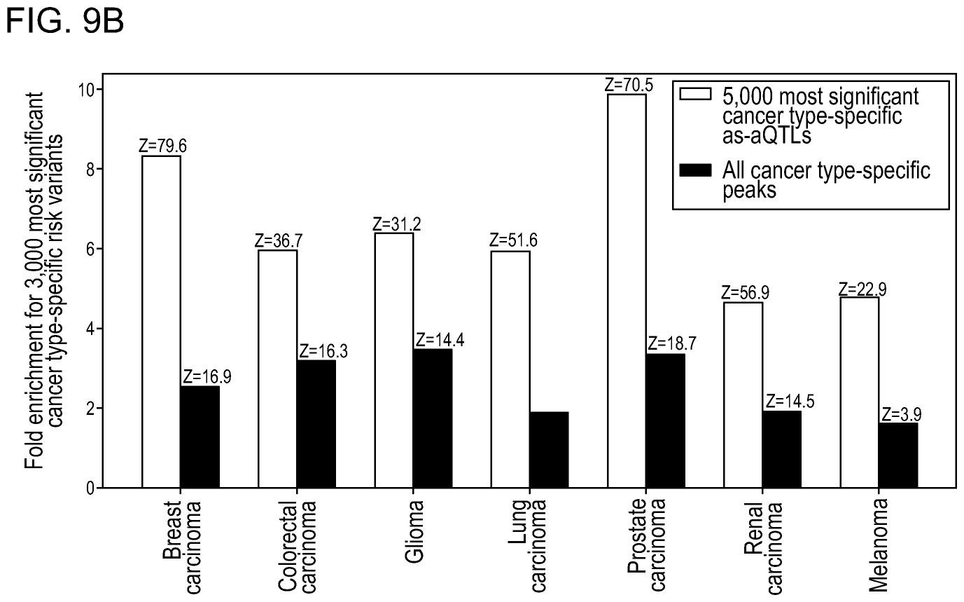

Simplified heritability enrichment analysis. While LDSC estimates polygenic heritability accounting for background annotations, an additional enrichment analysis was conducted for the top GWAS associations. The 5,000 most significant cancer-type specific as-aQTLs were intersected with 3,000 most significant GWAS risk variants (˜ 0.3% of variants from each summary statistic) and calculated the enrichment relative to randomly sampled genomic regions for each cancer type. Significant enrichment of cancer risk associations were found at all cancer type-specific as-aQTLs, which was also considerably higher compared to the enrichment at all peaks (FIG. 9B). Of note, only for breast cancer GWAS summary statistics did the set of 3,000 most significant variants include only genome-wide significant variants. For all other cancer types, the majority of variants were below the genome-wide significance threshold.

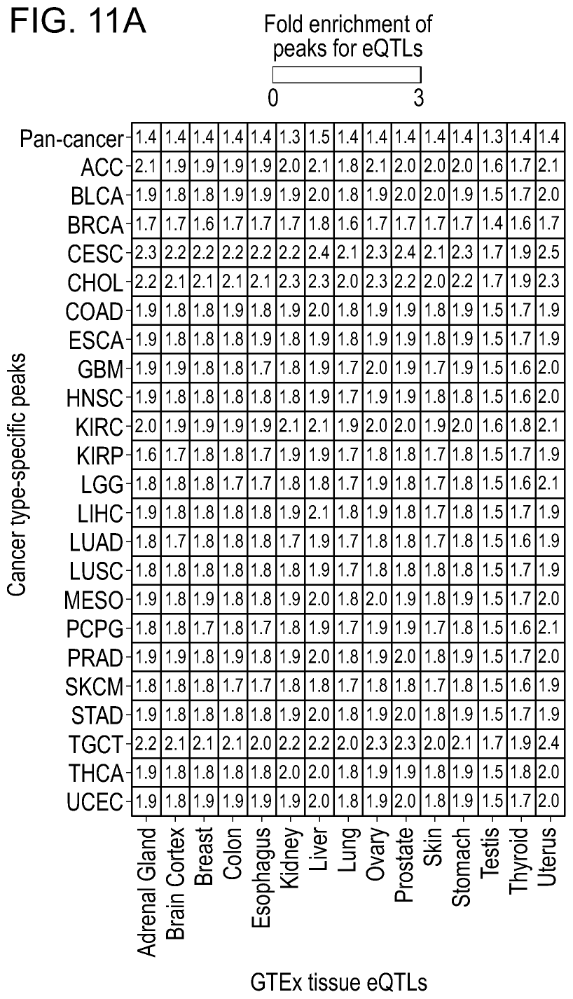

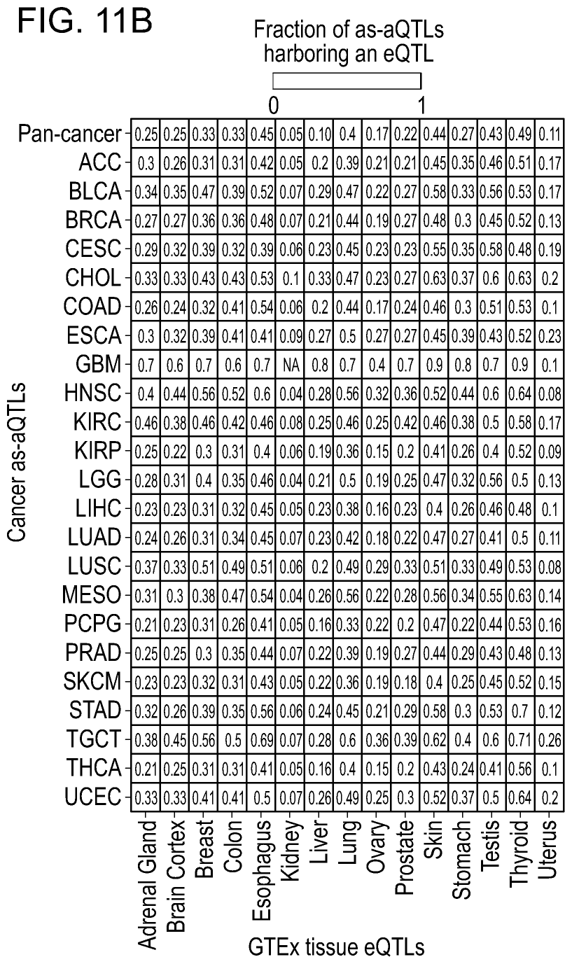

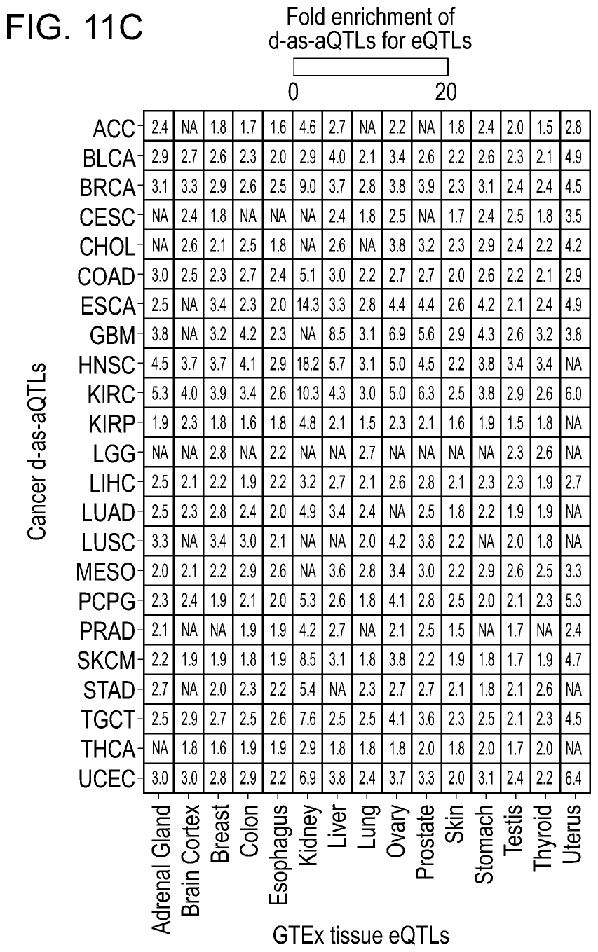

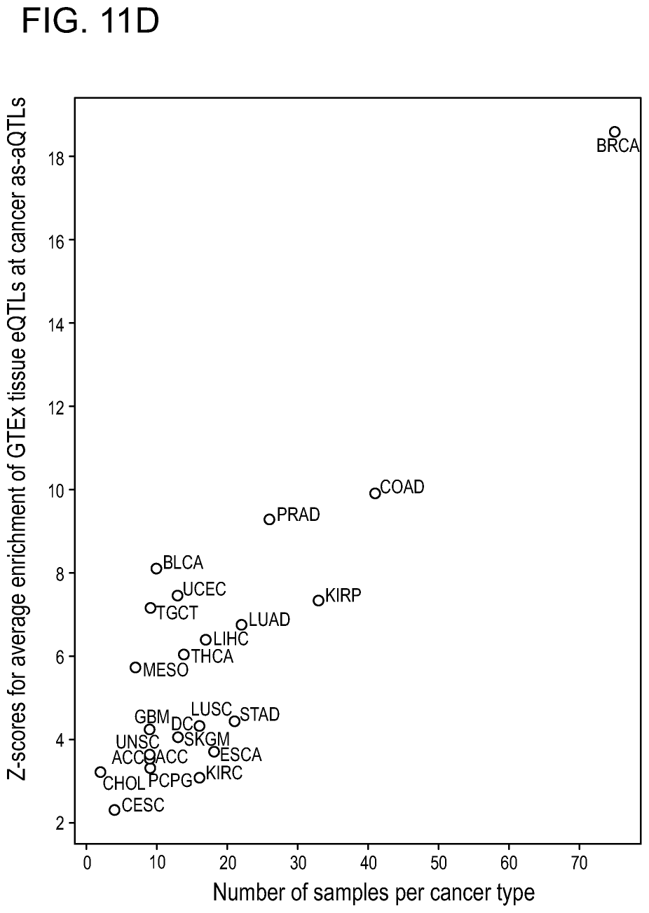

Additional eQTL enrichment analyses. The as-aQTL enrichments relative to all peaks as background were higher than enrichments at all peaks relative to random genomic regions as background (average enrichment of 1.8±0.2; FIG. 11A), showcasing the substantial refinement of candidate causal regulatory variants through as-aQTL analysis. Notably, for many pairs, more than half of as-aQTLs overlapped one or more eQTL, showing qualitatively that the majority of as-aQTLs likely lead to downstream effects on transcription (FIG. 11B). Finally, eQTL enrichment at d-as-aQTLs relative to all peaks produced a slightly larger average enrichment (3.0±1.6) (FIG. 11C) compared to the eQTL enrichment at as-aQTLs, though the difference was not significant. This enrichment analysis using was repeated TCGA cancer tissue eQTLs (Gong, J. et al., Nucleic Acids Res. 46, D971-D976 (2018)). For most matching sample pairs, it was found that as-aQTLs are modestly more enriched for TCGA cancer eQTLs (median 3.15×) than GTEx normal eQTLs (median 2.25×) (Table 4) which is in line with previous findings showing that most eQTLs observed in cancers are consistent with effects in the normal tissues (Geeleher, P. et al., Genome Biol. 19, 130 (2018)).

Transcription factor motif analysis. HOMER (Heinz, S. et al., Mol. Cell 38, 576-589 (2010)) was used to discover TF motifs that are enriched at pan-cancer as-aQTLs (500 bp peaks). To identify the effects of as-aQTL genetic variants on TF motifs, 5406 pan-cancer as-aQTLs were chosen and filtered out as-aQTLs with more than one variant per as-aQTLs which left a set of 2868 as-aQTLs each with a single variant within the 500 bp peak. This was done to reduce noise since, in most cases, it was assumed that only one variant per as-aQTL can be causal in regards to the imbalance. For each as-aQTL genetic variant, a 49 bp sequence window was extracted with the variant at the center. For each 49 bp sequence, two versions, one version with the allele at the center associated with a higher allelic fraction, and a second version with the allele at the center associated with a lower allelic fraction were generated. Both sets of 2,868 sequences were scored using the HOMER motif discovery algorithm for 440 known TF motifs in the HOMER database. 478/2868 as-aQTL variants that caused an absolute change in motif score that was <3 were filtered out to exclude ambiguous motif matching. For the remaining 2,390 variants, two average motif scores for each discovered motif were then calculated; one for the set of sequences with a higher allelic fraction and the other one for the matching set of sequences with a lower allelic fraction. Finally, a paired t-test between each pair of motif score sets was performed and Pearson correlation coefficients were calculated between motif scores and allelic factions. For each variant in an as-aQTL its distance to peak center was also plotted and similarly for variants within balanced peaks.

Integration with SNP-SELEX and SuRE measurements. The results of the imbalance analysis were validated using previously published experimental data. To demonstrate that allelic imbalance is linked to differential TF binding, 2,660,658 SNP-TF pairs from the SNP-SELEX dataset (Yan, J. et al., Nature (2021)) (merged data from original and novel batch) were intersected with 10,005 variants from 5,406 pan-cancer as-aQTLs and Pearson correlation coefficients between as-aQTL allelic fractions and SNP-SELEX SNP-TF pair Preferential Binding Score (PBS) values were calculated at multiple PBS value thresholds. Furthermore, to show that allelic imbalance is associated with differential gene expression, allelic fractions at pan-cancer as-aQTLs variants were correlated with SuRE measurements (van Arensbergen, J. et al., Nat. Biotechnol. 35, 145-153 (2017)) by intersecting 5,919,293 SuRE SNPs measured in HepG2 cells with 10,005 genetic variants from 5,406 pan-cancer as-aQTLs and calculating the Pearson correlations at multiple SuRE SNP p-value thresholds.

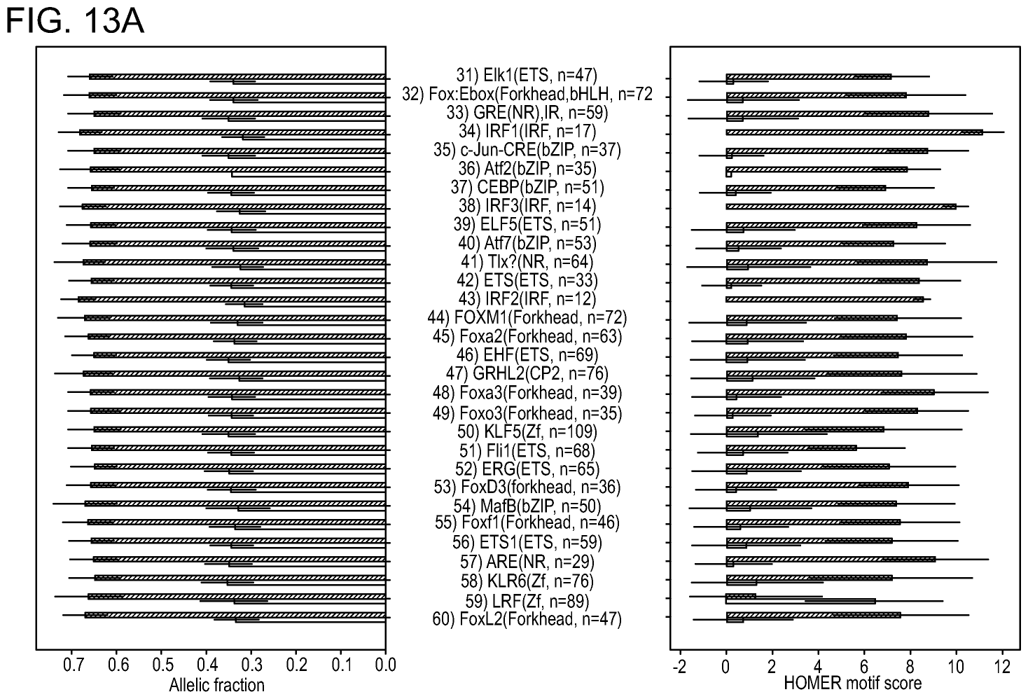

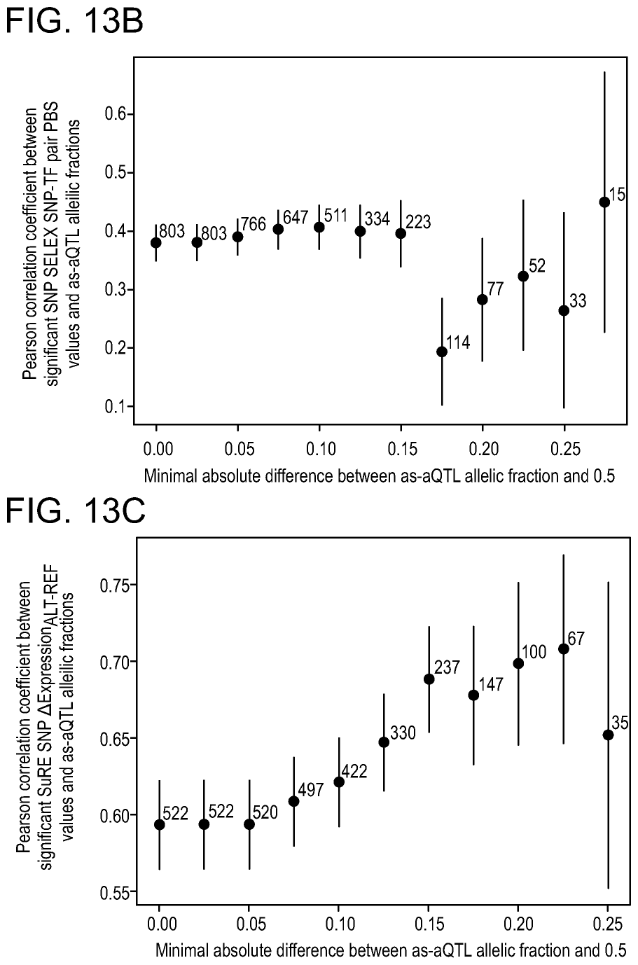

Additional SNP-SELEX and SuRE data analyses. The correlation between significant SNP-SELEX SNP-TF pair PBS values and as-aQTL allelic fractions was not significantly affected when the absolute allelic fraction of the as-aQTLs was instead increased, suggesting that the low correlation at small PBS values was not due to poorly estimated as-aQTLs (FIG. 13B). Equivalent analysis was conducted for SuRE SNPs and yielded similar results. The correlation between allelic fractions and significant SuRE SNP ΔExpression_{ALT-REF} values was not significantly affected by varying as-aQTL allelic fraction thresholds (FIG. 13C). Both analyses were conducted using only significant SNP-SELEX SNP-TF pairs (p<0.01, defined in the original publication. See, Yan, J. et al., Nature (2021)) and SuRE SNPs (p<0.00173121, as defined in the original publication. See, van Arensbergen, J. et al., Nat. Biotechnol. 35, 145-153 (2017)).

Generation of weights for RWAS. Six predictive models were implemented to calculate model weights for the 561,209 pan-cancer peaks. The model types included 3 LASSO penalized regression model types for predicting the accessibility phenotype from all SNPs in the locus (“lasso”) and 3 model types that use the single best SNP from a locus (“top1”). 2 model types used SNP dosages as the predictors and total accessibility activity as the outcome (lasso.total, top1. total), 2 model types that used haplotype differences as the predictors and the allelic ratio at heterozygous individuals as the outcome (lasso.allelic, top1. allelic), and 2 model types that used both the genotype and allele-specific signals together under the assumption that genetic signals were shared (lasso.combined, top1. combined).

Weights for model types that use total activity (topl.total and lasso.total) were calculated from the model y˜X, where y is the normalized ATAC-seq insertion counts after regressing out the covariate matrix of cancer type indicators, and X is a matrix of genotype dosages for all SNPs in the locus. Weights for model types that use allelic activity (top1. allelic and lasso. allelic) were calculated from the model w˜H, where w is the log of the allelic ratio of read counts from the maternal/paternal haplotypes (H1/H0), and H is the matrix of haplotype differences for all SNPs in the locus (defined as the element-wise difference of the H1-HO haplotype matrices). Individuals that did not have a heterozygous, read-carrying variant had an undefined w and were excluded. This formalization of the allelic model is supported by others in the context of statistical fine-mapping (Wang, A. T. et al., Am. J. Hum. Genet. 106, 170-187 (2020)). Both the read count quantification and the haplotype phasing were identical to the main allelic imbalance analysis.

The calculation of weights for top1. combined and lasso. combined model types used both allelic ratios as well as the normalized ATAC-seq insertion counts. Specifically, under the assumption that the two signals have a comparable contribution to the predictive model, y, X, w, and H were centered and scaled and weights calculated from the model [y, w]˜[X, H] where the [ ] operator corresponds to a row-wise concatenation of the two vectors/matrices. A similar combined model has recently been proposed for the analysis of gene expression (Liang, Y. et al., Nat. Commun. 12, 1424 (2021)). Importantly, top1. total and lasso.total model types were trained using a significantly larger set of ATAC-Seq peaks because it was possible to include peaks without variants in the model training (for which measurement of imbalance was not possible). Similarly, to allelic imbalance analysis, a segment mean cutoff at 0.6 was set which excluded individuals with high-CNV genomic regions from model training. The pan-cancer peak set, pan-cancer normalized ATAC-seq insertion counts, and an indicator covariate was used for each cancer type. Penalized regression was carried out using standard LASSO training implemented by the glmnet library (Friedman, J. et al., J. Stat. Softw. 33, 1-22 (2010)), with penalties estimated by internal cross-validation. Model accuracy was then evaluated by an external round of five-fold cross-validation (with the lasso penalty re-learned within each fold to avoid over-fitting to this parameter) (Zhou, D. et al., Nat. Genet. 52, 1239-1246 (2020)). Model training was implemented in the open-source stratAS software and is compatible with RWAS analysis using the FUSION software (Gusev, A. et al., Nat. Genet. 48, 245-252 (2016)).

Conducting RWAS and TWAS. RWAS for 6 model types was conducted using the calculated weights and published summary statistics for eight cancer types (breast cancer (Zhang, H. et al., Nat. Genet. 52, 572-581 (2020)), colorectal cancer (Huyghe, J. R. et al., Nat. Genet. 51, 76-87 (2019)), prostate cancer (Schumacher, F. R. et al., Nat. Genet. 50, 928-936 (2018)), lung cancer (Mckay, J. D. et al., Nat. Genet. 49, 1126-1132 (2017)), kidney cancer (Scelo, G. et al., Nat. Commun. 8, 15724 (2017)), glioma (Melin, B. S. et al., Nat. Genet. 49, 789-794 (2017)), and melanoma (Loh, P. R. et al., Nat. Genet. 50, 906-908 (2018))). In brief, RWAS/TWAS estimates the association between the genetic predictor of accessibility/expression (respectively) and the disease using GWAS summary data and reference panel LD (Gusev, A. et al., Nat. Genet. 48, 245-252 (2016)). Given a vector of RWAS SNP weights, α, trained from each of the models, GWAS test statistics Z, and a matrix of SNP correlations R, the RWAS statistic is computed as Z_{RWAS}=αZ/(αRα′)^{1/2}. The R matrix was computed from a reference panel of European 1000 Genomes samples (1000 Genomes Project Consortium et al., Nature 526, 68-74 (2015)). It should be noted that for the RWAS/TWAS statistics to be properly calibrated, the LD reference must match the target GWAS data, whereas the training ATAC/RNA data is only providing the relative weighting of the SNPs and may not match without introducing bias. TWAS was conducted similarly using pre-computed, published weights with top1. total and lasso.total model types (Mancuso, N. et al., Nat. Commun. 9, 4079 (2018)). Models were considered with cross-validation p-values<0.05 computed for allelic activity or total activity to be “predictive”. Peaks with multiple models were reduced to the single model with the most significant cross-validation p-value. Models were considered RWAS/TWAS significant at p-value<0.05 after Bonferroni correction. The FUSION script FUSION.assoc_test.R was used to carry out RWAS/TWAS analyses.

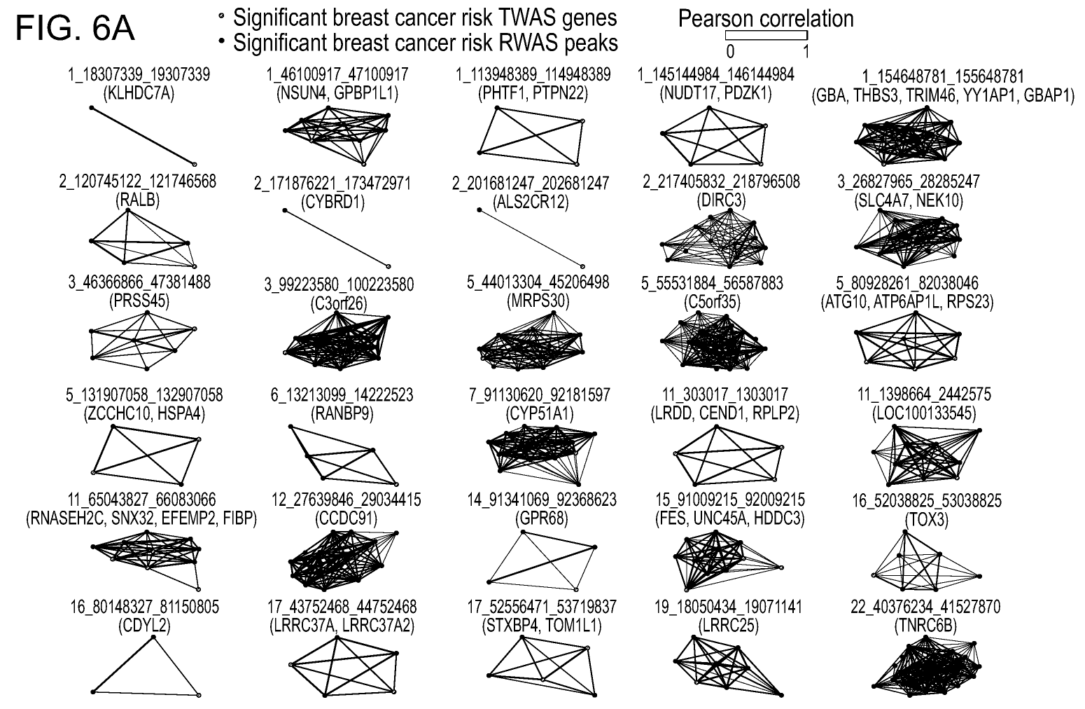

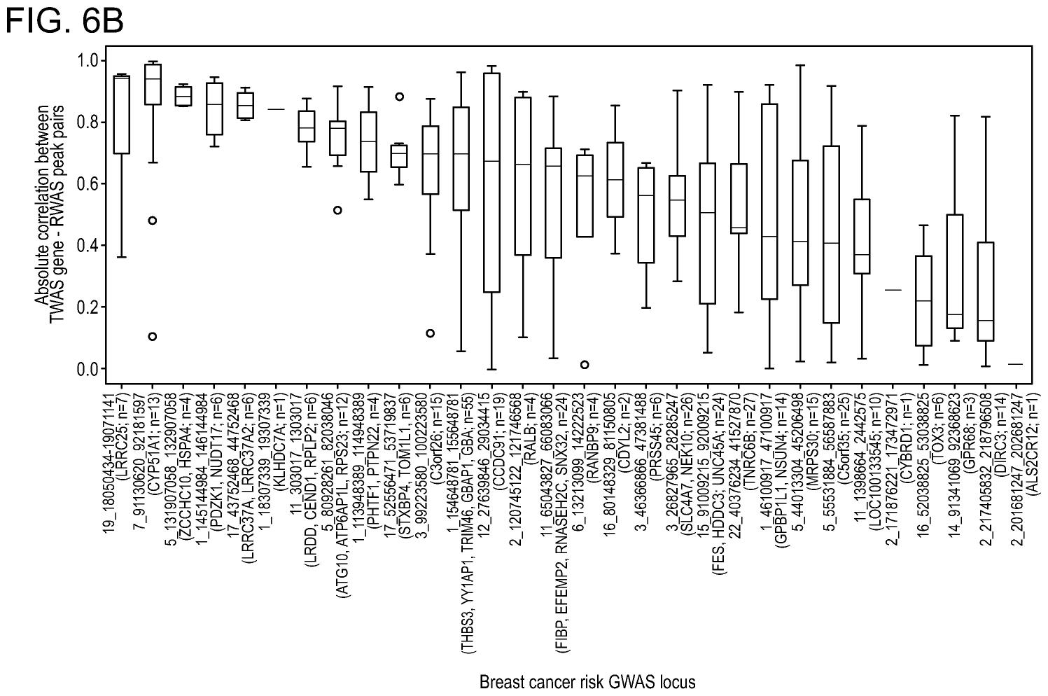

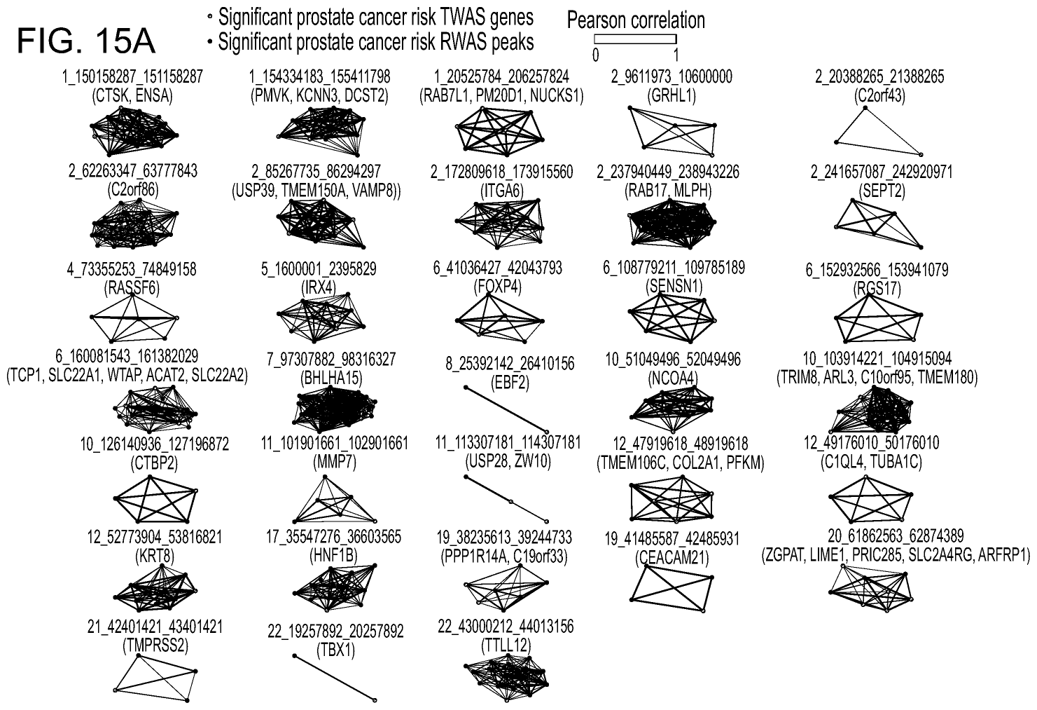

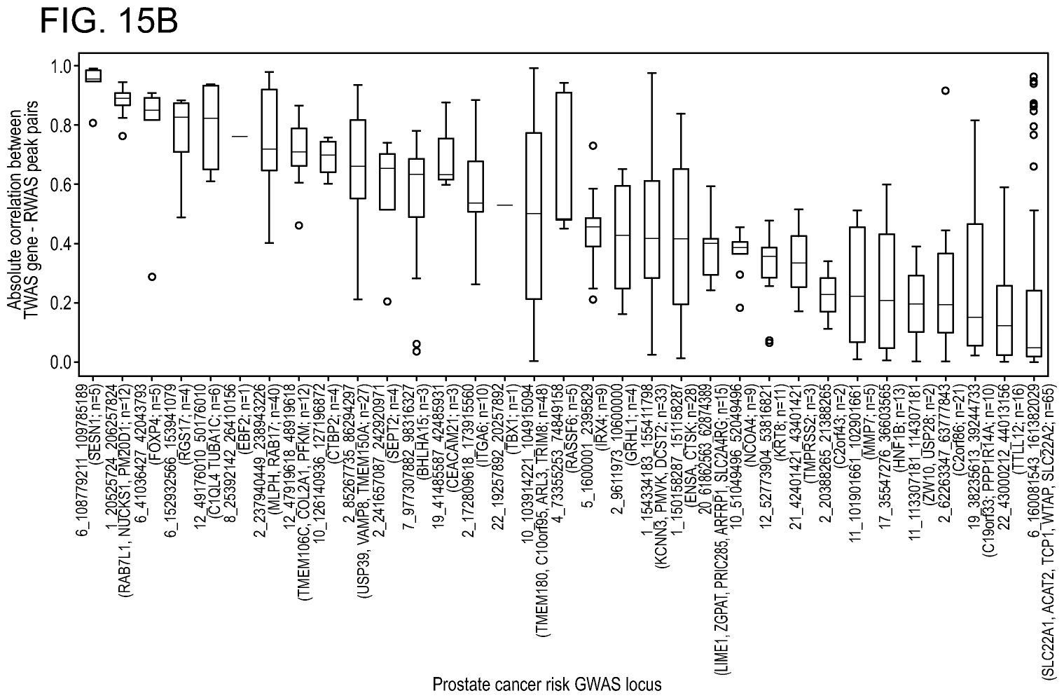

Correlation analyses for RWAS, TWAS, and CWAS. TWAS and RWAS analyses were integrated by imputing peak activity/gene expression for pairs of heritable models (cross-validation p-value <0.05; choosing the most predictive model for each feature) and calculating the absolute Pearson correlation between models that were significantly associated with cancer risk after Bonferroni correction. In FIG. 6A and FIG. 15A, the models are shown as nodes and the correlation as edges between them. Significant CWAS peaks as identified as known in the art and were correlated with RWAS peaks in the same way. See, Meyer, K. B. et al., PLOS Biol. 6, e108 (2008). The FUSION script compare_models.R was used to carry out RWAS/TWAS analyses.

Conditional RWAS analysis. Peaks significantly associated with cancer risk in RWAS were used for conditional RWAS analysis (Gusev, A. et al., Nat. Genet. 50, 538-548 (2018); Yang, J. et al., Nat. Genet. 44, 369-75, S1-3 (2012)). GWAS signals from summary statistics of 7 cancers were independently conditioned on every significant association in every model type. The model type that best explained the GWAS signals was retained for every conditional analysis. The FUSION script FUSION.post_process.R was usedto carry out the conditional analyses.

Combining imbalance analysis, motif discovery, and RWAS. 7 sets of imbalanced genomic regions identified in samples mostly closely matching the 7 available GWAS summary statistics were created by merging cancer type-specific imbalanced, cancer type-specific differentially imbalanced, and pan-cancer imbalanced peaks for BRCA, PRAD, SKCM, COAD, LUAD+LUSC, KIRC+KIRP, and LGG+GBM samples. Duplicate regions were removed by retaining the regions with the statistically most significant imbalance. Transcription factor motif discovery with HOMER was performed as described herein. For each cancer type, the resulting set of variants associated with allelic imbalance and TF motif disruption was intersected with the set of peaks identified in conditional RWAS analysis. Finally, the nearest gene and nearest COSMIC gene (Baca, S. C. et al., bioRxiv 2021.05.10.443466 (2021)) were identified for each variant.

### Example 2: as-aQTLs are Prevalent Across Cancer Types

It has been observed that cancer risk variants discovered in GWAS are enriched at quantitative trait loci for gene expression (eQTLs), providing a potential path towards understanding their mechanisms (Fachal, L. et al., Nat. Genet. 52, 56-73 (2020); Gusev, A. et al., Nat. Genet. 51, 815-823 (2019); Wu, L. et al., Nat. Genet. 50, 968-978 (2018); Mancuso, N. et al., Nat. Commun. 9, 4079 (2018); Hormozdiari, F. et al., Nat. Genet. 50, 1041-1047 (2018)). eQTLs are themselves enriched for regions with epigenetic activity (Liu, X. et al., Am. J. Hum. Genet. 100, 605-616 (2017); Degner, J. F. et al., Nature 482, 390-394 (2012); Gaffney, D. J. et al., Genome Biol. 13, R7 (2012)) and transcription factor (TF) binding motifs, and likely mediated by TF binding (Degner, J. F. et al., Nature 482, 390-394 (2012); Battle, A. & Montgomery, S. B. Hum. Genet. 133, 727-735 (2014); Brown, A. A. et al., Nat. Genet. 49, 1747-1751 (2017)). However, relatively few eQTL studies of cancers have been conducted to date (Gong, J. et al., 46, D971-D976 (2018); Geeleher, P. et al., Genome Biol. 19, 130 (2018); Li, Q. et al., Hum. Mol. Genet. 23, 5294-5302 (2014)) and “steady-state” cis-eQTLs from healthy tissues display low tissue specificity (Liu, X. et al., Am. J. Hum. Genet. 100, 605-616(2017); Gamazon, E. R. et al., Nat. Genet. 50, 956-967 (2018); Mu, Z. et al., Genome Biol. 22, 122 (2021)) and limited colocalization with disease (Yao, D. W. et al., Nat. Genet. 52, 626-633 (2020); Umans, B. D. et al., Trends Genet. 37, 109-124 (2021); Chun, S. et al., Nat. Genet. 49, 600-605 (2017)), which hinders their use for understanding context-specific disease mechanisms. Thus, other types of QTLs may broaden our understanding of non-coding cancer mechanisms.

In addition to eQTLs, QTLs influencing other molecular phenotypes have been identified using various technologies that measure epigenetic activity (Degner, J. F. et al., Nature 482, 390-394 (2012); Waszak, S. M. et al., Cell 162, 1039-1050 (2015); Grubert, F. et al., Cell 162, 1051-1065 (2015); McVicker, G. et al., Science 342, 747-749 (2013); Gate, R. E. et al., Nat. Genet. 50, 1140-1150 (2018)). For example, Assay for Transposase-Accessible Chromatin using sequencing (ATAC-Seq) allows identification of accessible genomic regions which are often associated with transcription factor binding motifs (Gate, R. E. et al., Nat. Genet. 50, 1140-1150 (2018); Liang, D. et al., Nat. Neurosci. (2021)). The effect of a genetic variant on accessibility can be observed both as an association with total activity, and as a difference in activity/binding between two alleles of a heterozygous variant, referred to as allelic imbalance (Pickrell, J. K. et al., Nature 464, 768-772 (2010); Yan, H. et al., Science 297, 1143 (2002); Battle, A. et al., Genome Res. 24, 14-24 (2014); Castel, S. E. et al., Genome Biol. 21, 234 (2020)). The intra-sample nature of allelic imbalance mitigates the effects of technical noise and non-technical confounding (i.e., population stratification) further enhancing its power to identify causal variants (Wang, A. T. et al., Am. J. Hum. Genet. 106, 170-187 (2020); Liang, Y. et al., Nat. Commun. 12, 1424 (2021); Gutierrez-Arcelus, M. et al., Nat. Genet. 52, 247-253 (2020)). Previously, allelic imbalance analysis in cancer samples has been limited to the study of allelic copy number alterations and was not used to study germline regulatory effects.

Here, allelic imbalance was leveraged to identify germline cis-regulatory variants influencing chromatin accessibility across cancers while accounting for cancer-sample specific biases (FIG. 1A-FIG. 1B) (Gusev, A. et al., bioRxiv 631150 (2019)). Allelic imbalance in 406 largely European-ancestry (FIG. 8A) TCGA ATAC-Seq cancer quantified from 23 cancer types (Corces, M. R. et al., Science 362, (2018)) at a total of 649,851 variants inside 338,705 accessible 500 bp peaks (Table 1) were quantified.

TABLE 1
Tumor samples and the results of the imbalance analysis

| | | | | | | as- | | d-as- |
|---|---|---|---|---|---|---|---|---|
| | | | | | as- | aQTL | d-as- | aQTL |
| | | | | # tested | aQTL | peaks | aQTL | peaks |
| | | | # tested | d-as- | variants | at | variants | at |
| | | # of | as-aQTL | aQTL | at 10% | 10% | at 10% | 10% |
| Cancer Type | Acronym | Samples | variants | variants | FDR | FDR | FDR | FDR |
| Adrenocortical | ACC | 9 | 63604 | 63512 | 169 | 80 | 83 | 47 |
| Carcinoma | | | | | | | | |
| Bladder Urothelial | BLCA | 10 | 225811 | 225537 | 354 | 178 | 142 | 80 |
| Carcinoma | | | | | | | | |
| Breast Invasive | BRCA | 75 | 552892 | 549354 | 2580 | 1383 | 281 | 155 |
| Carcinoma | | | | | | | | |
| Cervical Squamous | CESC | 4 | 81946 | 81869 | 62 | 31 | 40 | 23 |
| Cell Carcinoma | | | | | | | | |
| Cholangiocarcinoma | CHOL | 2 | 97499 | 97390 | 75 | 30 | 21 | 15 |
| Colon | COAD | 41 | 384306 | 383337 | 958 | 470 | 243 | 132 |
| Adenocarcinoma | | | | | | | | |
| Esophageal | ESCA | 18 | 321286 | 320658 | 80 | 44 | 26 | 16 |
| Carcinoma | | | | | | | | |
| Glioblastoma | GBM | 9 | 95435 | 95321 | 16 | 10 | 15 | 8 |
| Multiforme | | | | | | | | |
| Head and Neck | HNSC | 9 | 314792 | 314242 | 43 | 25 | 22 | 15 |
| Squamous Cell | | | | | | | | |
| Carcinoma | | | | | | | | |
| Kidney Renal Clear | KIRC | 16 | 155116 | 154872 | 44 | 24 | 22 | 16 |
| Cell Carcinoma | | | | | | | | |
| Kidney Renal | KIRP | 33 | 412908 | 411925 | 1217 | 607 | 210 | 110 |
| Papillary Cell | | | | | | | | |
| Carcinoma | | | | | | | | |
| Low Grade Glioma | LGG | 13 | 353176 | 352349 | 116 | 68 | 13 | 9 |
| Liver Hepatocellular | LIHC | 17 | 282694 | 282244 | 575 | 311 | 210 | 118 |
| Carcinoma | | | | | | | | |
| Lung | LUAD | 22 | 415265 | 414275 | 531 | 240 | 51 | 31 |
| Adenocarcinoma | | | | | | | | |
| Lung Squamous Cell | LUSC | 16 | 320083 | 319378 | 91 | 49 | 45 | 23 |
| Carcinoma | | | | | | | | |
| Mesothelioma | MESO | 7 | 297074 | 296585 | 192 | 94 | 50 | 30 |
| Pheochromocytoma | PCPG | 9 | 259274 | 258860 | 147 | 81 | 61 | 37 |
| and Paraganglioma | | | | | | | | |
| Prostate | PRAD | 26 | 515181 | 512893 | 1010 | 512 | 85 | 48 |
| Adenocarcinoma | | | | | | | | |
| Skin Cutaneous | SKCM | 13 | 243309 | 242921 | 230 | 109 | 91 | 51 |
| Melanoma | | | | | | | | |
| Stomach | STAD | 21 | 311506 | 310963 | 121 | 66 | 53 | 34 |
| Adenocarcinoma | | | | | | | | |
| Testicular Germ Cell | TGCT | 9 | 213022 | 212678 | 167 | 80 | 80 | 46 |
| Tumors | | | | | | | | |
| Thyroid Carcinoma | THCA | 14 | 209243 | 209006 | 707 | 325 | 175 | 88 |
| Uterine Corpus | UCEC | 13 | 219402 | 219033 | 300 | 151 | 94 | 59 |
| Endometrial | | | | | | | | |
| Carcinoma | | | | | | | | |
| All cancer types | PANCAN | 406 | 649851 | NA | 10005 | 5406 | NA | NA |
| Testicular Germ Cell | TGCT | 9 | 213022 | 212678 | 167 | 80 | 80 | 46 |
| Tumors | | | | | | | | |

A pan-cancer analysis across all 406 cancer samples identified 5,406 as-aQTLs at 10% false discovery rate (FDR), demonstrating that as-aQTLs are pervasive across cancer types. Downsampling showed that the population-scale approach maximized statistical power with no sign of saturation (FIG. 8B). The analysis described herein was robust to alternative peak definitions (FIG. 8C) and conservatively controlled for extreme somatic copy number alterations (FIG. 8D). Within each cancer type, the number of significant peaks (at 10% FDR) varied from 10 (in 9 glioblastoma samples) to 1,383 (in 75 breast cancer samples) and was driven largely by sample size (FIG. 1C, Table 2A-Table 2W), with a union of 7,262 as-aQTLs across all cancers. The as-aQTLs in Table 2A-Table 2W represent potential biomarkers representing allelic imbalanced variants discovered in specific cancers. Additionally, between 8 (in 9 glioblastoma samples) and 155 (in 75 breast cancer samples) were identified as “differentially” imbalanced regions (differential allelic-specific accessibility QTLs; d-as-aQTLs) for each cancer type by testing for a difference in the allelic fraction relative to all other cancers, for a union of 1,096 d-as-aQTLs across all cancers (FIG. 1C, Table 3A-Table 3W). Similarly, the d-as-aQTLs in Table 3A-Table 3W also represent potential biomarkers. As a representative example, individual-level allelic imbalance was shown for a breast-specific as-aQTL/d-as-aQTL at variant rs7422038 (FIG. 1D-FIG. 1E). These results demonstrate that these methods can robustly discover as-aQTLs in cancer samples.
Table 2A-Table 2W: as-aQTL variants discovered at 10% FDR in 23 cancer types and pan-cancer analysis
Table 2A-Table 2W are located within the Appendix to the Specification at pages 1 to 191.
Table 3A-Table 3W: d-as-aQTL variants discovered at 10% FDR in 23 cancer types Table 3A-Table 3W are located within the Appendix to the Specification at pages 192 to 214.

### Example 3: Cancer risk heritability is highly enriched at cancer as-aQTLs

The extent to which cancer as-aQTLs play a role in the mechanisms of cancer risk were investigated by carrying out heritability enrichment analysis using seven publicly available cancer risk GWAS (breast cancer (Zhang, H. et al., Nat. Genet. 52, 572-581 (2020)), colorectal cancer (Huyghe, J. R. et al., Nat. Genet. 51, 76-87 (2019)), prostate cancer (Schumacher, F. R. et al., Nat. Genet. 50, 928-936 (2018)), lung cancer (Mckay, J. D. et al., Nat. Genet. 49, 1126-1132 (2017)), kidney cancer (Scelo, G. et al., Nat. Commun. 8, 15724 (2017)), glioma (Melin, B. S. et al., Nat. Genet. 49, 789-794 (2017)), and melanoma (Loh, P. R. et al., Nat. Genet. 50, 906-908 (2018))). Stratified LD score regression (LDSC) was applied to estimate heritability enrichment in the context of a baseline model (Gazal, S. et al., Nat. Genet. 51, 1202-1204 (2019)) that accounts for background enrichment near generic regulatory elements. For three cancer types with the most as-aQTLs (breast cancer, prostate cancer, and renal cancer), as-aQTLs exhibited significant heritability enrichment compared to all peaks, and the enrichment increased when restricting to more significant as-aQTLs, further supporting the hypothesis that allelic imbalance drives this enrichment (FIG. 2A-FIG. 2C). For example, the 1,000 most significant prostate cancer as-aQTLs showed a 145.6±35.7 fold enrichment for prostate cancer risk heritability, compared to a 26.6±4.0 fold enrichment for all prostate cancer accessible peaks. Furthermore, a meta-analysis across all seven cancer types showed significant enrichment of cancer risk heritability at corresponding cancer as-aQTLs (52.4±8.1) compared to corresponding accessible regions (16.0±1.7), previously fine-mapped GTEx eQTLs (5.0±1.2; Methods) (Hormozdiari, F. et al., Nat. Genet. 50, 1041-1047 (2018)), or coding regions (4.7±1.6), and all other available annotations (FIG. 2D and FIG. 9A). It was observed that significant enrichment within each cancer type when quantified using a simpler strategy based on top GWAS associations that does not account for background annotations (FIG. 9B). Finally, cancer type-specific as-aQTL did not show heritability enrichment for non-cancer traits (FIG. 10A-FIG. 10C) and displayed a significantly higher heritability enrichment for matching cancer than pan-cancer as-aQTLs (FIG. 10D-FIG. 10F). These results show that as-aQTLs are strongly and specifically enriched for cancer risk heritability in every cancer type that was available for analysis.

### Example 4: Cancer as-aQTLs are Associated with Transcriptional Regulation

Next, it was investigated whether as-aQTLs may have downstream effects on the transcription of nearby genes. First, it was found that as-aQTLs were 1.4× enriched in promoters (+/−2kb of a transcription start site) relative to all peaks (FIG. 2E). Then, the enrichment of eQTLs at as-aQTLs was investigated using significant eQTLs identified in the GTEx (v8) (GTEx Consortium. Science 369, 1318-1330 (2020)) analysis of 15 tissue types that most closely matched the 23 cancer types in our ATAC-Seq dataset. Cancer as-aQTLs were significantly enriched for GTEx eQTLs for 347 out of 360 GTEx tissue-cancer pairs (compared to randomly sampled accessible peaks), with an average enrichment of 2.5±1.0 (FIG. 2F). Notably, enrichment of eQTLs at matching cancer-tissue pairs was not significantly higher than at non-matching pairs (FIG. 2G), consistent with previous observations that cis-eQTLs are highly shared across tissues (Liu, X. et al., Am. J. Hum. Genet. 100, 605-616 (2017); GTEx Consortium. Science 369, 1318-1330 (2020); GTEx Consortium et al., Nature 550, 204-213 (2017)) and cell-types (Mu, Z. et al., Genome Biol. 22, 122 (2021)). These findings confirm that aQTLs are enriched for downstream effects on gene expression in both cancer and healthy tissues; however, the substantially higher enrichment of cancer risk heritability at as-aQTLs over eQTLs indicates the former captures additional disease mechanisms not observed in the latter. For additional eQTL enrichments, see FIG. 11A-FIG. 11D, and Table 4.

TABLE 4
as-aQTL enrichments for GTEx normal eQTLs and TCGA tumor eQTLs

| | Enrichment for | Enrichment for |
|---|---|---|
| | matching TCGA | matching healthy |
| Tumor as-aQTLs | tumor tissue eQTLs* | GTEx tissue eQTLs* |
| Breast Invasive Carcinoma (BRCA) | 3.13628 (16.2919) | 2.24359 (18.5153) |
| Prostate Adenocarcinoma (PRAD) | 2.37369 (11.5188) | 2.4951 (11.4505) |
| Kidney Renal Clear Cell Carcinoma (KIRP) | 3.22767 (9.58636) | 4.31809 (9.01795) |
| Thyroid Carcinoma (THCA) | 2.12149 (8.18804) | 1.80268 (9.54072) |
| Adrenocortical Carcinoma (ACC) | 19.9998 (6.30155) | 2.25562 (4.72031) |
| Uterine Corpus Endometrial Carcinoma (UCEC) | 8.74997 (6.29612) | 3.86052 (7.93094) |
| Colon Adenocarcinoma (COAD | 2.6931 (6.2733) | 2.216 (12.6615) |
| Lung Adenocarcinoma (LUAD) | 3.1693 (6.11609) | 1.92158 (8.15444) |
| Lung Squamous Cell Carcinoma (LUSC) | 4.7619 (5.47738) | 2.2242 (4.67392) |
| Low Grade Glioma (LGG) | 3.15683 (5.00724) | 2.29254 (4.24707) |
| Liver Hepatocellular Carcinoma (LIHC) | 2.48758 (4.65394) | 2.48629 (7.46559) |
| Skin Cutaneous Melanoma (SKCM) | 3.99998 (1.39173) | 1.65907 (3.90037) |
| Kidney Renal Clear Cell Carcinoma (KIRC) | 1.86055 (1.2504) | 4.54546 (2.71686) |
| Testicular Germ Cell Tumors (TGCT) | 0.751988 (−0.275317) | 2.17364 (8.03873) |
| Glioblastoma Multiforme (GBM) | No overlap^{‡} | 4.25538 (3.67796) |
| Esophageal Carcinoma (ESCA) | No overlap^{‡} | 1.56389 (2.2961) |
| Stomach Adenocarcinoma (STAD) | No overlap^{‡} | 2.11199 (3.73) |
| Bladder Urothelial Carcinoma (BLCA) | 3.06358 (4.1608) | No matching^{†} |
| Pheochromocytoma and Paraganglioma (PCPG) | 2.94104 (2.00816) | No matching^{†} |
| Mesothelioma (MESO) | 3.33332 (1.17728) | No matching^{†} |
| Head and Neck Squamous Cell Carcinoma (HNSC) | 2.08334 (1.06823) | No matching^{†} |
| Cervical Squamous Cell Carcinoma (CESC) | 1.92308 (0.728664) | No matching^{†} |
| Cholangiocarcinoma (CHOL) | No overlap^{‡} | No matching^{†} |
| Median | 3.15 | 2.25 |

*Z-score
^{†}Matching normal tissue not available
^{‡}No overlap with as-aQTLs

### Example 5: Cancer as-aQTLs interact with Motifs of Oncogenic TFs

The enrichment of as-aQTLs for eQTLs and cancer risk heritability led to the hypothesis that as-aQTLs may harbor binding motifs of oncogenic TFs (i.e., TFs implicated in tumor-specific dysregulation). As hypothesized, a motif enrichment analysis across the 5,406 pan-cancer as-aQTLs (using motifs from the HOMER database) (Heinz, S. et al., Mol. Cell 38, 576-589 (2010)) identified significant enrichment for 132/440 known motifs (q-value<0.05; Table 5A). The 10 most significantly enriched TF motifs belonged to the bZIP motif family. They are bound by TFs (JUN, FOS, FRA2, FOSL2, FRA1, ATF3, BATF, JUNB, BACH2) that form the API transcription complex, which is known to play a role in many cancers (Watt, A. C. et al., Nature Cancer 2, 34-48 (2020); Eferl, R. & Wagner, E. F. Nat. Rev. Cancer 3, 859-868 (2003); Verde, P., Casalino, L., Talotta, F., Yaniv, M. & Weitzman, J. B. Cell Cycle 6, 2633-2639 (2007); Kharman-Biz, A. et al., BMC Cancer 13, 441 (2013)). The next 10 were motifs that are bound by oncogenic TFs that belong to the Forkhead families (FOXA2 (Tang, Y. et al., Cell Res. 21, 316-326 (2011)), FOXA1 (Parolia, A. et al., Nature 571, 413-418 (2019)), and FOXMI (Radhakrishnan, S. K. & Gartel, A. L. Cancer vol. 8 cl; author reply c2(2008))) and ETS (ELF5 (Chakrabarti, R. et al., Nat. Cell Biol. 14, 1212-1222 (2012)), ELK4 (Peng, C. et al., Oncogene 35, 1170-1179 (2016)), ELF1 (Cheng, M. et al., Mol. Ther. Nucleic Acids 23, 418-430 (2021)), ETV1 (Jané-Valbuena, J. et al., Cancer Res. 70, 2075-2084 (2010)), ETV4 (Pellecchia, A. et al., Oncogenesis 1, e20 (2012)), and FLI1 (Miao, B. et al., Int. J. Cancer 147, 189-201 (2020))).

TABLE 5A
TF motifs enriched at pan-cancer as-aQTLs relative to all pan-cancer peaks using
HOMER (Fisher exact test)

| | | | | | # | % of | # | |
|---|---|---|---|---|---|---|---|---|
| | | | | | Target | Targets | Background | |
| | | | | | Sequences | Sequences | Sequences | % |
| | | p- | Log P- | q-value | with | with | with | Back- |
| Rank | Name | value | pvalue | (Benjamini) | Motif | Motif | Motif | ground* |
| 1 | Jun-AP1(bZIP)/K562-cJun-ChIP- | 1.00E−43 | −9.98E+01 | 0 | 821 | 15.19% | 48870.7 | 9.26% |
| | Seq(GSE31477)/Homer | | | | | | | |
| 2 | Fos(bZIP)/TSC-Fos-ChIP- | 1.00E−42 | −9.73E+01 | 0 | 1445 | 26.73% | 100680.6 | 19.08% |
| | Seq(GSE110950)/Homer | | | | | | | |
| 3 | Fra2(bZIP)/Striatum-Fra2-ChIP- | 1.00E−41 | −9.63E+01 | 0 | 1259 | 23.29% | 85032.9 | 16.11% |
| | Seq(GSE43429)/Homer | | | | | | | |
| 4 | Fos12(bZIP)/3T3L1-Fos12-ChIP- | 1.00E−41 | −9.53E+01 | 0 | 992 | 18.35% | 63132.3 | 11.96% |
| | Seq(GSE56872)/Homer | | | | | | | |
| 5 | Fra1(bZIP)/BT549-Fra1-ChIP- | 1.00E−41 | −9.51E+01 | 0 | 1384 | 25.60% | 95882.3 | 18.17% |
| | Seq(GSE46166)/Homer | | | | | | | |
| 6 | Atf3(bZIP)/GBM-ATF3-ChIP- | 1.00E−40 | −9.43E+01 | 0 | 1538 | 28.45% | 109319 | 20.71% |
| | Seq(GSE33912)/Homer | | | | | | | |
| 7 | BATF(bZIP)/Th17-BATF-ChIP- | 1.00E−39 | −9.00E+01 | 0 | 1496 | 27.67% | 106556 | 20.19% |
| | Seq(GSE39756)/Homer | | | | | | | |
| 8 | JunB(bZIP)/DendriticCells-Junb- | 1.00E−39 | −8.99E+01 | 0 | 1354 | 25.05% | 94322.5 | 17.87% |
| | ChIP-Seq(GSE36099)/Homer | | | | | | | |
| 9 | AP-1(bZIP)/ThioMac-PU.1-ChIP- | 1.00E−36 | −8.40E+01 | 0 | 1605 | 29.69% | 117435 | 22.25% |
| | Seq(GSE21512)/Homer | | | | | | | |
| 10 | Bach2(bZIP)/OCILy7-Bach2- | 1.00E−25 | −5.85E+01 | 0 | 562 | 10.40% | 34669.3 | 6.57% |
| | ChIP-Seq(GSE44420)/Homer | | | | | | | |
| 11 | Foxa2(Forkhead)/Liver-Foxa2- | 1.00E−17 | −4.03E+01 | 0 | 1411 | 26.10% | 111764.2 | 21.18% |
| | ChIP-Seq(GSE25694)/Homer | | | | | | | |
| 12 | FOXA1(Forkhead)/MCF7- | 1.00E−15 | −3.54E+01 | 0 | 1696 | 31.37% | 139603.3 | 26.45% |
| | FOXA1-ChIP- | | | | | | | |
| | Seq(GSE26831)/Homer | | | | | | | |
| 13 | FOXM1(Forkhead)/MCF7- | 1.00E−15 | −3.48E+01 | 0 | 1722 | 31.85% | 142226.6 | 26.95% |
| | FOXM1-ChIP- | | | | | | | |
| | Seq(GSE72977)/Homer | | | | | | | |
| 14 | ELF5(ETS)/T47D-ELF5-ChIP- | 1.00E−14 | −3.41E+01 | 0 | 1223 | 22.62% | 96821.8 | 18.35% |
| | Seq(GSE30407)/Homer | | | | | | | |
| 15 | FOXA1(Forkhead)/LNCAP- | 1.00E−14 | −3.38E+01 | 0 | 1928 | 35.66% | 161846.9 | 30.67% |
| | FOXA1-ChIP- | | | | | | | |
| | Seq(GSE27824)/Homer | | | | | | | |
| 16 | Elk4(ETS)/Hela-Elk4-ChIP- | 1.00E−14 | −3.31E+01 | 0 | 910 | 16.83% | 69309 | 13.13% |
| | Seq(GSE31477)/Homer | | | | | | | |
| 17 | ELF1(ETS)/Jurkat-ELF1-ChIP- | 1.00E−14 | −3.24E+01 | 0 | 867 | 16.04% | 65736.9 | 12.46% |
| | Seq(SRA014231)/Homer | | | | | | | |
| 18 | ETV1(ETS)/GIST48-ETV1-ChIP- | 1.00E−14 | −3.24E+01 | 0 | 2041 | 37.75% | 173075.2 | 32.79% |
| | Seq(GSE22441)/Homer | | | | | | | |
| 19 | Fli1(ETS)/CD8-FLI-ChIP- | 1.00E−13 | −3.17E+01 | 0 | 1701 | 31.47% | 141493.5 | 26.81% |
| | Seq(GSE20898)/Homer | | | | | | | |
| 20 | ETV4(ETS)/HepG2-ETV4-ChIP- | 1.00E−13 | −3.09E+01 | 0 | 1714 | 31.71% | 143024.6 | 27.10% |
| | Seq(ENCODE)/Homer | | | | | | | |
| 21 | Elk1(ETS)/Hela-Elk1-ChIP- | 1.00E−13 | −3.02E+01 | 0 | 906 | 16.76% | 69833.7 | 13.23% |
| | Seq(GSE31477)/Homer | | | | | | | |
| 22 | GABPA(ETS)/Jurkat-GABPa- | 1.00E−12 | −2.93E+01 | 0 | 1402 | 25.93% | 114859.7 | 21.76% |
| | ChIP-Seq(GSE17954)/Homer | | | | | | | |
| 23 | Sp1(ZI)/Promoter/Homer | 1.00E−12 | −2.90E+01 | 0 | 585 | 10.82% | 42326.8 | 8.02% |
| 24 | KLF1(Zf)/HUDEP2-KLF1- | 1.00E−12 | −2.80E+01 | 0 | 1638 | 30.30% | 137193.7 | 25.99% |
| | CutnRun(GSE136251)/Homer | | | | | | | |
| 25 | Foxa3(Forkhead)/Liver-Foxa3- | 1.00E−12 | −2.79E+01 | 0 | 700 | 12.95% | 52495.3 | 9.95% |
| | ChIP-Seq(GSE77670)/Homer | | | | | | | |
| 26 | EHF(ETS)/LoVo-EHF-ChIP- | 1.00E−11 | −2.75E+01 | 0 | 1930 | 35.70% | 164771 | 31.22% |
| | Seq(GSE49402)/Homer | | | | | | | |
| 27 | NF1(CTF)/LNCAP-NF1-ChIP- | 1.00E−11 | −2.70E+01 | 0 | 890 | 16.46% | 69443 | 13.16% |
| | Seq(Unpublished)/Homer | | | | | | | |
| 28 | ERG(ETS)/VCaP-ERG-ChIP- | 1.00E−11 | −2.70E+01 | 0 | 2300 | 42.55% | 200145.8 | 37.92% |
| | Seq(GSE14097)/Homer | | | | | | | |
| 29 | Elf4(ETS)/BMDM-Elf4-ChIP- | 1.00E−11 | −2.61E+01 | 0 | 1567 | 28.99% | 131415.9 | 24.90% |
| | Seq(GSE88699)/Homer | | | | | | | |
| 30 | NF1-halfsite(CTF)/LNCaP-NF1- | 1.00E−11 | −2.54E+01 | 0 | 2617 | 48.41% | 231450.1 | 43.85% |
| | ChIP-Seq(Unpublished)/Homer | | | | | | | |
| 31 | Fox:Ebox(Forkhead, bHLH)/Panc | 1.00E−10 | −2.40E+01 | 0 | 1582 | 29.26% | 133742.6 | 25.34% |
| | 1-Foxa2-ChIP- | | | | | | | |
| | Seq(GSE47459)/Homer | | | | | | | |
| 32 | ETS1(ETS)/Jurkat-ETS1-ChIP- | 1.00E−10 | −2.38E+01 | 0 | 1572 | 29.08% | 132907.4 | 25.18% |
| | Seq(GSE17954)/Homer | | | | | | | |
| 33 | FoxL2(Forkhead)/Ovary-FoxL2- | 1.00E−10 | −2.36E+01 | 0 | 1384 | 25.60% | 115567 | 21.90% |
| | ChIP-Seq(GSE60858)/Homer | | | | | | | |
| 34 | ETS(ETS)/Promoter/Homer | 1.00E−10 | −2.31E+01 | 0 | 545 | 10.08% | 40460.3 | 7.67% |
| 35 | CTCF(Zf)/CD4+-CTCF-ChIP- | 1.00E−09 | −2.28E+01 | 0 | 356 | 6.59% | 24583.3 | 4.66% |
| | Seq(Barski_et_al.)/Homer | | | | | | | |
| 36 | Nrf2(bZIP)/Lymphoblast-Nrf2- | 1.00E−09 | −2.24E+01 | 0 | 157 | 2.90% | 8917.5 | 1.69% |
| | ChIP-Seq(GSE37589)/Homer | | | | | | | |
| 37 | GRHL2(CP2)/HBE-GRHL2- | 1.00E−09 | −2.20E+01 | 0 | 655 | 12.12% | 50354.4 | 9.54% |
| | ChIP-Seq(GSE46194)/Homer | | | | | | | |
| 38 | ELF3(ETS)/PDAC-ELF3-ChIP- | 1.00E−09 | −2.19E+01 | 0 | 1175 | 21.74% | 97081.8 | 18.39% |
| | Seq(GSE64557)/Homer | | | | | | | |
| 39 | Etv2(ETS)/ES-ER71-ChIP- | 1.00E−09 | −2.16E+01 | 0 | 1448 | 26.79% | 122421.3 | 23.20% |
| | Seq(GSE59402)/Homer | | | | | | | |
| 40 | KLF5(Zf)/LoVo-KLF5-ChIP- | 1.00E−09 | −2.16E+01 | 0 | 2119 | 39.20% | 185634.5 | 35.17% |
| | Seq(GSE49402)/Homer | | | | | | | |
| 41 | KLF3(Zf)/MEF-Klf3-ChIP- | 1.00E−09 | −2.13E+01 | 0 | 984 | 18.20% | 79930.6 | 15.14% |
| | Seq(GSE44748)/Homer | | | | | | | |
| 42 | EWS:FLI1- | 1.00E−09 | −2.10E+01 | 0 | 908 | 16.80% | 73190.9 | 13.87% |
| | fusion(ETS)/SK_N_MC- | | | | | | | |
| | EWS:FLI1-ChIP- | | | | | | | |
| | Seq(SRA014231)/Homer | | | | | | | |
| 43 | Foxf1(Forkhead)/Lung-Foxf1- | 1.00E−09 | −2.08E+01 | 0 | 1472 | 27.23% | 125015.7 | 23.69% |
| | ChIP-Seq(GSE77951)/Homer | | | | | | | |
| 44 | FOXK1(Forkhead)/HEK293- | 1.00E−08 | −2.06E+01 | 0 | 1584 | 29.30% | 135591.4 | 25.69% |
| | FOXK1-ChIP- | | | | | | | |
| | Scq(GSE51673)/Homer | | | | | | | |
| 45 | Sp2(Zf)/HEK293-Sp2.eGFP- | 1.00E−08 | −2.02E+01 | 0 | 2312 | 42.77% | 204896.4 | 38.82% |
| | ChIP-Seq(Encode)/Homer | | | | | | | |
| 46 | BORIS(Zf)/K562-CTCFL-ChIP- | 1.00E−08 | −1.97E+01 | 0 | 489 | 9.05% | 36619.2 | 6.94% |
| | Seq(GSE32465)/Homer | | | | | | | |
| 47 | NFY(CCAAT)/Promoter/Homer | 1.00E−08 | −1.93E+01 | 0 | 1049 | 19.40% | 86698.7 | 16.43% |
| 48 | Stat3(Stat)/mES-Stat3-ChIP- | 1.00E−08 | −1.93E+01 | 0 | 815 | 15.08% | 65521 | 12.41% |
| | Seq(GSE11431)/Homer | | | | | | | |
| 49 | Bachl(bZIP)/K562-Bach1-ChIP- | 1.00E−08 | −1.92E+01 | 0 | 166 | 3.07% | 10047.1 | 1.90% |
| | Seq(GSE31477)/Homer | | | | | | | |
| 50 | MafK(bZIP)/C2C12-MafK-ChIP- | 1.00E−08 | −1.87E+01 | 0 | 424 | 7.84% | 31305.6 | 5.93% |
| | Seq(GSE36030)/Homer | | | | | | | |
| 51 | NF-E2(bZIP)/K562-NFE2-ChIP- | 1.00E−08 | −1.85E+01 | 0 | 171 | 3.16% | 10537.3 | 2.00% |
| | Seq(GSE31477)/Homer | | | | | | | |
| 52 | Foxo3(Forkhead)/U2OS-Foxo3- | 1.00E−07 | −1.73E+01 | 0 | 1204 | 22.27% | 101868.9 | 19.30% |
| | ChIP-Seq(E-MTAB-2701)/Homer | | | | | | | |
| 53 | FOXK2(Forkhead)/U2OS- | 1.00E−07 | −1.68E+01 | 0 | 1054 | 19.50% | 88300.6 | 16.73% |
| | FOXK2-ChIP-Seq(E-MTAB- | | | | | | | |
| | 2204)/Homer | | | | | | | |
| 54 | TEAD4(TEA)/Tropoblast-Tead4- | 1.00E−07 | −1.62E+01 | 0 | 1292 | 23.90% | 110622.8 | 20.96% |
| | ChIP-Seq(GSE37350)/Homer | | | | | | | |
| 55 | Klf4(Zf)/mES-Klf4-ChIP- | 1.00E−06 | −1.53E+01 | 0 | 743 | 13.74% | 60642.2 | 11.49% |
| | Seq(GSE11431)/Homer | | | | | | | |
| 56 | NFIL3(bZIP)/HepG2-NFIL3- | 1.00E−06 | −1.53E+01 | 0 | 969 | 17.92% | 81225.4 | 15.39% |
| | ChIP-Seq(Encode)/Homer | | | | | | | |
| 57 | FOXP1(Forkhead)/H9-FOXP1- | 1.00E−06 | −1.52E+01 | 0 | 777 | 14.37% | 63768.3 | 12.08% |
| | ChIP-Seq(GSE31006)/Homer | | | | | | | |
| 58 | SPDEF(ETS)/VCaP-SPDEF- | 1.00E−06 | −1.49E+01 | 0 | 1480 | 27.38% | 128901.8 | 24.42% |
| | ChIP-Seq(SRA014231)/Homer | | | | | | | |
| 59 | USF1(bHLH)/GM12878-Usf1- | 1.00E−06 | −1.41E+01 | 0 | 699 | 12.93% | 57180.6 | 10.83% |
| | ChIP-Seq(GSE32465)/Homer | | | | | | | |
| 60 | Bcl6(Zf)/Liver-Bcl6-ChIP- | 1.00E−05 | −1.34E+01 | 0 | 1825 | 33.76% | 162504.3 | 30.79% |
| | Seq(GSE31578)/Homer | | | | | | | |
| 61 | EWS:ERG- | 1.00E−05 | −1.33E+01 | 0 | 1106 | 20.46% | 94900.5 | 17.98% |
| | fusion(ETS)/CADO_ES1- | | | | | | | |
| | EWS:ERG-ChIP- | | | | | | | |
| | Scq(SRA014231)/Homer | | | | | | | |
| 62 | FoxD3(forkhead)/ZebrafishEmbry | 1.00E−05 | −1.32E+01 | 0 | 1336 | 24.71% | 116442 | 22.06% |
| | o-Foxd3.biotin-ChIP- | | | | | | | |
| | seq(GSE106676)/Homer | | | | | | | |
| 63 | TEAD2(TEA)/Py2T-Tead2-ChIP- | 1.00E−05 | −1.31E+01 | 0 | 837 | 15.48% | 70211.3 | 13.30% |
| | Seq(GSE55709)/Homer | | | | | | | |
| 64 | KLF6(Zf)/PDAC-KLF6-ChIP- | 1.00E−05 | −1.22E+01 | 0 | 1726 | 31.93% | 153939.7 | 29.17% |
| | Seq(GSE64557)/Homer | | | | | | | |
| 65 | bZIP:IRF(bZIP, IRF)/Th17-BatF- | 1.00E−05 | −1.18E+01 | 0 | 659 | 12.19% | 54582.5 | 10.34% |
| | ChIP-Seq(GSE39756)/Homer | | | | | | | |
| 66 | Stat3+il21(Stat)/CD4-Stat3-ChIP- | 1.00E−05 | −1.16E+01 | 0.0001 | 1044 | 19.31% | 90110.2 | 17.07% |
| | Seq(GSE19198)/Homer | | | | | | | |
| 67 | IRF3(IRF)/BMDM-Irf3-ChIP- | 1.00E−04 | −1.13E+01 | 0.0001 | 512 | 9.47% | 41582.3 | 7.88% |
| | Seq(GSE67343)/Homer | | | | | | | |
| 68 | CLOCK(bHLH)/Liver-Clock- | 1.00E−04 | −1.11E+01 | 0.0001 | 765 | 14.15% | 64607.5 | 12.24% |
| | ChIP-Seq(GSE39860)/Homer | | | | | | | |
| 69 | Tlx?(NR)/NPC-H3K4me1-ChIP- | 1.00E−04 | −1.11E+01 | 0.0001 | 796 | 14.72% | 67471.2 | 12.78% |
| | Scq(GSE16256)/Homer | | | | | | | |
| 70 | TEAD1(TEAD)/HepG2-TEAD1- | 1.00E−04 | −1.05E+01 | 0.0002 | 1395 | 25.80% | 123772.9 | 23.45% |
| | ChIP-Seq(Encode)/Homer | | | | | | | |
| 71 | NF1:FOXA1(CTF, Forkhead)/LN | 1.00E−04 | −1.04E+01 | 0.0002 | 123 | 2.28% | 8183.5 | 1.55% |
| | CAP-FOXA1-ChIP- | | | | | | | |
| | Seq(GSE27824)/Homer | | | | | | | |
| 72 | NFE2L2(bZIP)/HepG2-NFE2L2- | 1.00E−04 | −1.03E+01 | 0.0002 | 130 | 2.40% | 8768.6 | 1.66% |
| | ChIP-Seq(Encode)/Homer | | | | | | | |
| 73 | NRF1(NRF)/MCF7-NRF1-ChIP- | 1.00E−04 | −1.01E+01 | 0.0002 | 185 | 3.42% | 13345.2 | 2.53% |
| | Seq(Unpublished)/Homer | | | | | | | |
| 74 | MITF(bHLH)/MastCells-MITF- | 1.00E−04 | −1.01E+01 | 0.0003 | 1281 | 23.70% | 113304.2 | 21.47% |
| | ChIP-Seq(GSE48085)/Homer | | | | | | | |
| 75 | TEAD3(TEA)/HepG2-TEAD3- | 1.00E−04 | −9.69E+00 | 0.0004 | 1574 | 29.12% | 141317.7 | 26.78% |
| | ChIP-Seq(Encode)/Homer | | | | | | | |
| 76 | c-Myc(bHLH)/LNCAP-cMyc- | 1.00E−04 | −9.49E+00 | 0.0004 | 634 | 11.73% | 53470.1 | 10.13% |
| | ChIP-Seq(Unpublished)/Homer | | | | | | | |
| 77 | IRF8(IRF)/BMDM-IRF8-ChIP- | 1.00E−04 | −9.36E+00 | 0.0005 | 506 | 9.36% | 41886.8 | 7.94% |
| | Seq(GSE77884)/Homer | | | | | | | |
| 78 | BMAL1(bHLH)/Liver-Bmall- | 1.00E−04 | −9.22E+00 | 0.0006 | 2163 | 40.01% | 198138.7 | 37.54% |
| | ChIP-Seq(GSE39860)/Homer | | | | | | | |
| 79 | HLF(bZIP)/HSC-HLF.Flag-ChIP- | 1.00E−03 | −9.13E+00 | 0.0006 | 1163 | 21.51% | 102855.6 | 19.49% |
| | Seq(GSE69817)/Homer | | | | | | | |
| 80 | STAT4(Stat)/CD4-Stat4-ChIP- | 1.00E−03 | −9.07E+00 | 0.0006 | 1326 | 24.53% | 118270.2 | 22.41% |
| | Seq(GSE22104)/Homer | | | | | | | |
| 81 | Usf2(bHLH)/C2C12-Usf2-ChIP- | 1.00E−03 | −9.01E+00 | 0.0007 | 543 | 10.04% | 45414 | 8.60% |
| | Seq(GSE36030)/Homer | | | | | | | |
| 82 | Sp5(Zf)/mES-Sp5.Flag-ChIP- | 1.00E−03 | −8.77E+00 | 0.0008 | 1616 | 29.89% | 146049.6 | 27.67% |
| | Seq(GSE72989)/Homer | | | | | | | |
| 83 | IRF2(IRF)/Erythroblas-IRF2- | 1.00E−03 | −8.73E+00 | 0.0009 | 197 | 3.64% | 14752.3 | 2.80% |
| | ChIP-Seq(GSE36985)/Homer | | | | | | | |
| 84 | TFE3(bHLH)/MEF-TFE3-ChIP- | 1.00E−03 | −8.72E+00 | 0.0009 | 136 | 2.52% | 9603 | 1.82% |
| | Seq(GSE75757)/Homer | | | | | | | |
| 85 | Max(bHLH)/K562-Max-ChIP- | 1.00E−03 | −8.56E+00 | 0.001 | 903 | 16.70% | 78887.2 | 14.95% |
| | Seq(GSE31477)/Homer | | | | | | | |
| 86 | PU.1(ETS)/ThioMac-PU.1-ChIP- | 1.00E−03 | −8.55E+00 | 0.001 | 732 | 13.54% | 63016.8 | 11.94% |
| | Seq(GSE21512)/Homer | | | | | | | |
| 87 | CEBP(bZIP)/ThioMac-CEBPb- | 1.00E−03 | −7.93E+00 | 0.0018 | 1052 | 19.46% | 93289 | 17.68% |
| | ChIP-Seq(GSE21512)/Homer | | | | | | | |
| 88 | Rfx2(HTH)/LoVo-RFX2-ChIP- | 1.00E−03 | −7.74E+00 | 0.0022 | 158 | 2.92% | 11705.2 | 2.22% |
| | Seq(GSE49402)/Homer | | | | | | | |
| 89 | MNT(bHLH)/HepG2-MNT-ChIP- | 1.00E−03 | −7.58E+00 | 0.0025 | 1297 | 23.99% | 116705.4 | 22.11% |
| | Seq(Encode)/Homer | | | | | | | |
| 90 | Ets1-distal(ETS)/CD4+-PolII- | 1.00E−03 | −7.52E+00 | 0.0027 | 461 | 8.53% | 38711.7 | 7.33% |
| | ChIP-Seq(Barski_et_al.)/Homer | | | | | | | |
| 91 | ISRE(IRF)/ThioMac-LPS- | 1.00E−03 | −7.41E+00 | 0.0029 | 124 | 2.29% | 8920.2 | 1.69% |
| | Expression(GSE23622)/Homer | | | | | | | |
| 92 | TEAD(TEA)/Fibroblast-PU.1- | 1.00E−03 | −7.40E+00 | 0.0029 | 1002 | 18.53% | 88985.4 | 16.86% |
| | ChIP-Seq(Unpublished)/Homer | | | | | | | |
| 93 | MafA(bZIP)/Islet-MafA-ChIP- | 1.00E−03 | −7.37E+00 | 0.003 | 1102 | 20.38% | 98426 | 18.65% |
| | Seq(GSE30298)/Homer | | | | | | | |
| 94 | MafB(bZIP)/BMM-Mafb-ChIP- | 1.00E−03 | −7.36E+00 | 0.003 | 636 | 11.76% | 54865.2 | 10.40% |
| | Seq(GSE75722)/Homer | | | | | | | |
| 95 | GRE(NR), IR3/RAW264.7-GRE- | 1.00E−03 | −7.35E+00 | 0.003 | 351 | 6.49% | 28836.8 | 5.46% |
| | ChIP-Seq(Unpublished)/Homer | | | | | | | |
| 96 | Klf9(Zf)/GBM-Klf9-ChIP- | 1.00E−03 | −7.26E+00 | 0.0032 | 665 | 12.30% | 57610.5 | 10.92% |
| | Seq(GSE62211)/Homer | | | | | | | |
| 97 | PU.1:IRF8(ETS:IRF)/pDC-Irf8- | 1.00E−02 | −6.89E+00 | 0.0046 | 307 | 5.68% | 25088.3 | 4.75% |
| | ChIP-Seq(GSE66899)/Homer | | | | | | | |
| 98 | HNF1b(Homeobox)/PDAC- | 1.00E−02 | −6.85E+00 | 0.0048 | 272 | 5.03% | 21976.8 | 4.16% |
| | HNF1B-ChIP- | | | | | | | |
| | Seq(GSE64557)/Homer | | | | | | | |
| 99 | CEBP:AP1(bZIP)/ThioMac- | 1.00E−02 | −6.79E+00 | 0.005 | 1031 | 19.07% | 92192.3 | 17.47% |
| | CEBPb-ChIP- | | | | | | | |
| | Seq(GSE21512)/Homer | | | | | | | |
| 100 | STAT5(Stat)/mCD4+-Stat5-ChIP- | 1.00E−02 | −6.73E+00 | 0.0053 | 493 | 9.12% | 42069 | 7.97% |
| | Seq(GSE12346)/Homer | | | | | | | |
| 101 | E-box(bHLH)/Promoter/Homer | 1.00E−02 | −6.65E+00 | 0.0057 | 128 | 2.37% | 9457.3 | 1.79% |
| 102 | RFX(HTH)/K562-RFX3-ChIP- | 1.00E−02 | −6.55E+00 | 0.0062 | 139 | 2.57% | 10423.5 | 1.97% |
| | Seq(SRA012198)/Homer | | | | | | | |
| 103 | NRF(NRF)/Promoter/Homer | 1.00E−02 | −6.52E+00 | 0.0063 | 210 | 3.88% | 16611.8 | 3.15% |
| 104 | Hnf1(Homeobox)/Liver-Foxa2- | 1.00E−02 | −6.33E+00 | 0.0075 | 243 | 4.50% | 19607.2 | 3.72% |
| | Chip-Seq(GSE25694)/Homer | | | | | | | |
| 105 | Rfx1(HTH)/NPC-H3K4me1- | 1.00E−02 | −6.30E+00 | 0.0077 | 306 | 5.66% | 25268 | 4.79% |
| | ChIP-Seq(GSE16256)/Homer | | | | | | | |
| 106 | IRF1(IRF)/PBMC-IRF1-ChIP- | 1.00E−02 | −6.28E+00 | 0.0077 | 239 | 4.42% | 19271.7 | 3.65% |
| | Seq(GSE43036)/Homer | | | | | | | |
| 107 | AMYB(HTH)/Testes-AMYB- | 1.00E−02 | −6.28E+00 | 0.0077 | 1888 | 34.92% | 174446.2 | 33.05% |
| | ChIP-Seq(GSE44588)/Homer | | | | | | | |
| 109 | NPAS2(bHLH)/Liver-NPAS2- | 1.00E−02 | −6.22E+00 | 0.0081 | 1492 | 27.60% | 136507.2 | 25.86% |
| | ChIP-Seq(GSE39860)/Homer | | | | | | | |
| 110 | Foxh1(Forkhead)/hESC-FOXH1- | 1.00E−02 | −6.21E+00 | 0.0081 | 807 | 14.93% | 71583.5 | 13.56% |
| | ChIP-Seq(GSE29422)/Homer | | | | | | | |
| 111 | ARE(NR)/LNCAP-AR-ChIP- | 1.00E−02 | −5.98E+00 | 0.0101 | 373 | 6.90% | 31505.9 | 5.97% |
| | Seq(GSE27824)/Homer | | | | | | | |
| 112 | MYB(HTH)/ERMYB-Myb- | 1.00E−02 | −5.96E+00 | 0.0101 | 2086 | 38.59% | 193891.5 | 36.74% |
| | ChIPSeq(GSE22095)/Homer | | | | | | | |
| 113 | PAX5(Paired, Homeobox), condensed/ | 1.00E−02 | −5.94E+00 | 0.0102 | 167 | 3.09% | 13043.4 | 2.47% |
| | GM12878-PAX5-ChIP- | | | | | | | |
| | Seq(GSE32465)/Homer | | | | | | | |
| 114 | Cdx2(Homeobox)/mES-Cdx2- | 1.00E−02 | −5.87E+00 | 0.0109 | 972 | 17.98% | 87391.5 | 16.56% |
| | ChIP-Seq(GSE14586)/Homer | | | | | | | |
| 115 | CDX4(Homeobox)/ZebrafishEmbryos- | 1.00E−02 | −5.63E+00 | 0.0138 | 1209 | 22.36% | 110088.2 | 20.86% |
| | Cdx4.Myc-ChIP- | | | | | | | |
| | Seq(GSE48254)/Homer | | | | | | | |
| 116 | Six2(Homeobox)/NephronProgenitor- | 1.00E−02 | −5.57E+00 | 0.0145 | 1239 | 22.92% | 113000.2 | 21.41% |
| | Six2-ChIP- | | | | | | | |
| | Seq(GSE39837)/Homer | | | | | | | |
| 117 | PGR(NR)/EndoStromal-PGR- | 1.00E−02 | −5.56E+00 | 0.0145 | 344 | 6.36% | 29083.2 | 5.51% |
| | ChIP-Seq(GSE69539)/Homer | | | | | | | |
| 118 | Maz(Zf)/HepG2-Maz-ChIP- | 1.00E−02 | −5.47E+00 | 0.0157 | 1826 | 33.78% | 169359.3 | 32.09% |
| | Seq(GSE31477)/Homer | | | | | | | |
| 119 | Rfx6(HTH)/Min6b1-Rfx6.HA- | 1.00E−02 | −5.44E+00 | 0.016 | 1461 | 27.03% | 134331.6 | 25.45% |
| | ChIP-Seq(GSE62844)/Homer | | | | | | | |
| 120 | KLF10(Zf)/HEK293- | 1.00E−02 | −5.43E+00 | 0.0161 | 894 | 16.54% | 80406.1 | 15.23% |
| | KLF10.GFP-ChIP- | | | | | | | |
| | Seq(GSE58341)/Homer | | | | | | | |
| 121 | IRF:BATF(IRF:bZIP)/pDC-Irf8- | 1.00E−02 | −5.13E+00 | 0.0215 | 177 | 3.27% | 14225 | 2.70% |
| | ChIP-Seq(GSE66899)/Homer | | | | | | | |
| 122 | Smad3(MAD)/NPC-Smad3-ChIP- | 1.00E−02 | −5.06E+00 | 0.0228 | 3247 | 60.06% | 308140.1 | 58.39% |
| | Seq(GSE36673)/Homer | | | | | | | |
| 123 | Sox3(HMG)/NPC-Sox3-ChIP- | 1.00E−02 | −5.03E+00 | 0.0234 | 2134 | 39.47% | 199628.2 | 37.82% |
| | Seq(GSE33059)/Homer | | | | | | | |
| 124 | bHLHE41(bHLH)/proB-Bhlhe41- | 1.00E−02 | −4.96E+00 | 0.0249 | 1416 | 26.19% | 130541.3 | 24.73% |
| | ChIP-Seq(GSE93764)/Homer | | | | | | | |
| 125 | Tcfcp211(CP2)/mES-Tcfcp211- | 1.00E−02 | −4.96E+00 | 0.0249 | 218 | 4.03% | 17964.8 | 3.40% |
| | ChIP-Seq(GSE11431)/Homer | | | | | | | |
| 126 | PRDM1(Zf)/Hela-PRDM1-ChIP- | 1.00E−02 | −4.92E+00 | 0.0255 | 771 | 14.26% | 69246.4 | 13.12% |
| | Seq(GSE31477)/Homer | | | | | | | |
| 127 | Foxo1(Forkhead)/RAW-Foxo1- | 1.00E−02 | −4.84E+00 | 0.0273 | 2427 | 44.89% | 228296.5 | 43.26% |
| | ChIP-Seq(Fan_et_al.)/Homer | | | | | | | |
| 128 | n-Myc(bHLH)/mES-nMyc-ChIP- | 1.00E−02 | −4.74E+00 | 0.0301 | 903 | 16.70% | 81884.5 | 15.52% |
| | Seq(GSE11431)/Homer | | | | | | | |
| 129 | GFY-Staf(?, Zf)/Promoter/Homer | 1.00E−02 | −4.72E+00 | 0.0303 | 78 | 1.44% | 5732.1 | 1.09% |
| 130 | Rfx5(HTH)/GM12878-Rfx5- | 1.00E−02 | −4.71E+00 | 0.0304 | 451 | 8.34% | 39452.5 | 7.48% |
| | ChIP-Seq(GSE31477)/Homer | | | | | | | |
| 131 | CREB5(bZIP)/LNCaP- | 1.00E−02 | −4.68E+00 | 0.0312 | 542 | 10.03% | 47950.6 | 9.09% |
| | CREB5.V5-ChIP- | | | | | | | |
| | Seq(GSE137775)/Homer | | | | | | | |
| 132 | NFkB-p65-Rel(RHD)/ThioMac- | 1.00E−02 | −4.64E+00 | 0.0323 | 112 | 2.07% | 8683.7 | 1.65% |
| | LPS- | | | | | | | |
| | Expression(GSE23622)/Homer | | | | | | | |

*% of Background Sequences with Motif

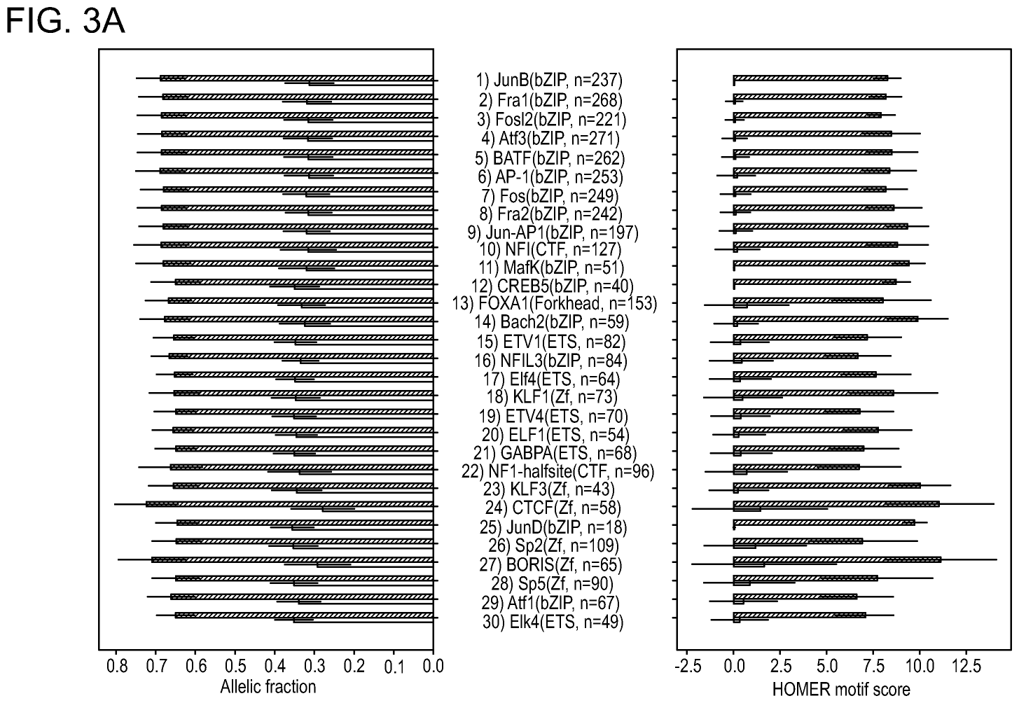

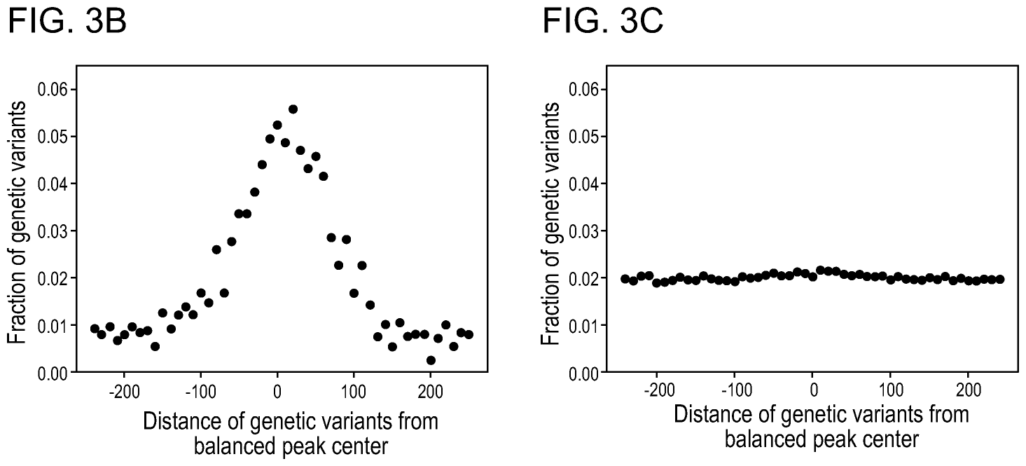

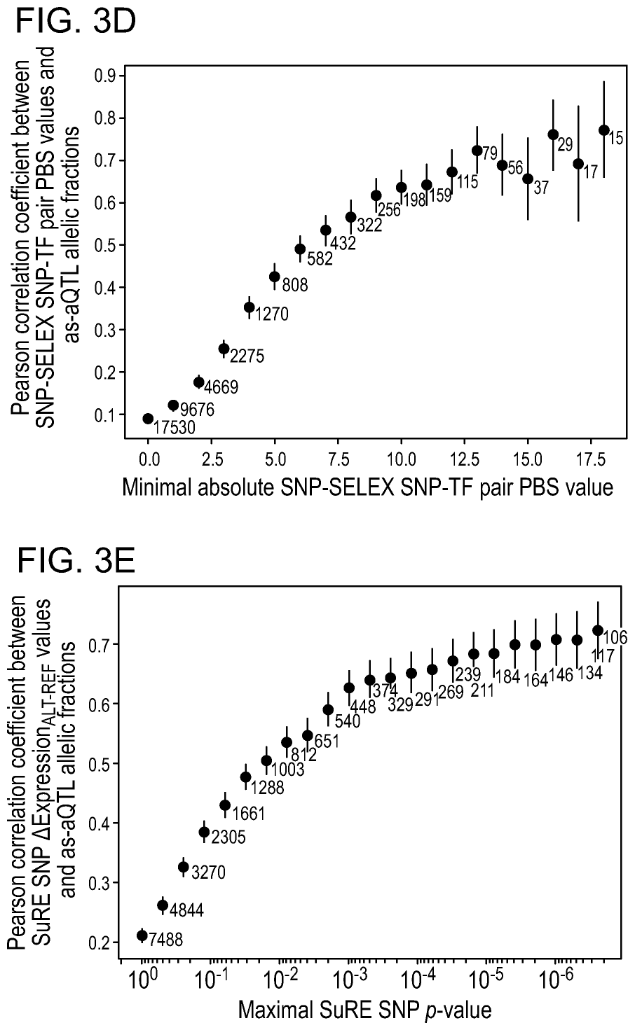

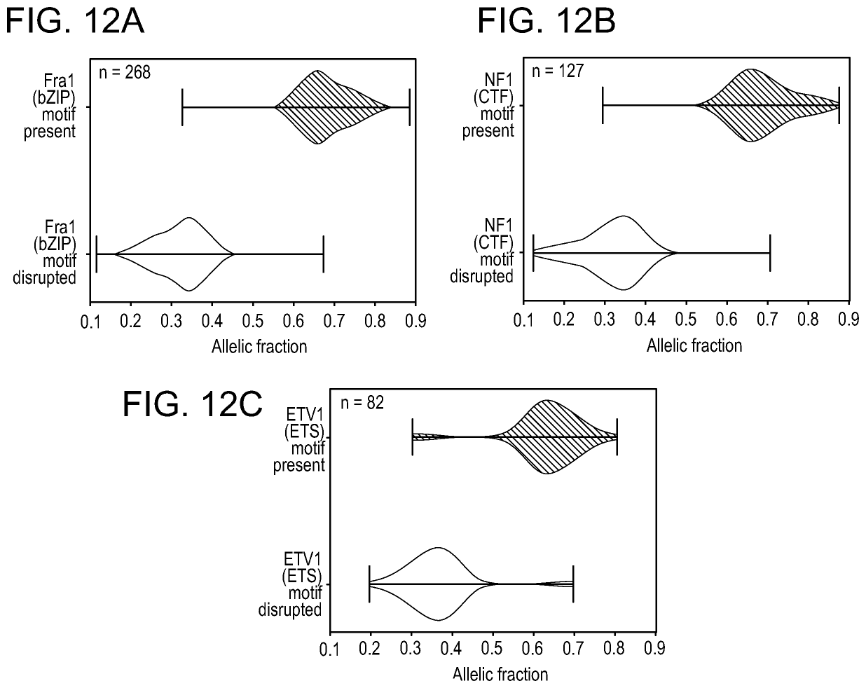

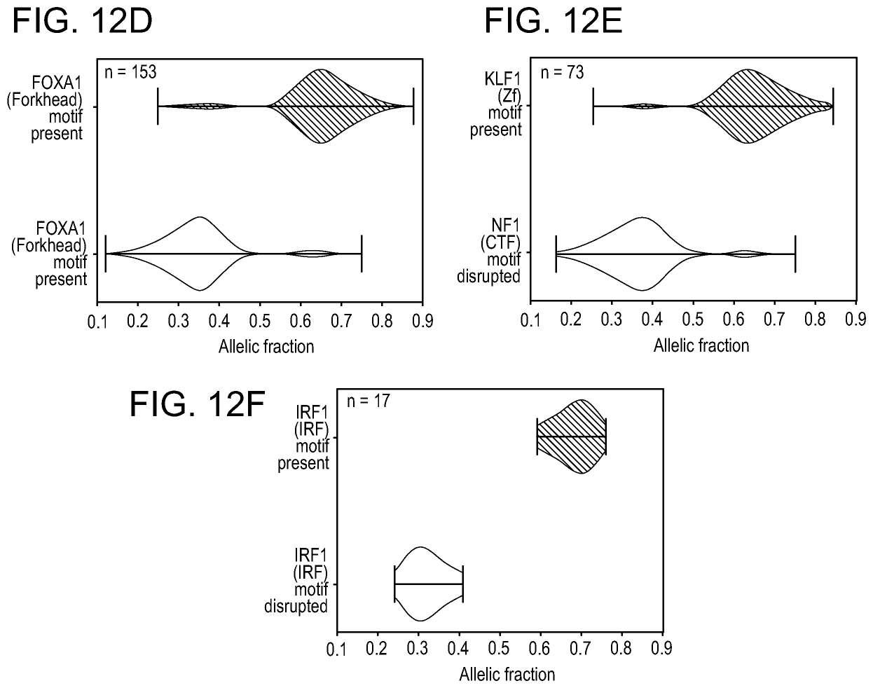

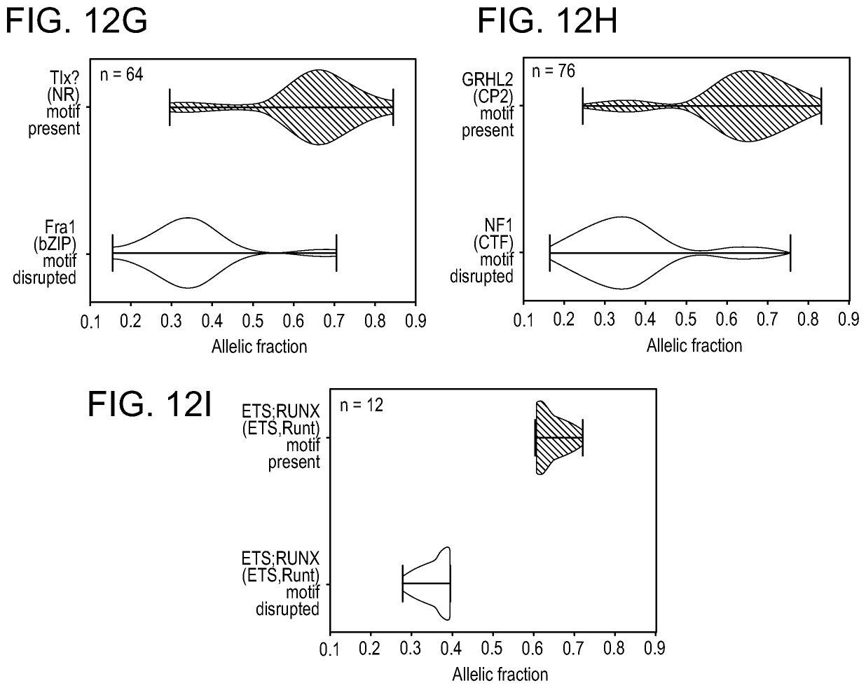

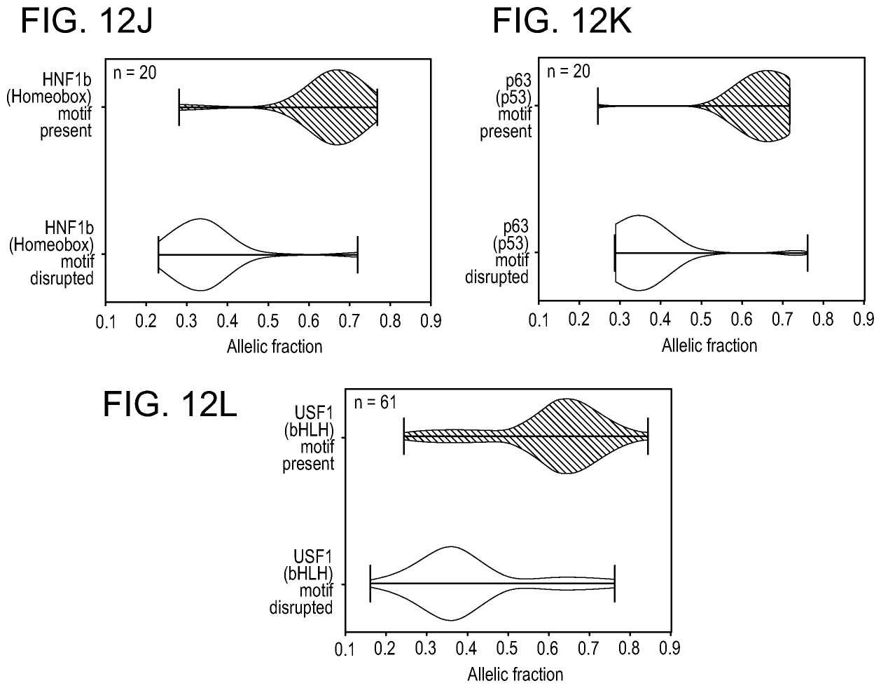

Next, 2,868 pan-cancer asQTL peaks containing a single genetic variant were focused on, to look for synergy between TF motif alteration and allelic accessibility. It was reasoned that such synergy would be evidence of the as-aQTL variant influencing accessibility by affecting TF binding. 151 TF motifs were identified with a statistically significant difference in motif scores between sequences with higher and lower allelic fractions (5% FDR; FIG. 3A, FIG. 12A-FIG. 12L, FIG. 13A, Table 5B, and Table 5C). For nearly all TF motifs, higher allelic fractions were observed at sequences with higher-scoring TF motifs, implying that the allele increasing TF motif scores leads to an allele-specific increase in accessibility, likely through an allele-specific increase in TF binding. In total, the motif analysis identified TF motifs that were significantly altered by a genetic variant (motif score A >3) at 2,390 out of 2,868 as-aQTLs (˜83%). Of note, ˜73% of allelically balanced (119,642 out of 162,595) peaks (i.e., those for which as-aQTL analysis was not significant) contained significantly altered TF motifs, due to the nearly ubiquitous presence of TF motifs in accessible regions. However, while motif-altering genetic variants inside as-aQTLs were enriched near the center of the peak, motif-altering genetic variants inside balanced peaks were uniformly distributed (FIG. 3B-FIG. 3C). The majority of as-aQTLs may thus be explained by genetic variants that directly alter known TF motifs, including motifs of TFs that are implicated in dysregulation in cancer, though the breadth of enriched TFs suggests multifactorial relationships between binding and accessibility.

### Example 6: Differential TF Binding is Observed at Cancer as-aQTLs

To validate differential TF binding at as-aQTLs it was examined whether as-aQTLs harbor genetic variants that have been found to significantly affect TF binding in a high-throughput multiplex protein-DNA binding assay (SNP-SELEX) (Yan, J. et al., Nature (2021)). The SNP-SELEX study examined 150,411 genetic variants and 2,660,658 genetic variant-TF pairs for differential TF binding in vitro. Whether there was a directional relationship between allelic fractions at pan-cancer as-aQTL variants and SNP-SELEX SNP-TF pair Preferential Binding Scores (PBSs) was investigated. Intersecting 10,005 genetic variants from 5,406 pan-cancer as-aQTLs with 2,660,658 SNP-TF pairs yielded 1,267 genetic variants and 17,532 SNP-TF pairs. The as-aQTL allele fractions were significantly correlated with PBS values, reaching a Pearson correlation of 0.77 (15 SNP-TF pairs; p=7.6×10⁻⁵) for SNP-TF pairs with the largest absolute PBS values (FIG. 3D). Alleles with more reads (more open chromatin) were associated with increased TF binding. Individually, 108 as-aQTL SNP-TF pairs were significant after Bonferroni correction of the SNP-SELEX p-value, out of which 79 validated with consistent sign (sign test p=1.6×10⁻⁶). These results demonstrate a highly significant, directional correlation between in vivo allelic imbalance and high-confidence in vitro transcription factor binding, experimentally confirming differential TF binding at as-aQTLs.

### Example 7: Cancer as-aQTLs Cause Differential Gene Expression

A similar analysis was conducted to validate the effect of as-aQTLs on gene expression measured with the high-throughput survey of regulatory elements (SuRE) reporter technology (van Arensbergen, J. et al., Nat. Biotechnol. 35, 145-153 (2017)). A recent study conducted SuRE measurements for 5,919,293 SNPs which we intersected with our 10,005 genetic variants from 5,406 pan-cancer as-aQTLs, which yielded 7,554 genetic variants. The as-aQTL allele fractions were significantly correlated with differential gene expression, reaching a Pearson correlation of 0.72 (p=2.4×10⁻¹⁸) for the most significant SuRE SNPs (FIG. 3E). Alleles with more reads (more open chromatin) were associated with higher gene expression. Individually, 179 as-aQTL variants were significant SuRE SNPs after Bonferroni correction of the SuRE SNPs p-value, out of which 177 validated with consistent sign (sign test p=4.2×10⁻⁵⁰). Notably, while the correlation between as-aQTL allelic fractions and TF binding/gene expression measurements increased with more stringent significance thresholds on the experimental data, the correlation was not substantially influenced by more stringent as-aQTL thresholds (FIG. 13B and FIG. 13C), indicating that the as-aQTL false positive rate was well controlled.

### Example 8: Regulome-Wide Association Study Links Peaks to Cancer Risk

Finally, individual genetically driven accessible regions that were associated with cancer risk were identified, by building predictive models of accessibility and imputing them into cancer GWAS data. This analysis was termed Regulome-Wide Association Study (RWAS), as it is analogous to the Transcriptome-Wide Association Study (TWAS) (Gusev, A. et al., Nat. Genet. 48, 245-252 (2016)) but trained on TF binding-induced accessibility rather than transcriptional phenotypes. RWAS power was also increased by incorporating signals from both total accessibility and allelic imbalance information, as has been recently proposed for QTL fine-mapping (Wang, A. T. et al., Am. J. Hum. Genet. 106, 170-187 (2020)) and TWAS (Liang, Y. et al., Nat. Commun. 12, 1424 (2021)).

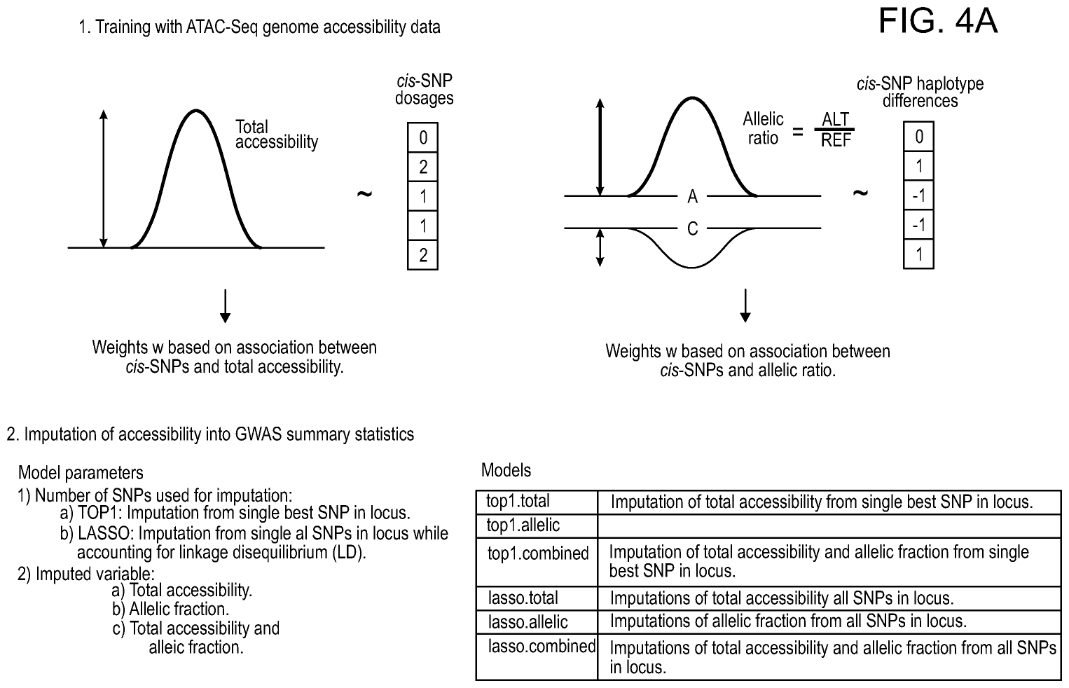

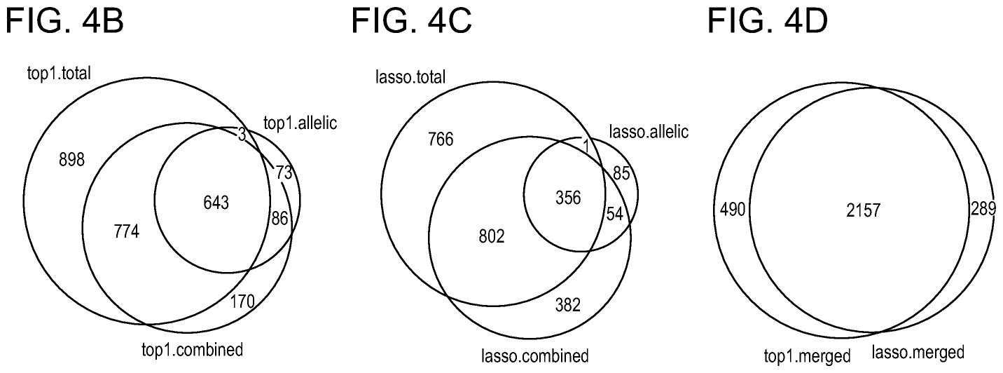

The first step of an RWAS is constructing accurate genetic predictors of each accessibility feature (FIG. 4A). The performance of six model types that predicted the accessibility phenotype from all SNPs in the cis-locus (lasso) or the single best SNP in the cis-locus (top1) using allelic, total, or combined signal (FIG. 4A) were investigated. In total, 2,936 unique significantly predictive features (by cross-validation) were identified with high concordance between all six model types (FIG. 4B-FIG. 4D). The top1.total model type performed best, identifying 2,318 predictable features, but incorporating models that use all SNPs in the locus and allelic activity identified 618 additional features with predictive models (˜27% increase). The large number of features identified by the top1.total model type was reflective of generally more models identifiable through the total activity phenotype which does not rely on heterozygous variants in the peak. Likewise, the top1 model type using a single causal variant generally outperformed the lasso model type using all SNPs which is in line with our previous observation that most as-aQTLs can likely be explained by the impact of a single genetic variant on a TF motif (Table 6A). Furthermore, TWAS model performance was compared with in breast cancer with all cancer types (Table 6B).

TABLE 6A
RWAS model performance based on cross-validation p-values computed for
allelic/total activity

| | | Training algorithm | | | | | |
|---|---|---|---|---|---|---|---|
| | | Lasso. | Lasso. | Lasso. | Top1. | Top1. | Top1. |
| | | total | allelic | combined | total | allelic | combined |
| All Models | Number | 397027 | 229675 | 212548 | 491036 | 257729 | 257729 |
| | models | | | | | | |
| | Average | 0.0112964 | 0.0282461 | 0.021023 | 0.0122549 | 0.0350192 | 0.0287098 |
| | cross* | | | | | | |
| Models with | Number | 49401 | 33698 | 37539 | 92580 | 46184 | 54757 |
| cross ^{†} | models | | | | | | |
| | Average | 0.0655303 | 0.138217 | 0.0824973 | 0.0471243 | 0.141779 | 0.0966059 |
| | cross* | | | | | | |
| Models with | Number | 1925 | 496 | 1594 | 2318 | 805 | 1673 |
| Bonferroni- | models | | | | | | |
| cross ^{‡} | Average | 0.182147 | 0.27337 | 0.164915 | 0.169371 | 0.208697 | 0.166671 |
| | cross* | | | | | | |

*Average cross-validation R2
^{†} Models with cross-validation p-values <0.05
^{‡} Models with significant Bonferroni-corrected cross-validation p-values

TABLE 6B
TWAS model performance for breast and prostate cancer based
on cross-validation p-values computed for total activity

| | | Breast Cancer | | Cancer | |
|---|---|---|---|---|---|
| | | Training algorithm | | Training algorithm | |
| | | lasso.total | top1.total | lasso.total | top1.total |
| All Models | Number of models | 4463 | 4463 | 4674 | 4674 |
| | Average cross- | 0.0433474 | 0.0401302 | 0.0767654 | 0.0714922 |
| | validation R2 | | | | |
| Models with | Number of models | 4012 | 3965 | 4196 | 4107 |
| cross-validation | Average cross- | 0.0480842 | 0.0450351 | 0.0852495 | 0.0810722 |
| p-value < 0.05 | validation R2 | | | | |
| Models with | Number of models | 2042 | 2006 | 2148 | 2075 |
| significant | Average cross- | 0.082335 | 0.0766575 | 0.142342 | 0.134777 |
| Bonferroni- | validation R2 | | | | |
| corrected | | | | | |
| cross-validation | | | | | |
| p-values | | | | | |

### Example 9: RWAS Implicates Hundreds of Cancer Risk Mechanisms

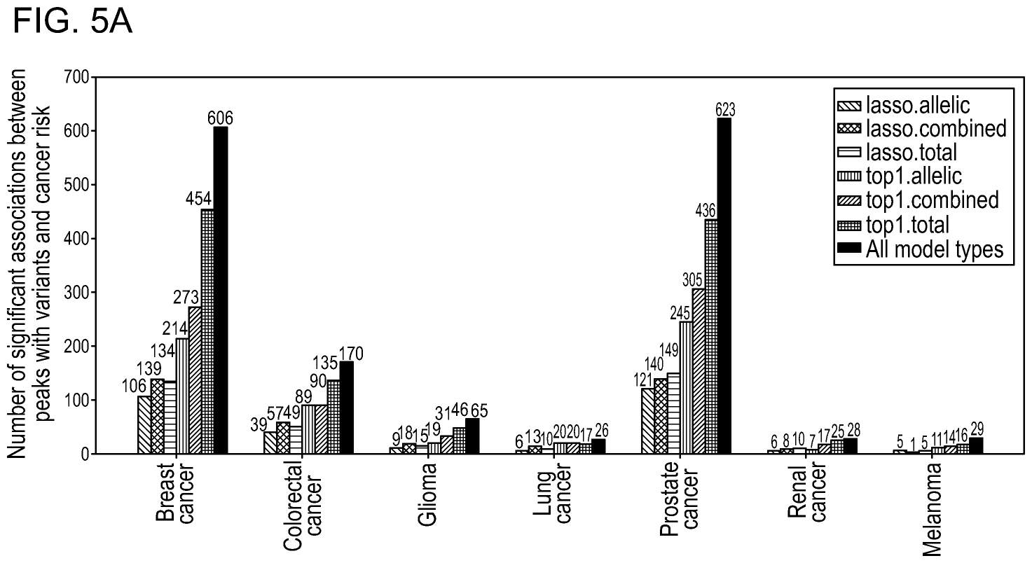

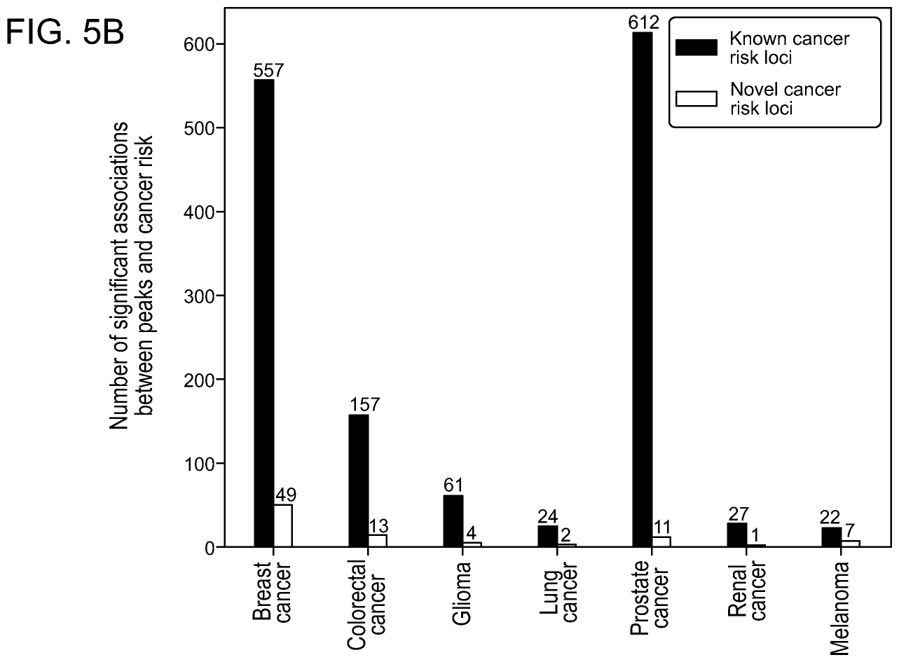

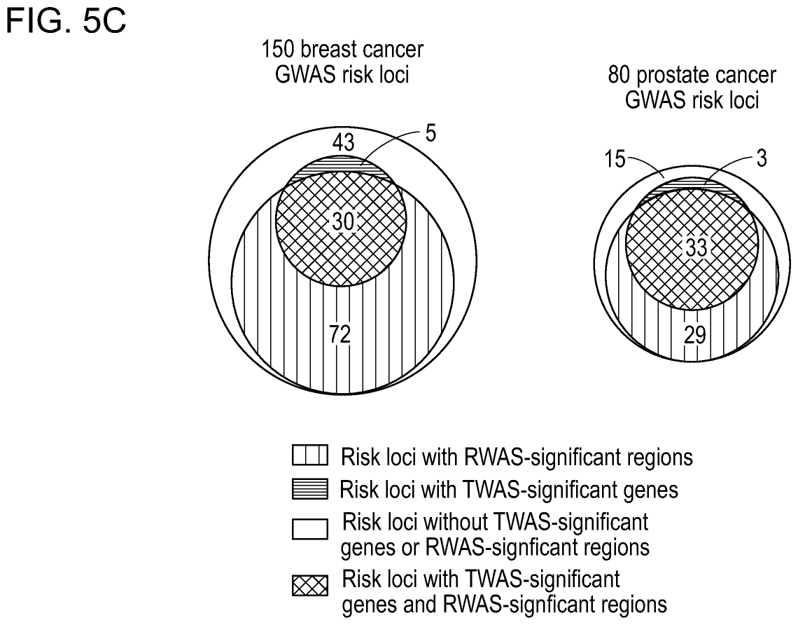

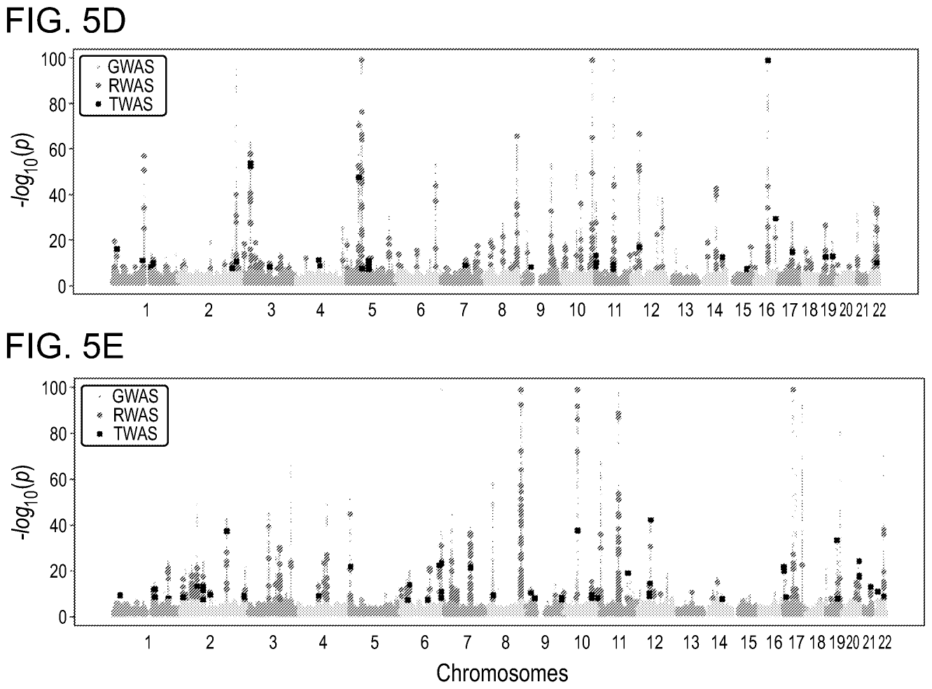

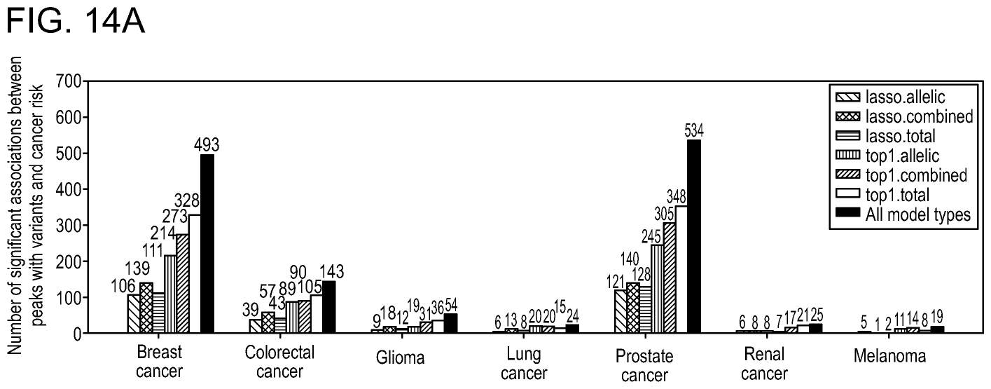

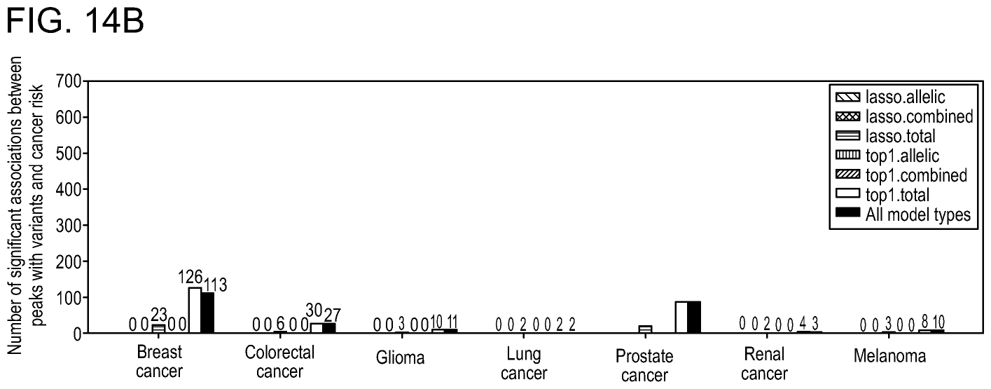

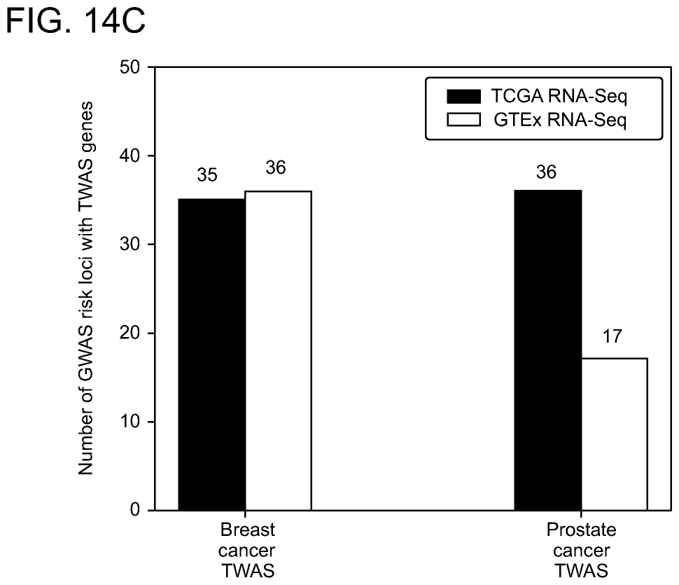

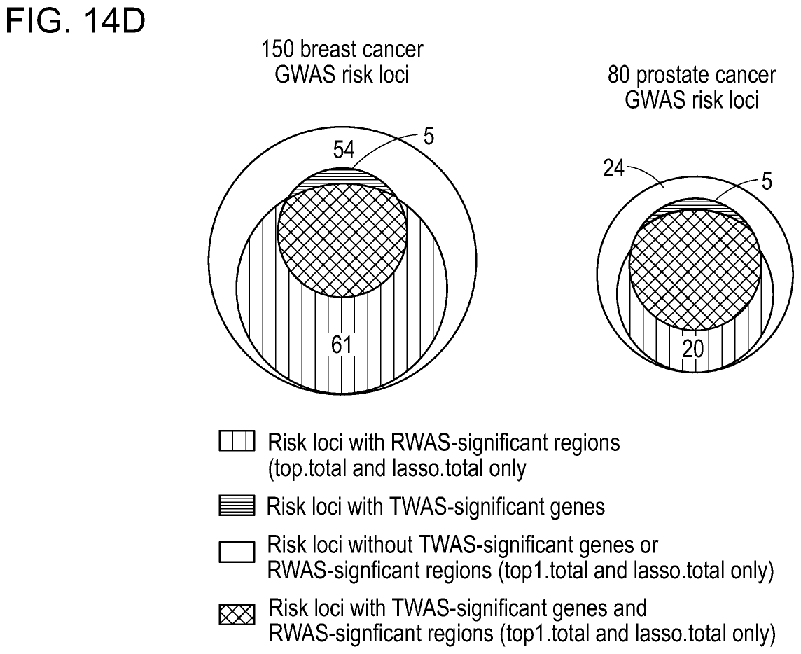

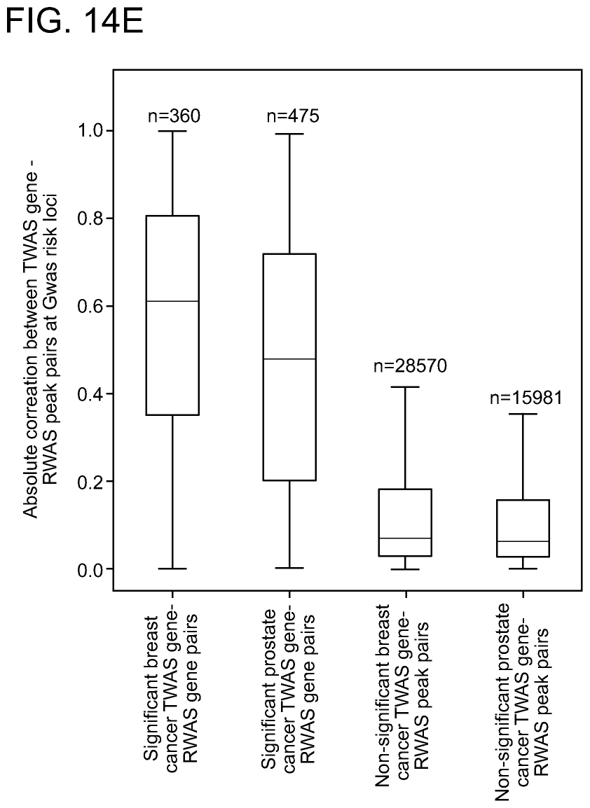

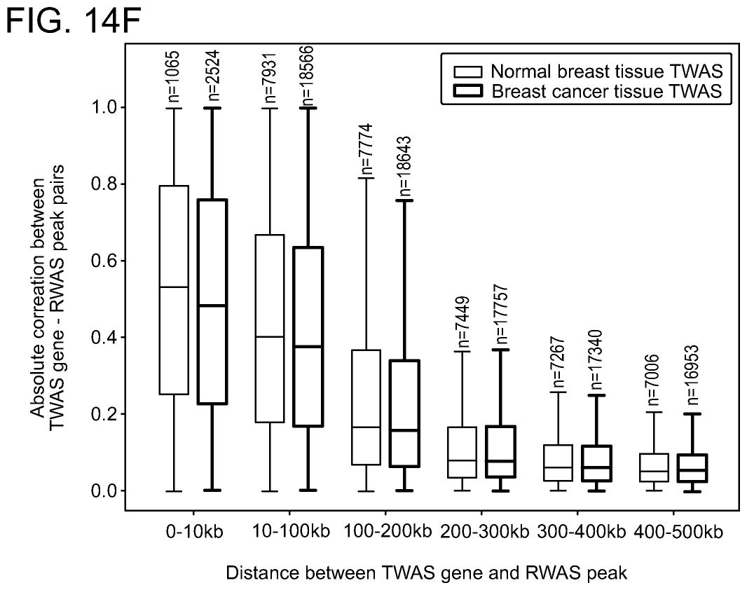

Having established robust predictive models, an RWAS of previously published GWAS summary statistics of seven cancer types was carried out. The cancer types included breast cancer (Zhang, H. et al., Nat. Genet. 52, 572-581 (2020)), colorectal cancer (Huyghe, J. R. et al., Nat. Genet. 51, 76-87 (2019)), prostate cancer (Schumacher, F. R. et al., Nat. Genet. 50, 928-936 (2018)), lung cancer (McKay, J. D. et al., Nat. Genet. 49, 1126-1132 (2017)), kidney cancer (Scelo, G. et al., Nat. Commun. 8, 15724 (2017)), glioma (Melin, B. S. et al., Nat. Genet. 49, 789-794 (2017)), and melanoma (Loh, P. R. et al., Nat. Genet. 50, 906-908 (2018)). A total of 1,547 Bonferroni significant RWAS associations across all cancer types were identified, using models with cross-validation p<0.05. The majority of RWAS associations, 606 and 623, were in breast and prostate cancer GWAS respectively, which had the largest GWAS sample size and the largest number of ATAC-seq samples (FIG. 5A). In agreement with the results of the model benchmarking, the top1. total model type consistently identified the most significant associations across cancers. However, on average, ˜40% more significant associations were discovered across all cancers when examining all model types, highlighting the utility of including multiple predictive model types. Notably, although peaks that harbored a genetic variant accounted for the majority of associations (FIG. 14A), 255/1,547 associated peaks contained no variant, suggesting indirect proximal causes (FIG. 14B). RWAS additionally identified 87 putatively “novel” associations at regions that did not reach significance in the original GWAS (GWAS p >5×10⁻⁸ for any variant within 1MB; FIG. 5B; Table 7A-Table 7G; RWAS results for 7 cancer types); with 49 in breast cancer and 11in prostate cancer. In total, approximately 10% of the discovered significant RWAS associations were not GWAS-significant, showcasing how RWAS can increase power at some loci, in addition to identifying putative mechanisms.

### Example 10: RWAS Outperforms TWAS and Links TWAS Genes to Regulatory Elements

The power of RWAS to identify associations at known GWAS risk loci relative to conventional TWAS analysis using TCGA cancer gene expression data (FIG. 5C) was evaluated. Using two studies that fine-mapped GWAS risk loci for breast and prostate cancer (Houlahan, K. E. et al., Nat. Med. 25, 1615-1626 (2019); Dadaev, T. et al., Nat. Commun. 9, 2256 (2018)), 102/150 (69%) of the breast cancer risk loci and 62/80 (78%) of the prostate cancer risk loci contained an RWAS signal (from any of the six model types). In comparison, only 35/150 (23%) breast and 36/80 (45%) prostate cancer risk loci contained a TWAS signal trained in corresponding cancer; despite larger TWAS training sizes. Similarly, 36/150 (24%) breast and 17/80 (21%) prostate cancer risk loci contained TWAS signal when using GTEx normal breast and prostate tissue expression (FIG. 14C, Table 6B). Furthermore, >85% of known loci that contained a TWAS significant gene also contained an RWAS significant peak (30/35 for breast cancer and 33/36 for prostate cancer; FIG. 5C), with the underlying models highly correlated relative to non-significant models (FIG. 6A-FIG. 6B, FIG. 15A-FIG. 15B, and FIG. 14E). Even among the significantly associated models, TWAS associations were generally less significant than RWAS associations (mean X_{TWAS}2 of 53.8 (breast) and 43.6 (prostate) vs mean X_{RWAS}² of 81.4 (breast) and 76.0 (prostate); FIG. 5D-FIG. 5E), highlighting the enrichment of RWAS models at loci with higher effect-size associations.

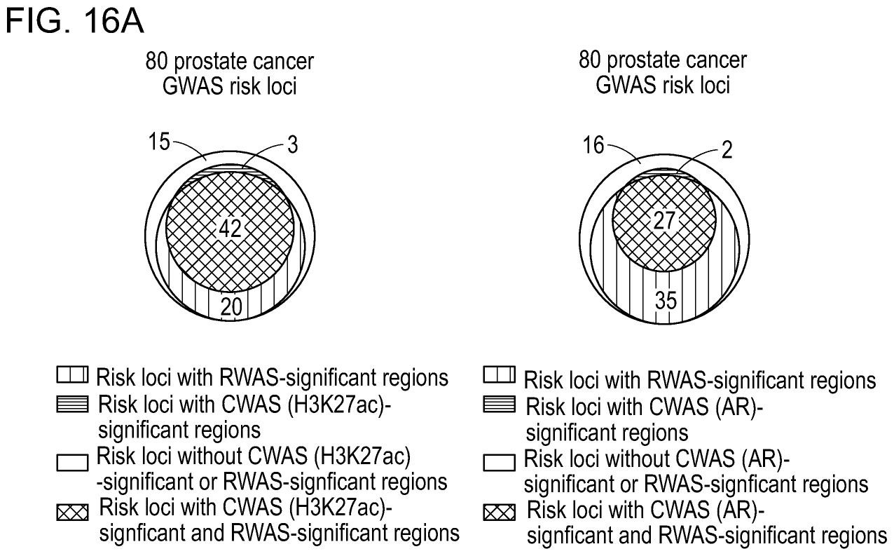

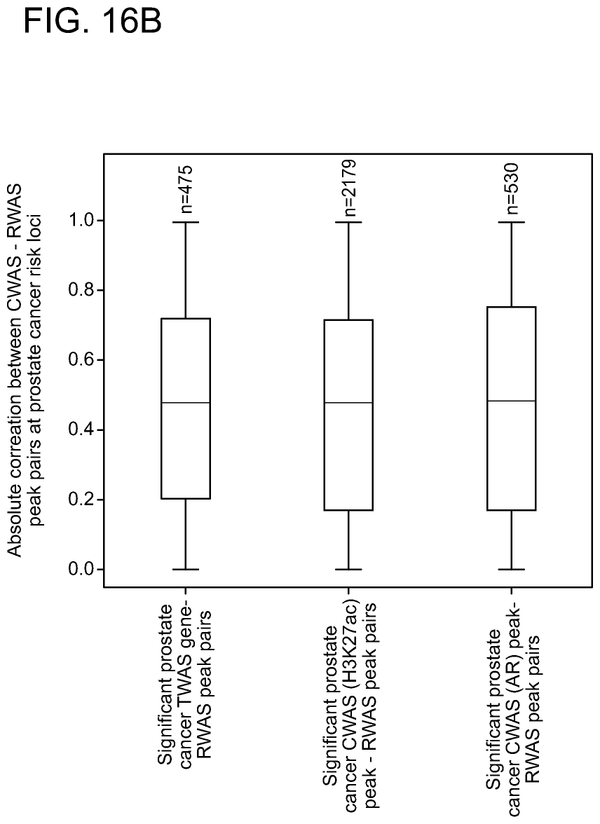

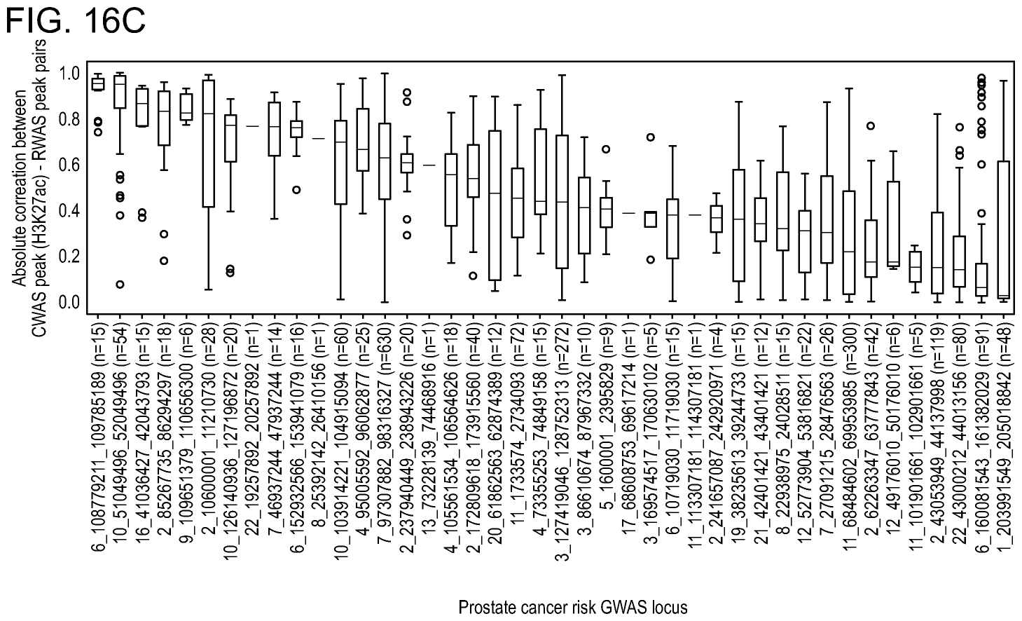

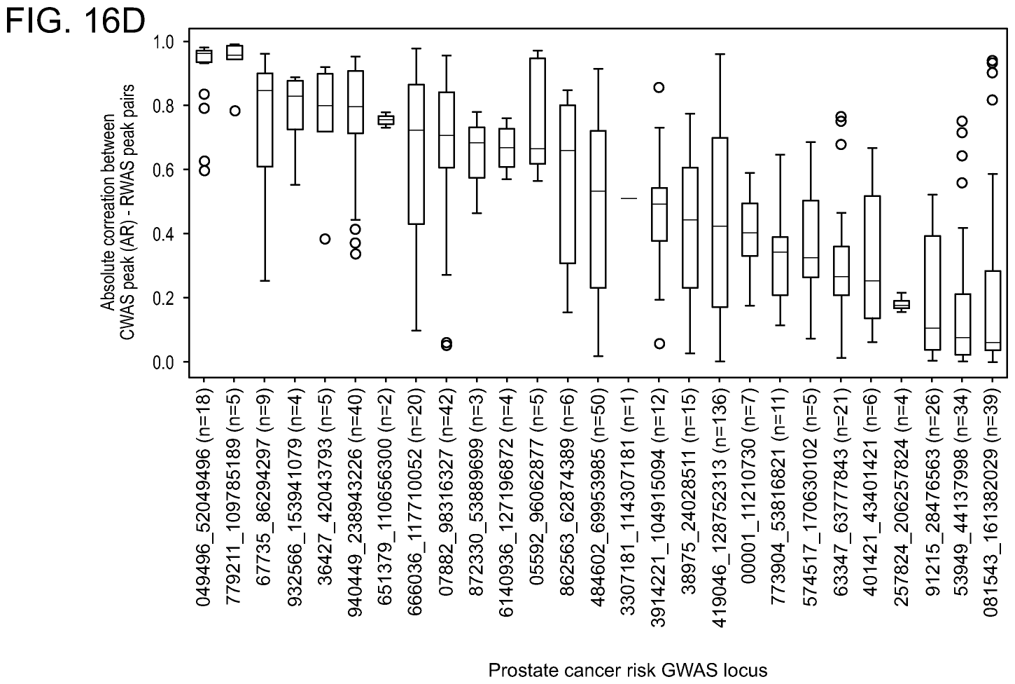

RWAS continued to outperform TWAS even when restricted to equivalent model types (FIG. 14D). RWAS-TWAS correlations were similar for TWAS models from normal tissues, consistent with the observations that cancer as-aQTLs influence expression in normal/healthy tissues (FIG. 14F). The RWAS models were also integrated with “Cistrome-Wide” Associations (CWAS) models that were generated from transcription factor and histone modification ChIP-seq data in prostate cancers. Prostate cancer CWAS associations largely converged on a subset of prostate cancer RWAS loci and yielded correlated models at most of these loci (FIG. 16A-FIG. 16D). In summary, RWAS can identify putative causal mechanisms for the majority of cancer GWAS loci and substantially more than conventional TWAS. Likewise, for the majority of TWAS loci, RWAS can connect TWAS genes to associated regulatory elements.

### Example 11: Allelic Imbalance and RWAS Identify Candidate Causal Risk Variants

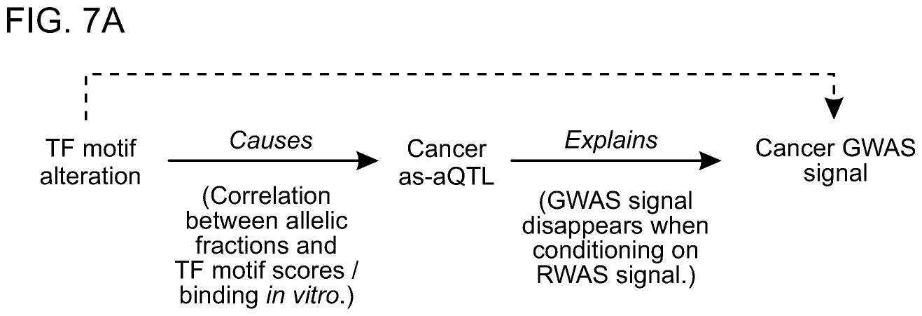

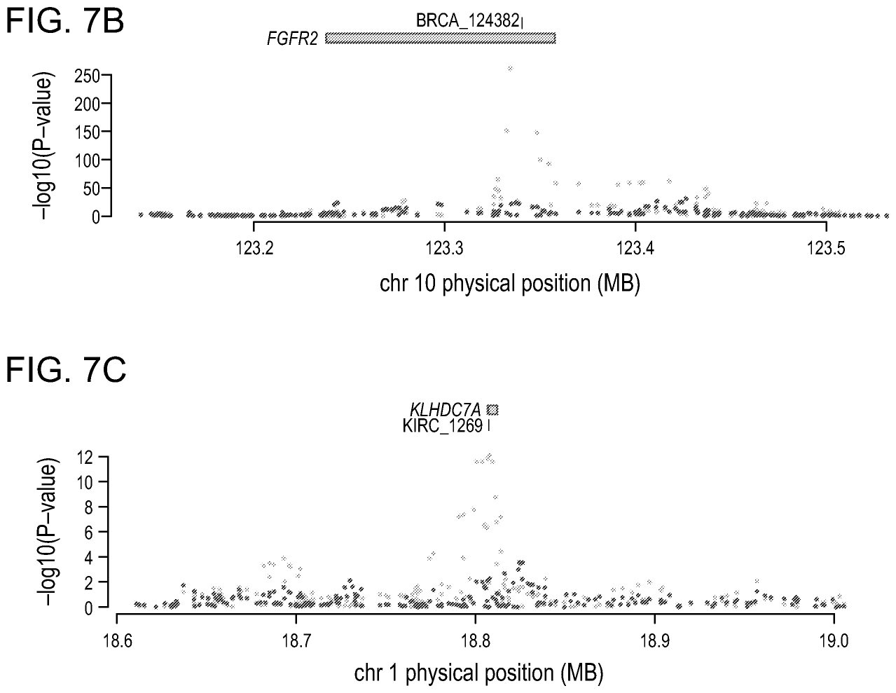

Shown herein is that 1) as-aQTLs are enriched for eQTLs and cancer risk heritability, that 2) allelic imbalance is typically caused by allele-specific alteration of TF motifs, and that 3) RWAS analysis can connect accessible peaks to cancer risk. Finally, set forth herein is a model that combines these analyses to identify candidate causal cancer risk variants and TFs which mediate their effects (FIG. 7A). Using this model, candidate causal variants were identified in upstream TFs at 148 loci across the seven cancer types (Table 8A-Table 8G). The biomarkers in Table 8A-Table 8G represent a subset of variants listed in Table 2A-Table 2W. In addition to being imbalanced variants, the biomarkers in Table 8A-Table 8G are also at peaks associated with cancer risk as well as disrupt one or more TF motifs. Table 8A-Table 8G include information relating to each biomarker, as identified by an rsID number (column labeled ‘RSID’). Table 8A-Table 8G also list, for each biomarker, the chromosome number (column labeled ‘CHR’) and position of the biomarker (column labeled ‘VAR.P1’), the reference nucleotide (column labeled ‘REF’), the altered nucleotide (column labeled ‘ALT’), the TF motif in which the biomarker is found (column labeled ‘MOTIF’), the nearest COSMIC gene (column labeled ‘NEAREST.COSMIC.GENE’) and the genomic distance to that gene (column labeled ‘NEAREST.COSMIC.GENE.DISTANCE’), as well as the nearest gene (which may not be a COSMIC gene) (column labeled ‘NEAREST. GENE’), and the distance to that gene (column labeled ‘NEAREST.GENE.DISTANCE’).

### Table 8A-Table 8G: Integration of as-aQTLs Discovery, Motif Analysis and RWAS Results from 7 Cancer Types

Table 8A-Table 8G are located within the Appendix to the Specification at pages 242 to 391.

Breast cancer risk variants near genes that are recurrently mutated in cancer (from the COSMIC database (Baca, S. C. et al., bioRxiv 2021.05.10.443466 (2021))) are highlighted in this model and are thus likely to be involved in oncogenic processes, as illustrated in Table 9, which shows variants near COSMIC genes with particularly strong evidence for being causal for breast cancer risk. Some peaks have multiple variants that are candidates for being causal. Of note, GWAS signals were often explained best by different RWAS model types. This highlights the benefit of training models on both total and allelic accessibility as well as using the single best and all SNPs in the locus.

For example, the analysis identified rs2981578 as a putative cancer risk causal variant. It is located inside an intron of FGFR2, an oncogene that is somatically altered in multiple cancers. It is also significantly associated with breast cancer risk in GWAS (p=1.3×10⁻²⁴⁵) and was one of the earliest GWAS loci identified in African Americans (Forbes, S. A. et al., Nucleic Acids Res. 39, D945-50 (2011)). rs2981578, which is located inside the BRCA_124382 as-aQTL, was discovered to be significantly imbalanced in breast cancer samples (p =5.8×104).

A lower allelic fraction was observed at its ALT allele (allelic fraction (AF)=0.402) which disrupted the binding motif of the transcription factor RUNX (HOMER motif score decreased from 7.59 to 0.00), which was previously linked to breast cancer (Pasaniuc, B. et al., PLOS Genet. 7, e1001371 (2011)). RWAS likewise identified a significant association with breast cancer risk for this peak (p_{RWAS}<10⁻²⁰⁰), and conditioning on the best RWAS model (top1.combined), explained 97.1% of the GWAS signals in the locus (FIG. 7B). Therefore, rs2981578 is a candidate causal risk variant that acts through differential binding of the RUNX transcription factor. Indeed, the hypothesis was experimentally validated in a previous functional analysis of this region, finding that allele-specific up-regulation of FGFR2 is mediated by differential binding of RUNX at rs2981578, thereby increasing breast cancer risk (Chimge, N.-O. & Frenkel, B. Oncogene 32, 2121-2130 (2013)). More broadly, a recent breast cancer GWAS study provides functional evidence for four risk loci (Michailidou, K. et al., Nature 551, 92-94 (2017)), of which three contained an RWAS association in the analysis (near KLHDC7A, CUX1, and PIDD1), with the validated variant rs2992756, located 84 bp upstream of KLHDC7A, supported by significant imbalance and motif disruption in the data (Table 8A, FIG. 7C). In full, the collection of putative susceptibility elements provides a rich, pan-cancer resource for functional follow-up.

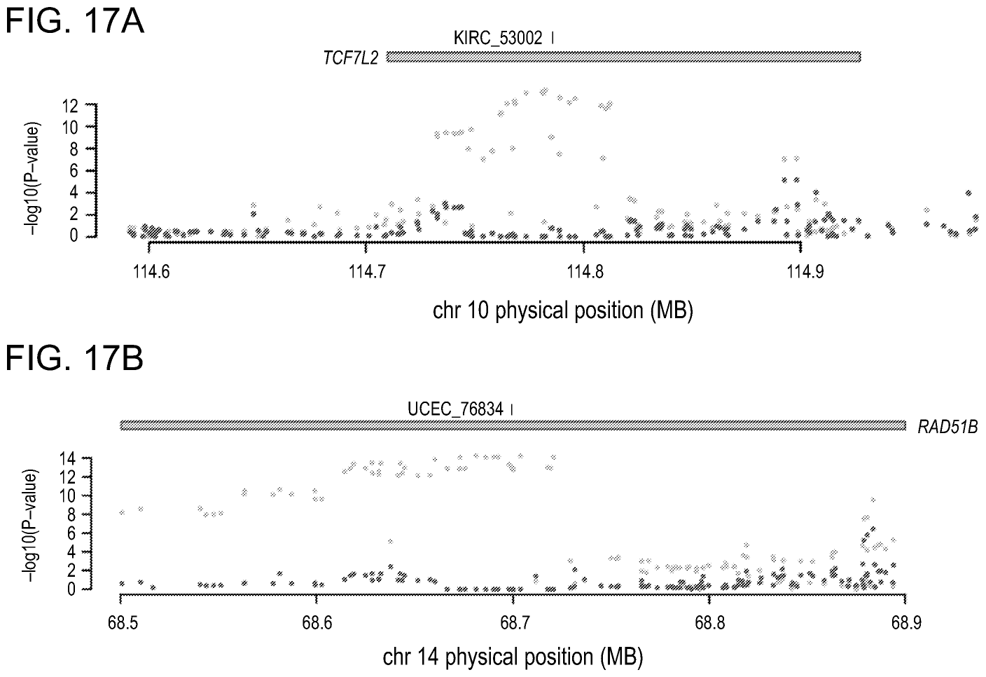

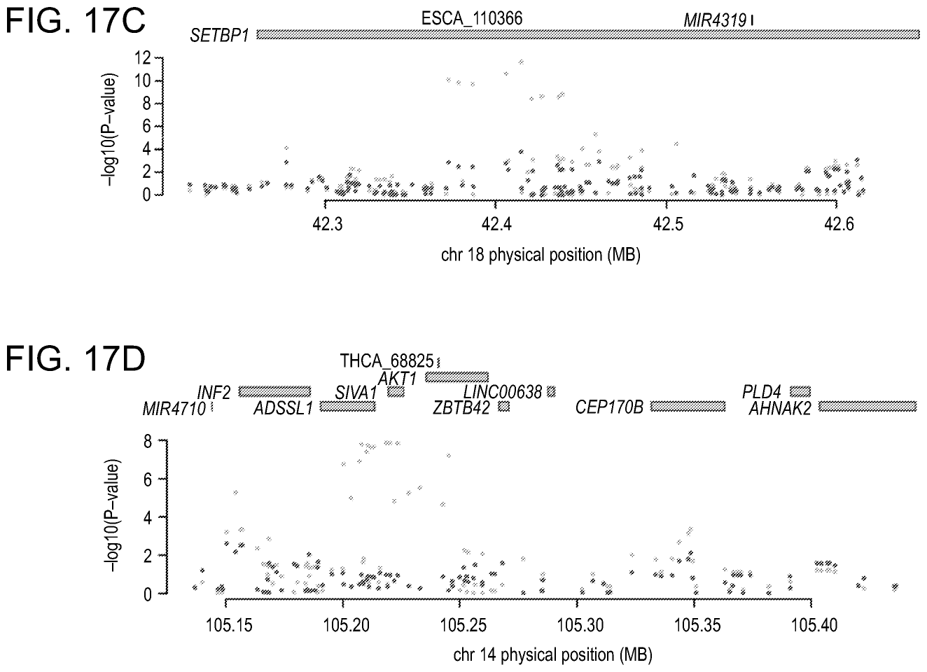

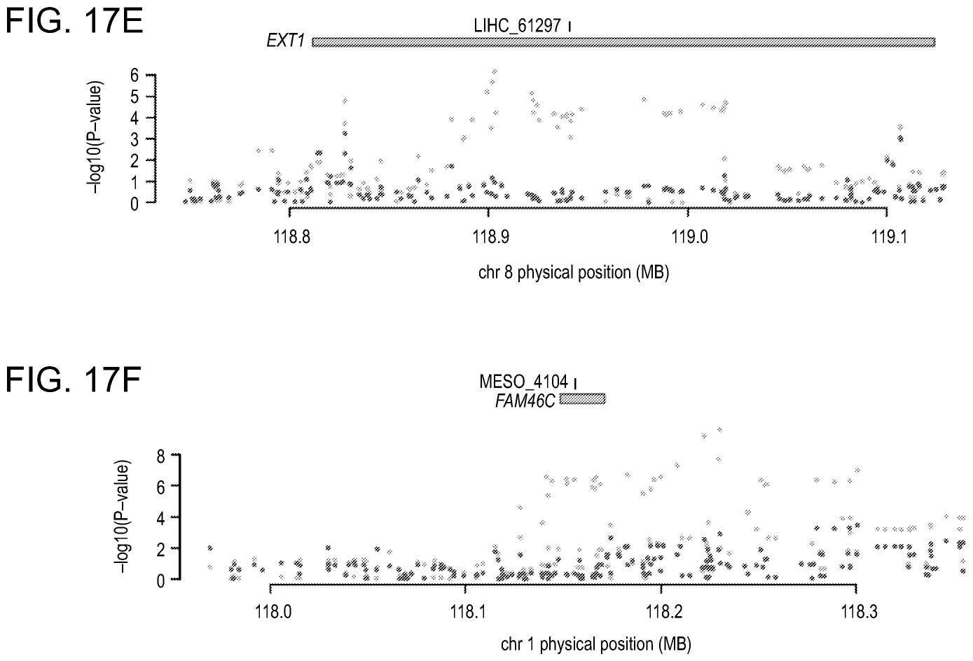

TABLE 9
Candidate causal breast cancer risk variants identified by integrating stratAS, motif
analysis and RWAS.

| | | | | RWAS | RWAS signal | Near |
|---|---|---|---|---|---|---|
| | Significant | Motif | GWAS | significance | explains GWAS | COSMIC |
| | as-aQTL | disruption | significance | (best model; p- | signal (%; | gene |
| rsID* | (name; p-value) | (TF) | (p-value) | value) | Figure) | (name) |
| rs2981578, | BRCA_124382; | Runt and | 1.31 × 10⁻²⁴⁵, | top1.combined; | 97.1%; | FGFR2 |
| rs11599804 | 0.0005804 | ETS | 2.21 × 10⁻³⁰⁷ | <1 × 10⁻²⁰⁰ | FIG. 17B | |
| | | families | | | | |
| rs10787473, | KIRC_53002; | AP2 family | 1.21 × 10⁻¹³, | top1.allelic; | 100%; | TCF7L2 |
| rs12258200 | 1.47 × 10⁻⁵ | | 1.90 × 10⁻¹³ | 2.37 × 10⁻¹³ | FIG. 17A | |
| rs 1316014, | UCEC_76834; | bZIP family | 2.41 × 10⁻¹⁴, | lasso.total; | 100%; | RAD51B |
| rs1314913 | 3.18 × 10⁻¹⁰, | | 1.98 × 10⁻¹³ | 1.84 × 10⁻¹⁴ | FIG. 17B | |
| | 7.65 × 10⁻¹¹ | | | | | |
| rs62090606 | ESCA_110366; | ETS family | 6.84 × 10⁻⁹ | lasso.combined; | 72.8%; | SETBP1 |
| | 8.12 × 10⁻⁷ | | | 1.45 × 10⁻⁹ | FIG. 17C | |
| rs249473, | THCA_68825; | Zf and E2F | 4.08 × 10⁻⁵, | lasso.combined, | 91.9%; | AKT1 |
| rs2494734 | 6.42 × 10⁻⁵, | families | 1.68 × 10⁻⁷ | 1.03 × 10⁻⁸ | FIG. 17D | |
| | 1.50 × 10⁻⁶ | | | | | |
| rs 1462985 | LIHC_61297; | E2F family | 0.0006261 | lasso.allelic; | 89.6%; | EXT1 |
| | 4.05 × 10⁻⁴ | | | 1.07 × 10⁻⁶ | FIG. 17E | |
| rs3767812 | MESO_4104; | bZIP family | 6.66 × 10⁻⁷ | lasso.allelic; | 73.9%; | FAM46C |
| | 7.55 × 10⁻⁸ | | | 8.27 × 10⁻¹⁰ | FIG. 17F | |

*rsID (Reference SNP cluster ID) is a unique number used to identify a specific SNP and is the naming convention used for most SNPs. Alternative SNP identification systems includes genome build and chromatin position identifiers.

The working examples disclosed herein demonstrate that allelic imbalance analysis and imputation of chromatin accessibility in cancer samples can help identify causal germline cancer risk variants and explain their mechanisms. The working examples show that cancer as-aQTLs are strongly enriched for cancer risk heritability, often exhibiting almost an order of magnitude stronger enrichment than the next best annotation (including eQTLs). These as-aQTLs are likely involved in the regulation of gene expression as they are enriched for eQTLs, are often located in promoters, and implicate genetic variants that alter binding motifs of TFs, many of which have been linked to dysregulation in cancer. By integrating with data from TF binding and expression measurement assays, as-aQTLs were shown to be characterized by differential TF binding that affects gene expression. Finally, a new association study (RWAS) that connects as-aQTLs with cancer risk was developed and applied, providing putative mechanisms for the majority of known cancer risk loci, and identifies novel loci. The results constitute a pan-cancer database of putative risk mechanisms to enable the visualization of connections between regulatory elements and genes. Along multiple axes, these results establish cancer as-aQTLs as the most relevant known annotation for understanding cancer risk heritability and mechanisms. These data support previously hypothesized germline-somatic interactions (Houlahan, K. E. et al., Nat. Med. 25, 1615-1626 (2019)) whereby germline cancer risk variants continue to play a role in epigenetic dysregulation in cancers.

Additionally, the power to discover cancer-specific as-aQTLs may be further increased by incorporating cancer sample purity as a parameter or interaction term (Geeleher, P. et al., Genome Biol. 19, 130 (2018); Kalita, C. A. & Gusev, A. bioRxiv 2021.11.11.468278 (2021)), which would discount the contribution of impure cancer samples. Second, the benchmarking indicated that very deep somatic CNVs were associated with a very pronounced allelic imbalance in chromatin accessibility in a small fraction of samples. These very deep CNV regions were conservatively excluded from the primary analysis, which lowered the number of detected as-aQTLs, but this approach may led to false negatives. The analysis was limited to common germline SNPs and did not investigate possible as-aQTLs at somatic point mutations. One well-known example of a non-coding mutation that drives cancerogenesis is mutations at the telomerase gene (TERT) promoter that create a new binding motif for ETS family transcription factors and drive telomerase gene expression (Corces, M. R. et al., Science 362, (2018); Horn, S. et al., Science 339, 959-961 (2013)). This mechanism is very similar to the germline SNP-driven as-aQTLs discovered in this study, many of which were caused by the alteration of ETS motifs, suggesting that somatic as-aQTLs may also be used to study non-coding driver mutations in cancer.

In the search for GWAS mechanisms, it has become common practice to integrate associations with eQTL/TWAS (Gamazon, E. R. et al., Nat. Genet. 50, 956-967 (2018)) or epigenetic features (Boix, C. A., et al., Nature 590, 300-307 (2021)). However, the majority of GWAS loci do not contain an eQTL colocalization or a TWAS association (GTEx Consortium. Science 369, 1318-1330 (2020); Mancuso, N. et al., Am. J. Hum. Genet. 100, 473-487 (2017)), even when the cell type in which gene expression was measured matches the expected disease mechanism (Chun, S. et al., Nat. Genet. 49, 600-605 (2017)); and the total fraction of disease heritability estimated to be mediated by all measurable eQTLs is similarly low (Yao, D. W. et al., Nat. Genet. 52, 626-633 (2020)). This question of the “missing mechanism” has motivated looking at alternative molecular features (Umans, B. D. et al., Trends Genet. 37, 109-124(2021)): highly cell-type-specific expression (Bonder, M. J. et al., Nat. Genet. 53, 313-321 (2021)), dynamic or context-specific eQTLs (Strober, B. J. et al., 364, 1287-1290 (2019)), trans-QTLs (Võsa, U. et al., bioRxiv 447367 (2018)), and chromatin QTLs (Waszak, S. M. et al., Cell 162, 1039-1050 (2015); Grubert, F. et al., Cell 162, 1051-1065 (2015); Gate, R. E. et al., Nat. Genet. 50, 1140-1150 (2018)). Alternatively, some putative mechanisms have been implicated by intersecting risk variants with epigenomic features agnostic of a genetic effect on the feature (Boix, C. A., James, B. T., Park, Y. P., Meuleman, W. & Kellis, M. Nature 590, 300-307 (2021); Fulco, C. P. et al., Nat. Genet. 51, 1664-1669 (2019); Nasser, J. et al., Nature 593, 238-243 (2021); Weissbrod, O. et al., Nat. Genet. 52, 1355-1363 (2020)); under the assumption that a risk variant in a functional feature is likely to be acting through that feature. The working examples disclosed herein bridge these two frameworks by identifying variants that are associated with disease and with an effect on chromatin accessibility in malignant cells. Across multiple cancers, as-aQTLs/RWASs demonstrated increased sensitivity over steady-state eQTL models to identify putative mechanisms, as well as increased specificity over generic accessible peaks (as quantified by heritability enrichment analyses). Taken together, these findings suggest that population-scale profiling of epigenomic features from cancers can help close the “missing mechanism gap” in cancer GWAS.

All patent publications and non-patent publications are indicative of the level of skill of those skilled in the art to which this disclosure pertains. All these publications (including any specific portions thereof that are referenced) are herein incorporated by reference to the same extent as if each individual publication were specifically and individually indicated as being incorporated by reference.

Although the disclosure herein has been described with reference to particular embodiments, it is to be understood that these embodiments are merely illustrative of the principles and applications of the present disclosure. It is therefore to be understood that numerous modifications may be made to the illustrative embodiments and that other arrangements may be devised without departing from the spirit and scope of the present disclosure as defined by the appended claims.

Lengthy table referenced here
US20250372199A1-20251204-T00001
Please refer to the end of the specification for access instructions.

LENGTHY TABLES
The patent application contains a lengthy table section. A copy of the table is available in electronic form from the USPTO web site (https://seqdata.uspto.gov/docdetail?docId=US20250372199A1). An electronic copy of the table will also be available from the USPTO upon request and payment of the fee set forth in 37 CFR 1.19(b)(3).

1.-40. (canceled)

41. A method, comprising:
receiving sequencing data for a plurality of accessibility features;
generating a predictive model using the sequencing data to predict germline genetic variants associated with one or more of the plurality of accessibility features that result in allelic imbalance, the predictive model trained using transcription-factor (TF) binding accessibility;
inputting sample data into the predictive model; and
outputting the predicted germline genetic variants that result in the allelic imbalance.

42. The method of claim 41, wherein the germline genetic variants comprise allele specific accessibility quantitative trait loci (as-aQTLs).

43. The method of claim 42, further comprising predicting one or more cancer-type specific as-aQTLs for one or more different cancer types.

44. The method of claim 43, wherein the one or more different cancer types comprise one or more of breast cancer, colorectal cancer, prostate cancer, lung cancer, kidney cancer, glioma, and melanoma.

45. The method of claim 41, further comprising updating the predictive model based on the sample data.

46. The method of claim 41, wherein the predictive model comprises one of a plurality of predictive model types.

47. The method of claim 41, further comprising identifying accessibility quantitative trait loci (as-aQTLs) that are associated with cancer riskfor each accessibility feature based on the trained predictive model.

48. The method of claim 47 wherein generating the predictive model comprises weighting each accessibility feature based on the identified as-aQTLs.

49. The method of claim 47, further comprising outputting the predicted germline genetic variants that result in the allelic imbalance based the identified as-aQTLs.

50. The method of claim 41, wherein training a predictive model comprises training a plurality of different predictive models.

51. The method of claim 49, further comprising selecting one of the plurality of models based on determining its correlation with a most predictive model for each feature.

52. A method of generating a predictive model for determining genomic regions that increase cancer risk heritability, comprising:
receiving sequencing data for a plurality of accessibility features;
training a predictive model using transcription-factor (TF) binding accessibility to determine genetic predictors for each accessibility feature of the plurality of accessibility features; and
identifying accessibility quantitative trait loci (as-aQTLs) that are associated with cancer risk for each accessibility feature based on the trained predictive model.

53. The method of claim 52, wherein training a predictive model comprises training a plurality of different predictive models.

54. The method of claim 53, further comprising selecting one of the plurality of models based on determining its correlation with a most predictive model for each accessibility feature.

55. The method of claim 52, further comprises weighting each feature based on the identified as-aQTLs.

56. The method of claim 52, wherein the germline genetic variants comprise allele specific accessibility quantitative trait loci (as-aQTLs).

57. The method of claim 56, further comprising predicting one or more cancer-type specific as-aQTLs for one or more different cancer types.

58. The method of claim 52, wherein training the predictive model comprises training the predictive model to predict germline genetic variants associated with one or more of the plurality of accessibility features.

59. A system, comprising:
one or more storage devices configured to store sequencing data for a plurality of accessibility features, and a processor coupled to the one or more storage devices, the processor configured to:
receive the sequencing data for a plurality of accessibility features;
generate a predictive model using the sequencing data to predict germline genetic variants associated with one or more of the plurality of accessibility features that result in allelic imbalance, the predictive model trained using transcription-factor (TF) binding accessibility;
input sample data into the predictive model; and
output the predicted germline genetic variants that result in the allelic imbalance.

60. The system of claim 59, wherein the germline genetic variants comprise allele specific accessibility quantitative trait loci (as-aQTLs).
